(19) United States
(12) Patent Application Publication
(10) Pub. No.: US 2026/0021084 A1
(43) Pub. Date: Jan. 22, 2026

(54) METHOD OF TREATING ORGAN DISEASES OR DISORDERS WITH ASK1 INHIBITORS

(71) Applicant: Seal Rock Therapeutics, Inc., Seattle, WA (US)

(72) Inventors: Artur PLONOWSKI, Cupertino, CA (US); Kathleen Ann ELIAS, San Francisco, CA (US); Neil Dwayne MCDONNELL, Seattle, WA (US); Samuel David BROWN, San Carlos, CA (US); Terence Graham PORTER, Dunedin, FL (US)

(21) Appl. No.: 18/996,525

(22) PCT Filed: Jul. 19, 2023
(86) PCT No.: PCT/US2023/070517

§ 371 Date: Jan. 17, 2025

Related U.S. Application Data
(60) Provisional application No. 63/368,961, filed on Jul. 20, 2022.

(51) Int. Cl. A61K 31/4439; A61K 9/00; A61K 31/197; A61K 31/4545; A61K 31/496; A61K 31/497; A61K 31/501; A61K 31/506; A61K 31/5377; A61K 31/5386; A61K 31/541; A61K 45/06; A61P 1/16

(52) U.S. Cl. CPC A61K 31/4439; A61K 9/0019; A61K 31/197; A61K 31/4545; A61K 31/496; A61K 31/497; A61K 31/501; A61K 31/506; A61K 31/5377; A61K 31/5386; A61K 31/541; A61K 45/06; A61P 1/16

(57) ABSTRACT

The present invention relates to the use of Apoptosis Signal-Regulating Kinase-1 (ASK-1) inhibitor compounds for the treatment of organ diseases and disorders, wherein the organ disease or disorder is selected from a liver, heart disease, kidney, pancreas, spleen, or skin disease or disorder.

### CROSS-REFERENCE

This application claims benefit of U.S. Provisional Application No. 63/368,961, filed on Jul. 20, 2022, which is herein incorporated by reference in its entirety.

### BACKGROUND OF THE INVENTION

A variety of disease processes can affect organs such as the liver, kidney, heart, pancreas, spleen, and skin, including liver injury, acute kidney injury, and heart diseases.

### SUMMARY OF THE INVENTION

There remains a need for novel new compounds to treat these organ diseases. ASK1 inhibitors may be useful for treating these organ diseases. Orally dosed ASK1 inhibitors described herein show preferential distribution to liver and kidney, and achieve organ concentration several fold above the plasma concentration. Injectable administration of ASK1 inhibitors described herein also show preferential distribution to liver and kidney and thus are suitable for treatment of patients in whom the injectable route of administration is preferred over the oral route, including patients who are unconscious, have swallowing difficulties or require a rapid onset of pharmacological activity. As such, in some embodiments ASK1 inhibitors described herein are useful for treating organ diseases.

In one aspect, described herein is a method for treating an organ disease or disorder in a subject in need thereof comprising administering to the subject a therapeutically effective amount of compounds, or pharmaceutically acceptable salts or solvates thereof that inhibit ASK1.

In some embodiments described herein is a method for treating an organ disease or disorder in a subject in need thereof comprising administering to the subject a therapeutically effective amount of a compound of Formula IIa, or a pharmaceutically acceptable salt or solvate thereof:

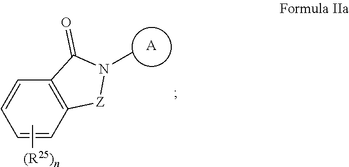

wherein

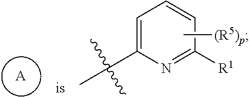

R¹ is

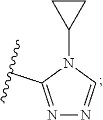 [SMILES: *c1nncn1C1CC1]

Z is C(R⁹)₂;
R⁵ is selected from a group consisting of halogen and C₁₋₆alkyl;
each R²⁵ is independently selected from a group consisting of halogen, —OR⁶, —N(R⁶)₂, —C(═O)OR⁶, —C(═O)N(R⁶)₂, —NR⁶C(═O)N(R⁶)₂, and C₁₋₉heteroaryl selected from pyridinyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl; wherein the C₁₋₉heteroaryl is optionally substituted with one, two, or three substituents selected from the group consisting of halo, C₁₋₆alkyl, C₁₋₆haloalkyl, and C₃₋₈cycloalkyl;
each R⁶ is independently selected from the group consisting of hydrogen, C₁-C₆alkyl, —C₁-C₆alkyl-O—C₁-C₆alkyl, —C₁-C₆alkyl-pyrazole, and C₃-C₈cycloalkyl; or two R⁶ on the same heteroatom are taken together with that heteroatom to which they are attached to form a C₂₋₉heterocycle or a C₂₋₉heteroaryl wherein the heteroaryl is_selected from imidazolyl, pyrazolyl, and pyrrolyl, wherein the C₂₋₉heterocycle or C₂₋₉heteroaryl are optionally substituted with one, two, or three substituents selected from the group consisting of halo, —OR⁸, —SR⁸, —N(R⁸)₂, —C₁₋₆alkyl, —C(═O)R¹⁴, —C(═O)OR¹³, and —N(R¹³)C(═O)R¹⁴;
each R⁸ is independently selected from the group consisting of hydrogen and C₁-C₆alkyl;
each R⁹ is hydrogen;
each R¹³ is independently selected from the group consisting of hydrogen and C₁-C₆alkyl;
each R¹⁴ is independently selected from the group consisting of C₁-C₆alkyl;
n is 0, 1, or 2; and
p is 0 or 1.

In some embodiments, n is 1.

In some embodiments, R²⁵ is —C(═O)N(R⁶)₂ and each R⁶ is independently selected from the group consisting of hydrogen, C₁-C₆alkyl, —C₁-C₆alkyl-O—C₁-C₆alkyl, —C₁-C₆alkyl-pyrazole, and C₃-C₈cycloalkyl.

In some embodiments, R²⁵ is —C(═O)N(R⁶)₂ and two R⁶ on the same heteroatom are taken together with that heteroatom to which they are attached to form a C₂₋₉heterocycle or a C₂₋₉ heteroaryl wherein the heteroaryl is selected from imidazolyl, pyrazolyl, and pyrrolyl, wherein the C₂₋₉heterocycle or C₂₋₉heteroaryl are optionally substituted with one substituent selected from the group consisting of —OR⁸, —SR⁸, —N(R⁸)₂, —C₁₋₆alkyl, —C(═O)R¹⁴, —C(═O)OR¹³, and —N(R¹³)C(═O)R¹⁴.

In some embodiments, R²⁵ is

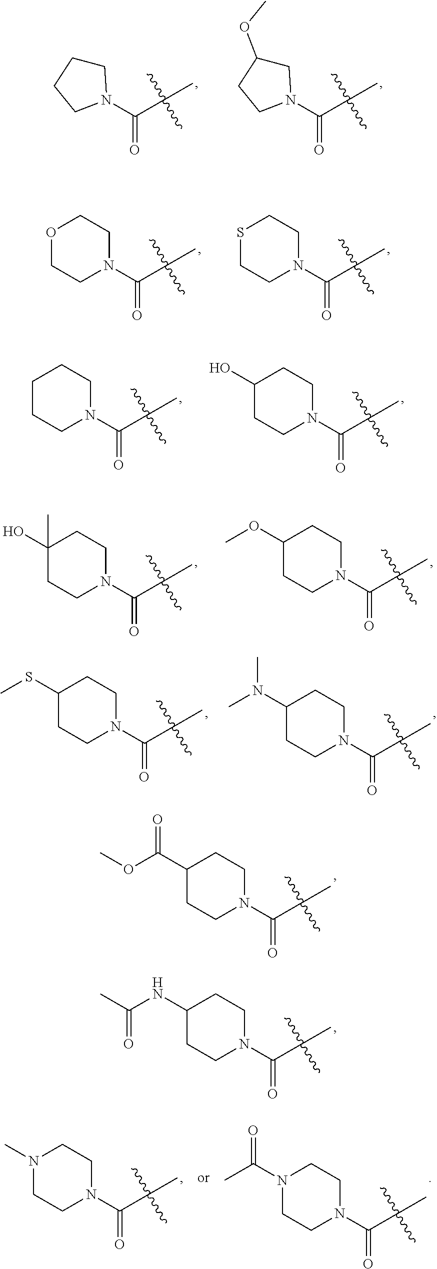

In some embodiments, R²⁵ is

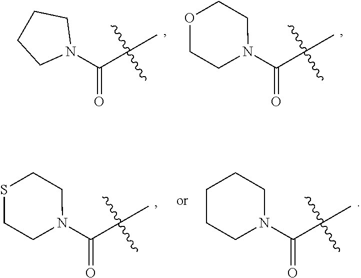

In some embodiments, presented herein is a method for treating an organ disease or disorder in a subject in need thereof comprising administering to the subject a therapeutically effective amount of a compound of Formula I, or a pharmaceutically acceptable salt or solvate thereof:

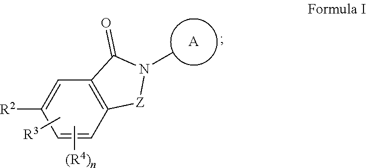

wherein

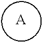

is

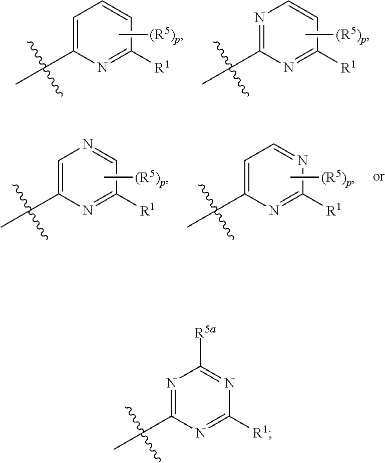

Z is O, S, C(═O), N(R⁸), or C(R⁹)₂;
R¹ and R³ are each independently selected from a group consisting of hydrogen, halogen, —CN, —OH, —OR⁶, —SR⁶, —S(═O)R⁷, —NO₂, —N(R⁶)₂, —S(═O)₂R⁷, —NHS(═O)₂R⁷, —S(═O)₂N(R⁶)₂, —C(═O)R⁷, —C(═O)OR⁶, —OC(═O)R⁷, —C(═O)N(R⁶)₂, —OC(═O)N(R⁶)₂, —NR⁶C(═O)N(R⁶)₂, —NR⁶C(═O)R⁷, —NR⁶C(═O)OR⁶, C₁₋₆alkyl, C₂₋₆alkenyl, C₂₋₆alkynyl, C₃₋₈cycloalkyl, C₂₋₉heterocycle, C₆₋₁₀aryl, C₁₋₉heteroaryl, and a fused C₅₋₉heteroaryl-cycloalkyl; wherein C₁₋₆alkyl, C₂₋₆alkenyl, C₂₋₆alkynyl, C₃₋₈cycloalkyl, C₂₋₉heterocycle, C₆₋₁₀aryl, C₁₋₉heteroaryl, and fused C₅₋₉heteroaryl-cycloalkyl are optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³; R² is selected from a group consisting of hydrogen, halogen, —CN, —OH, —SR⁶, —S(═O)R⁷, —NO₂, —N(R⁶)₂, —S(═O)₂R⁷, —NHS(═O)₂R⁷, —S(═O)₂N(R⁶)₂, —C(═O)R⁷, —C(═O)OR⁶, —OC(═O)R⁷, —C(═O)N(R⁶)₂, —OC(═O)N(R⁶)₂, —NR⁶C(═O)N(R⁶)₂, —NR⁶C(═O)R⁷, —NR⁶C(═O)OR⁶, C₁₋₆alkyl, C₂₋₆alkenyl, C₂₋₆alkynyl, C₃₋₈cycloalkyl, C₂₋₉heterocycle, C₆₋₁₀aryl, C₁₋₉heteroaryl, and a fused C₅₋₉heteroaryl-cycloalkyl; wherein C₁₋₆alkyl, C₂₋₆alkenyl, C₂₋₆alkynyl, C₃₋₈cycloalkyl, C₂₋₉heterocycle, C₆₋₁₀aryl, C₁₋₉heteroaryl, and fused C₅₋₉heteroaryl-cycloalkyl are optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³; wherein R² and R³ are not both hydrogen;
each R⁴ and each R⁵ are each independently selected from a group consisting of halogen, —CN, and C₁₋₆alkyl;
R^{5a} is selected from the group consisting of hydrogen and C₁-C₆alkyl;
each R⁶ is independently selected from the group consisting of hydrogen, C₁-C₆alkyl, —C₁-C₆alkyl-O—C₁-C₆alkyl, —C₁-C₆alkyl-C₂₋₉heterocycle, —C₁-C₆alkyl-C₂₋₉heteroaryl, C₃-C₈cycloalkyl, and C₂₋₉heterocycle; or two R⁶ on the same heteroatom are taken together with that heteroatom to which they are attached to form a C₂₋₉heterocycle or a C₂₋₉heteroaryl;
each R⁷ is independently selected from the group consisting of C₁-C₆alkyl and C₃-C₈cycloalkyl;
R⁸ is selected from the group consisting of hydrogen and C₁-C₆alkyl;
each R⁹ is independently selected from the group consisting of hydrogen, halogen, and C₁-C₆alkyl;
each R¹³ is independently selected from the group consisting of hydrogen, C₁-C₆alkyl, and C₃-C₈cycloalkyl; or two R¹³ on the same heteroatom are taken together with that heteroatom to which they are attached to form a C₂₋₉heterocycle;
each R¹⁴ is independently selected from the group consisting of C₁-C₆alkyl and C₃-C₈cycloalkyl;
n is 0, 1, or 2;
p is 0, 1, 2, or 3; and
q is 0, 1, or 2.

In some embodiments, R² is selected from a group consisting of C₃₋₈cycloalkyl, C₂₋₉heterocycle, C₆₋₁₀aryl, C₁₋₉heteroaryl, and a fused C₅₋₉heteroaryl-cycloalkyl; wherein C₃₋₈cycloalkyl, C₂₋₉heterocycle, C₆₋₁₀aryl, C₁₋₉heteroaryl, and fused C₅₋₉heteroaryl-cycloalkyl are optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R² is selected from a group consisting of C₂₋₉heterocycle and C₁₋₉heteroaryl; wherein C₂₋₉heterocycle and C₁₋₉heteroaryl are optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R² is selected from a group consisting of C₂₋₉heterocycle and C₁₋₉heteroaryl; wherein C₂₋₉heterocycle and C₁₋₉heteroaryl are optionally substituted with one or two substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R² is selected from a group consisting of pyrazole, imidazole, thiazole, and pyridine; wherein pyrazole, imidazole, thiazole, and pyridine are optionally substituted with one or two substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R² is selected from a group consisting of pyrazole, imidazole, thiazole, and pyridine; wherein pyrazole, imidazole, thiazole, and pyridine are optionally substituted with one or two substituents selected from the group consisting of halo, C₁₋₆alkyl, and C₃₋₈cycloalkyl. In some embodiments, R² is

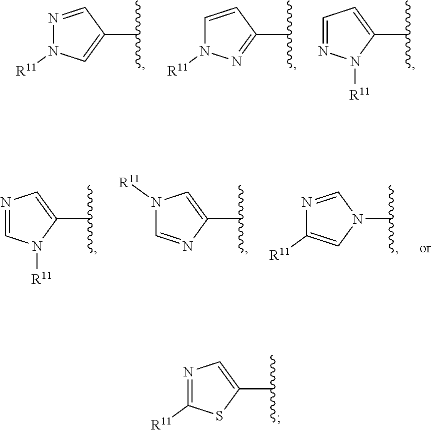

wherein R¹¹ is C₁-C₆alkyl or C₃-C₆cycloalkyl.

In some embodiments, R² is

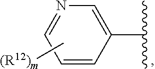

wherein R¹² is halo, C₁-C₆alkyl, or C₃-C₆cycloalkyl; and m is 1 or 2.

In some embodiments, R² is selected from a group consisting of unsubstituted pyrazole, unsubstituted imidazole, unsubstituted thiazole, and unsubstituted pyridine.

In some embodiments, R² is —C(═O)N(R⁶)₂ and each R⁶ is independently selected from the group consisting of hydrogen, C₁-C₆alkyl, —C₁-C₆alkyl-O—C₁-C₆alkyl, —C₁-C₆alkyl-C₂₋₉heterocycle, —C₁-C₆alkyl-C₂₋₉heteroaryl, C₃-C₈cycloalkyl, and C₂₋₉heterocycle.

In some embodiments, R² is

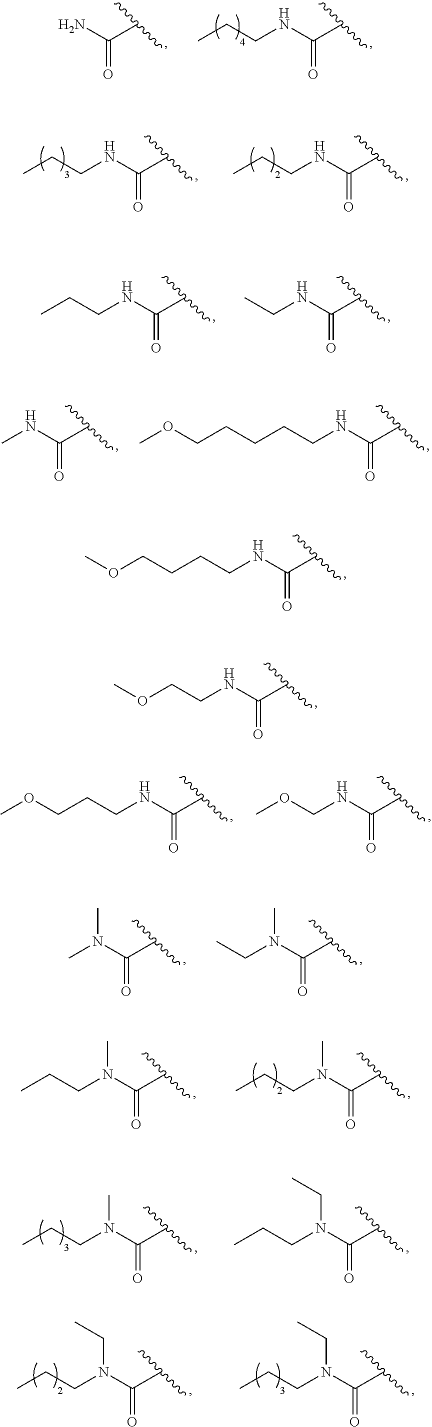

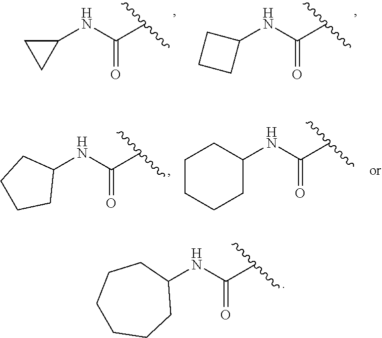

In some embodiments, R² is

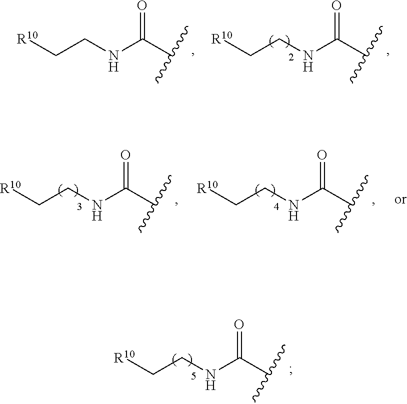

wherein R¹⁰ is a heteroaryl.

In some embodiments, R³ is hydrogen. In some embodiments, R³ is C₁-C₆alkyl.

In some embodiments, R³ is selected from a group consisting of C₃₋₈cycloalkyl, C₂₋₉heterocycle, C₆₋₁₀aryl, C₁₋₉heteroaryl, and a fused C₅₋₉heteroaryl-cycloalkyl; wherein C₃₋₈cycloalkyl, C₂₋₉heterocycle, C₆₋₁₀aryl, C₁₋₉heteroaryl, and fused C₅₋₉heteroaryl-cycloalkyl, wherein C₃₋₈cycloalkyl, C₂₋₉heterocycle, C₆₋₁₀aryl, C₁₋₉heteroaryl, and fused C₅₋₉heteroaryl-cycloalkyl are optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R³ is selected from a group consisting of C₂₋₉heterocycle and C₁₋₉heteroaryl; wherein C₂₋₉heterocycle and C₁₋₉heteroaryl are optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R³ is selected from a group consisting of C₂₋₉heterocycle and C₁₋₉heteroaryl; wherein C₂₋₉heterocycle and C₁₋₉heteroaryl are optionally substituted with one or two substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R³ is selected from a group consisting of pyrazole, imidazole, thiazole, and pyridine; wherein pyrazole, imidazole, thiazole, and pyridine are optionally substituted with one or two substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R³ is selected from a group consisting of pyrazole, imidazole, thiazole, and pyridine; wherein pyrazole, imidazole, thiazole, and pyridine are optionally substituted with one or two substituents selected from the group consisting of halo, C₁₋₆alkyl, and C₃₋₈cycloalkyl.

In some embodiments, R³ is

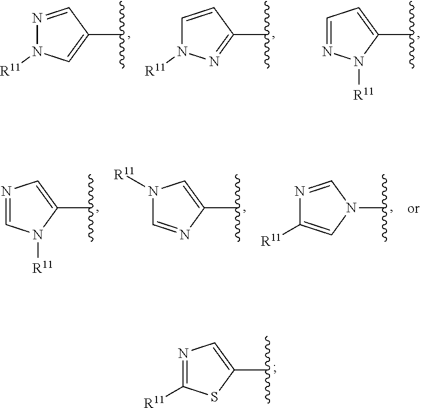

wherein R¹¹ is C₁-C₆alkyl or C₃-C₆cycloalkyl.

In some embodiments, R³ is

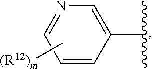

wherein each R¹² is independently halo, C₁-C₆alkyl, or C₃-C₆cycloalkyl; and m is 1 or 2.

In some embodiments, R³ is selected from a group consisting of unsubstituted pyrazole, unsubstituted imidazole, unsubstituted thiazole, and unsubstituted pyridine.

In some embodiments, R³ is —C(═O)N(R⁶)₂ and each R⁶ is independently selected from the group consisting of hydrogen, C₁-C₆alkyl, —C₁-C₆alkyl-O—C₁-C₆alkyl, —C₁-C₆alkyl-C₂₋₉heterocycle, —C₁-C₆alkyl-C₂₋₉heteroaryl, C₃-C₈cycloalkyl, and C₂₋₉heterocycle.

In some embodiments, R³ is

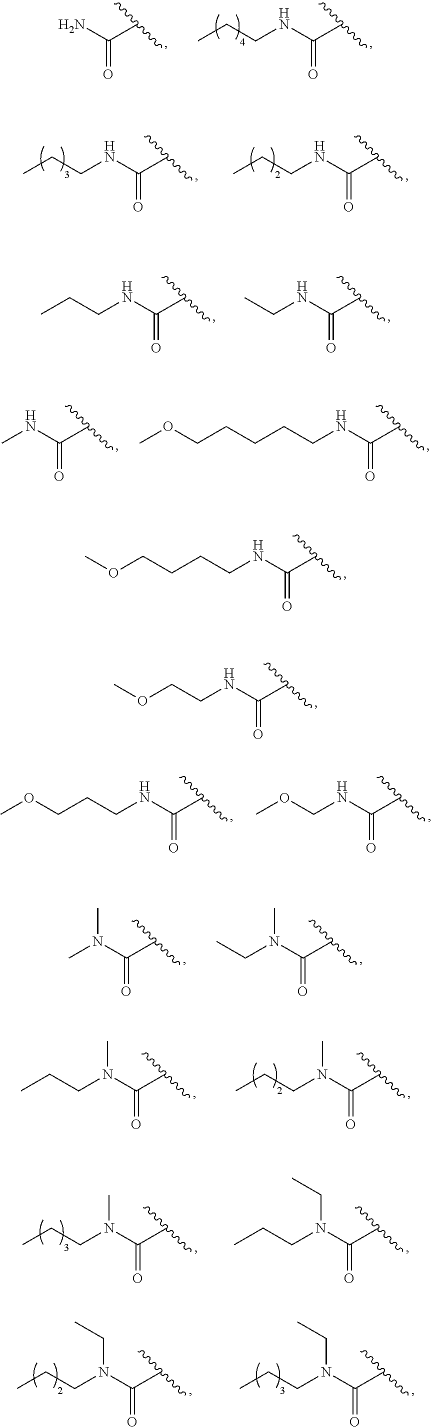

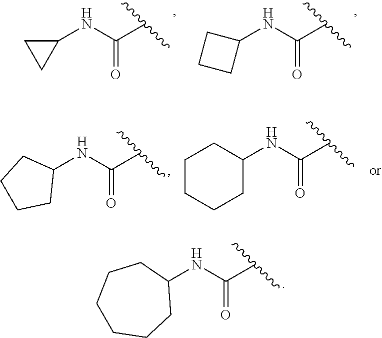

In some embodiments, R³ is

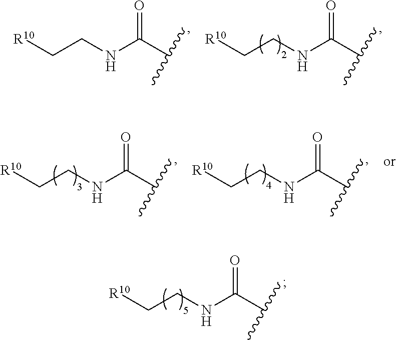

wherein R¹⁰ is a heteroaryl.

In some embodiments, R³ is —OR⁶ and R⁶ is selected from the group consisting of C₁-C₆alkyl, —C₁-C₆alkyl-O—C₁-C₆alkyl, and —C₁-C₆alkyl-C₂₋₉heterocycle. In some embodiments, R² is hydrogen. In some embodiments, R² is C₁-C₆alkyl.

In some embodiments, R¹ is selected from a group consisting of C₃₋₈cycloalkyl, C₂₋₉heterocycle, C₆₋₁₀aryl, C₁₋₉heteroaryl, and a fused C₅₋₉heteroaryl-cycloalkyl; wherein C₁₋₆alkyl, C₂₋₆alkenyl, C₂₋₆alkynyl, C₃₋₈cycloalkyl, C₂₋₉heterocycle, C₆₋₁₀aryl, C₁₋₉heteroaryl, and fused C₅₋₉heteroaryl-cycloalkyl are optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R¹ is selected from a group consisting of a C₁₋₉heteroaryl and a fused C₅₋₉heteroaryl-cycloalkyl; wherein the C₁₋₉heteroaryl and fused C₅₋₉heteroaryl-cycloalkyl are optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R¹ is selected from a group consisting of a C₁₋₉heteroaryl and a fused C₅₋₉heteroaryl-cycloalkyl; wherein the C₁₋₉heteroaryl and fused C₅₋₉heteroaryl-cycloalkyl are optionally substituted with one or two substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R¹ is selected from a group consisting of triazole, imidazole, oxazole, isoxazole, oxadiazole, and tetrazole; wherein triazole, imidazole, oxazole, isoxazole, oxadiazole, and tetrazole are optionally substituted with one or two substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R¹ is selected from a group consisting of triazole, imidazole, oxazole, isoxazole, oxadiazole, and tetrazole; wherein triazole, imidazole, oxazole, isoxazole, oxadiazole, and tetrazole are optionally substituted with one or two substituents selected from the group consisting of halo, C₁₋₆alkyl, and C₃₋₈cycloalkyl.

In some embodiments, R¹ is

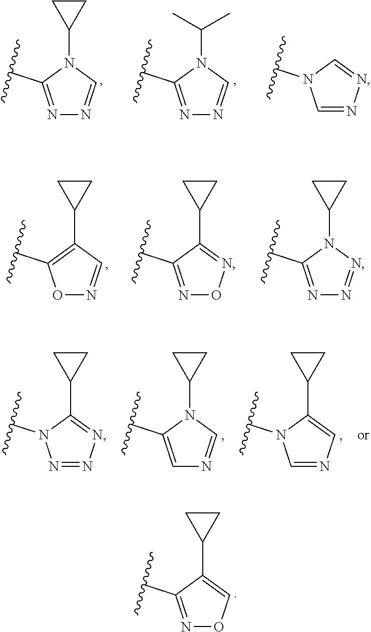

In some embodiments, R¹ is

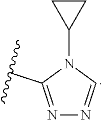 [SMILES: *c1nncn1C1CC1]

In some embodiments, R¹ is

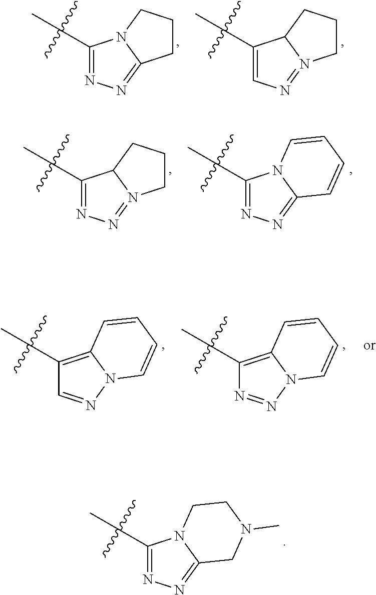

In some embodiments described herein is a method for treating an organ disease or disorder in a subject in need thereof comprising administering to the subject a therapeutically effective amount of a compound of Formula II, or a pharmaceutically acceptable salt or solvate thereof:

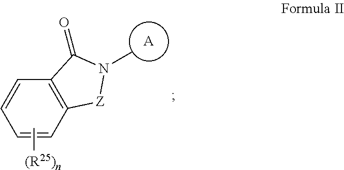

wherein

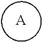

is

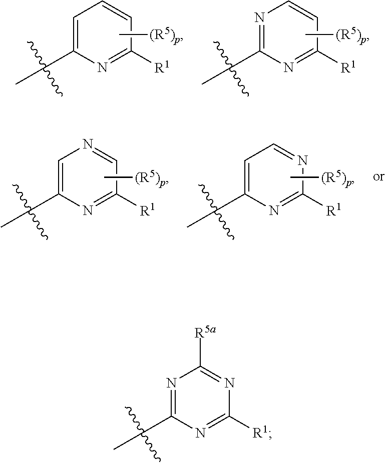

R¹ is

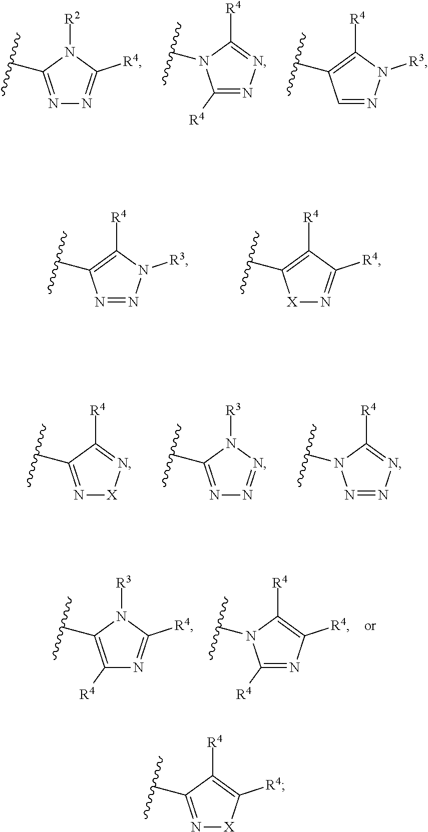

Z is O, S, C(═O), N(R⁸), or C(R⁹)₂;
X is O or S;
R² is C₃₋₆cycloalkyl;
R³ is selected from a group consisting of hydrogen, C₁₋₆alkyl, and C₃₋₆cycloalkyl;
each R⁴ is independently selected from a group consisting of hydrogen, halo, C₁₋₆alkyl, and C₃₋₆cycloalkyl;
   or one R⁴ and another R², R³, or R⁴, together with the atoms to which they are attached, form a 5- or 6-membered ring that is optionally containing one or two heteroatoms selected from O, N, and S; wherein the 5- or 6-membered ring is saturated, unsaturated, or aromatic; and wherein the 5- or 6-membered ring is optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³;
each R⁵ is independently selected from a group consisting of halogen and C₁₋₆alkyl;
R^{5a} is selected from the group consisting of hydrogen and C₁-C₆alkyl;
R²⁵ is selected from a group consisting of halogen, —CN, —OH, —OR⁶, —SR⁶, —S(═O)R⁷, —NO₂, —N(R⁶)₂, —S(═O)₂R⁷, —NHS(═O)₂R⁷, —S(═O)₂N(R⁶)₂, —C(═O)R⁷, —C(═O)OR⁶, —OC(═O)R⁷, —C(═O)N(R⁶)₂, —OC(═O)N(R⁶)₂, —NR⁶C(═O)N(R⁶)₂, —NR⁶C(═O)R⁷, —NR⁶C(═O)OR⁶, C₁₋₆alkyl, C₂₋₆alkenyl, C₂₋₆alkynyl, C₃₋₈cycloalkyl, C₂₋₉heterocycle, C₆₋₁₀aryl, C₁₋₉heteroaryl, and a fused C₅₋₉heteroaryl-cycloalkyl; wherein C₁₋₆alkyl, C₂₋₆alkenyl, C₂₋₆alkynyl, C₃₋₈cycloalkyl, C₂₋₉heterocycle, C₆₋₁₀aryl, C₁₋₉heteroaryl, and fused C₅₋₉heteroaryl-cycloalkyl are optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³;
each R⁶ is independently selected from the group consisting of hydrogen, C₁-C₆alkyl, —C₁-C₆alkyl-O—C₁-C₆alkyl, —C₁-C₆alkyl-C₂₋₉heterocycle, —C₁-C₆alkyl-C₂₋₉heteroaryl, C₃-C₈cycloalkyl, and C₂₋₉heterocycle; or two R⁶ on the same heteroatom are taken together with that heteroatom to which they are attached to form a C₂₋₉heterocycle or a C₂₋₉heteroaryl;
each R⁷ is independently selected from the group consisting of C₁-C₆alkyl and C₃-C₈cycloalkyl;
R⁸ is selected from the group consisting of hydrogen and C₁-C₆alkyl;
each R⁹ is independently selected from the group consisting of hydrogen, halogen, and C₁-C₆alkyl;
each R¹³ is independently selected from the group consisting of hydrogen, C₁-C₆alkyl, and C₃-C₈cycloalkyl; or two R¹³ on the same heteroatom are taken together with that heteroatom to which they are attached to form a C₂₋₉heterocycle;
each R¹⁴ is independently selected from the group consisting of C₁-C₆alkyl and C₃-C₈cycloalkyl;
n is 0, 1, 2, 3, or 4;
p is 0, 1, 2, or 3; and
q is 0, 1, or 2.

In some embodiments described herein is a method for treating an organ disease or disorder in a subject in need thereof comprising administering to the subject a therapeutically effective amount of a compound of Formula III, or a pharmaceutically acceptable salt or solvate thereof:

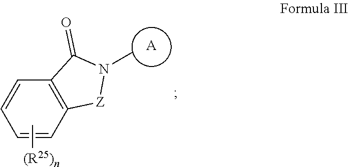

wherein

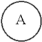

is

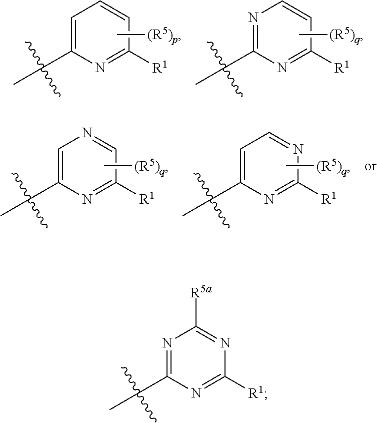

R¹ is

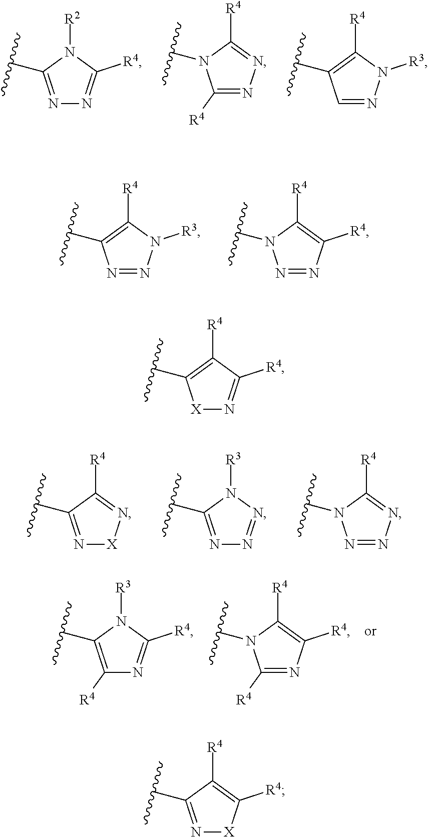

Z is O, S, C(═O), N(R⁸), or C(R⁹)₂;
X is O or S;
R² is C₃₋₆cycloalkyl;
R³ is selected from a group consisting of hydrogen, C₁₋₆alkyl, and C₃₋₆cycloalkyl;
each R⁴ is independently selected from a group consisting of hydrogen, halo, C₁₋₆alkyl, and C₃₋₆cycloalkyl;
   or one R⁴ and another R², R³, or R⁴, together with the atoms to which they are attached, form a 5- or 6-membered ring that is optionally containing one or two heteroatoms selected from O, N, and S; wherein the 5- or 6-membered ring is saturated, unsaturated, or aromatic; and wherein the 5- or 6-membered ring is optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³;
each R⁵ is independently selected from a group consisting of halogen and C₁₋₆alkyl;
R^{5a} is selected from the group consisting of hydrogen and C₁-C₆alkyl;
each R²⁵ is independently selected from a group consisting of halogen, —CN, —OH, —OR⁶, —SR⁶, —S(═O)R⁷, —NO₂, —N(R⁶)₂, —S(═O)₂R⁷, —NHS(═O)₂R⁷, —S(═O)₂N(R⁶)₂, —C(═O)R⁷, —C(═O)OR⁶, —OC(═O)R⁷, —C(═O)N(R⁶)₂, —OC(═O)N(R⁶)₂, —NR⁶C(═O)N(R⁶)₂, —NR⁶C(═O)R⁷, —NR⁶C(═O)OR⁶, C₁₋₆alkyl, C₂₋₆alkenyl, C₂₋₆alkynyl, C₃₋₈cycloalkyl, C₂₋₉heterocycle, C₆₋₁₀aryl, C₁₋₉heteroaryl, and a fused C₅₋₉heteroaryl-cycloalkyl; wherein C₁₋₆alkyl, C₂₋₆alkenyl, C₂₋₆alkynyl, C₃₋₈cycloalkyl, C₂₋₉heterocycle, C₆₋₁₀aryl, C₁₋₉heteroaryl, and fused C₅₋₉heteroaryl-cycloalkyl are optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³;
each R⁶ is independently selected from the group consisting of hydrogen, C₁-C₆alkyl, —C₁-C₆alkyl-O—C₁-C₆alkyl, —C₁-C₆alkyl-C₂₋₉heterocycle, —C₁-C₆alkyl-C₂₋₉heteroaryl, C₃-C₈cycloalkyl, —C₃-C₈cycloalkyl-phenyl, and C₂₋₉heterocycle, wherein C₁-C₆alkyl, —C₁-C₆alkyl-O—C₁-C₆alkyl, —C₁-C₆alkyl-C₂₋₉heterocycle, —C₁-C₆alkyl-C₂₋₉heteroaryl, C₃-C₈cycloalkyl, —C₃-C₈cycloalkyl-phenyl, and C₂₋₉heterocycle are optionally substituted with one, two, or three substituents selected from the group consisting of halo, —OR⁸, —SR⁸, —N(R⁸)₂, —C₁₋₆alkyl, —O—C₁₋₆alkyl, —C(═O)R¹⁴, —C(═O)OR¹³, and —N(R¹³)C(═O)R¹⁴; or two R⁶ on the same heteroatom are taken together with that heteroatom to which they are attached to form a C₂₋₉heterocycle or a C₂₋₉heteroaryl, wherein C₂₋₉heterocycle or C₂₋₉heteroaryl are optionally substituted with one, two, or three substituents selected from the group consisting of halo, —OR⁸, —SR⁸, —N(R⁸)₂, —C₁₋₆alkyl, —O—C₁₋₆alkyl, —C(═O)R¹⁴, —C(═O)OR¹³, and —N(R¹³)C(═O)R¹⁴;
each R⁷ is independently selected from the group consisting of C₁-C₆alkyl, C₃-C₈cycloalkyl, and C₂₋₉heterocycle, wherein C₃-C₈cycloalkyl and C₂₋₉heterocycle are optionally substituted with one, two, or three substituents selected from the group consisting of halo, oxo, —OR, —SR⁸, —N(R⁸)₂, —C₁₋₆alkyl, —O—C₁₋₆alkyl, —C(═O)R¹⁴, —C(═O)OR¹³, and —N(R¹³)C(═O)R¹⁴;
each R⁸ is independently selected from the group consisting of hydrogen and C₁-C₆alkyl;
each R⁹ is independently selected from the group consisting of hydrogen, halogen, and C₁-C₆alkyl;
each R¹³ is independently selected from the group consisting of hydrogen, C₁-C₆alkyl, and C₃-C₈cycloalkyl; or two R¹³ on the same heteroatom are taken together with that heteroatom to which they are attached to form a C₂₋₉heterocycle;
each R¹⁴ is independently selected from the group consisting of C₁-C₆alkyl and C₃-C₈cycloalkyl;
n is 0, 1, 2, 3, or 4;
p is 0, 1, 2, or 3; and
q is 0, 1, or 2.

In some embodiments, R²⁵ is selected from a group consisting of C₃₋₈cycloalkyl, C₂₋₉heterocycle, C₆₋₁₀aryl, C₁₋₉heteroaryl, and a fused C₅₋₉heteroaryl-cycloalkyl; wherein C₃₋₈cycloalkyl, C₂₋₉heterocycle, C₆₋₁₀aryl, C₁₋₉heteroaryl, and fused C₅₋₉heteroaryl-cycloalkyl, wherein C₃₋₈cycloalkyl, C₂₋₉heterocycle, C₆₋₁₀aryl, C₁₋₉heteroaryl, and fused C₅₋₉heteroaryl-cycloalkyl are optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R²⁵ is selected from a group consisting of C₂₋₉heterocycle and C₁₋₉heteroaryl; wherein C₂₋₉heterocycle and C₁₋₉heteroaryl are optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R²⁵ is selected from a group consisting of C₂₋₉heterocycle and C₁₋₉heteroaryl; wherein C₂₋₉heterocycle and C₁₋₉heteroaryl are optionally substituted with one or two substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R²⁵ is selected from a group consisting of pyrazole, imidazole, thiazole, and pyridine; wherein pyrazole, imidazole, thiazole, and pyridine are optionally substituted with one or two substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R²⁵ is selected from a group consisting of pyrazole, imidazole, thiazole, and pyridine; wherein pyrazole, imidazole, thiazole, and pyridine are optionally substituted with one or two substituents selected from the group consisting of halo, C₁₋₆alkyl, and C₃₋₈cycloalkyl.

In some embodiments, R²⁵ is

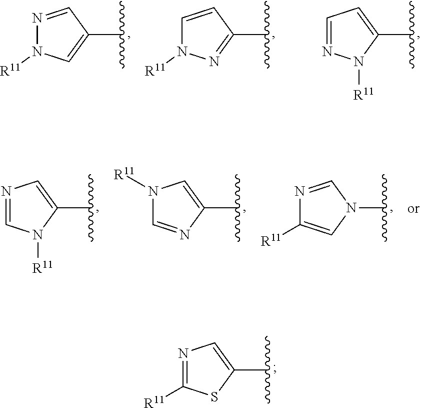

wherein each R¹¹ is independently C₁-C₆alkyl or C₃-C₆cycloalkyl.

In some embodiments, R²⁵ is

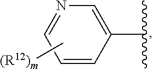

wherein each R¹² is independently hydrogen, halo, C₁-C₆alkyl, or C₃-C₆cycloalkyl; and m is 1 or 2.

In some embodiments, R²⁵ is selected from a group consisting of unsubstituted pyrazole, unsubstituted imidazole, unsubstituted thiazole, and unsubstituted pyridine.

In some embodiments, R²⁵ is selected from a group consisting of pyrimidine, pyrazine, and pyridazine; wherein pyrimidine, pyrazine, and pyridazine are optionally substituted with one or two substituents selected from the group consisting of halo, C₁₋₆alkyl, and C₃₋₈cycloalkyl.

In some embodiments, R²⁵ is selected from a group consisting of halogen, —OR⁶, —N(R⁶)₂, C₁₋₆alkyl, pyrazole, imidazole, thiazole, and pyridine; wherein pyrazole, imidazole, thiazole, and pyridine are optionally substituted with one or two substituents selected from the group consisting of halo, C₁₋₆alkyl, and C₃₋₈cycloalkyl.

In some embodiments, R²⁵ is selected from a group consisting of halogen, —OR⁶, —N(R⁶)₂, C₁₋₆alkyl, and unsubstituted pyridine.

In some embodiments, R²⁵ is —C(═O)N(R⁶)₂ and each R⁶ is independently selected from the group consisting of hydrogen, C₁-C₆alkyl, —C₁-C₆alkyl-O—C₁-C₆alkyl, —C₁-C₆alkyl-C₂₋₉heterocycle, —C₁-C₆alkyl-C₂₋₉heteroaryl, C₃-C₈cycloalkyl, and C₂₋₉heterocycle.

In some embodiments, R²⁵ is

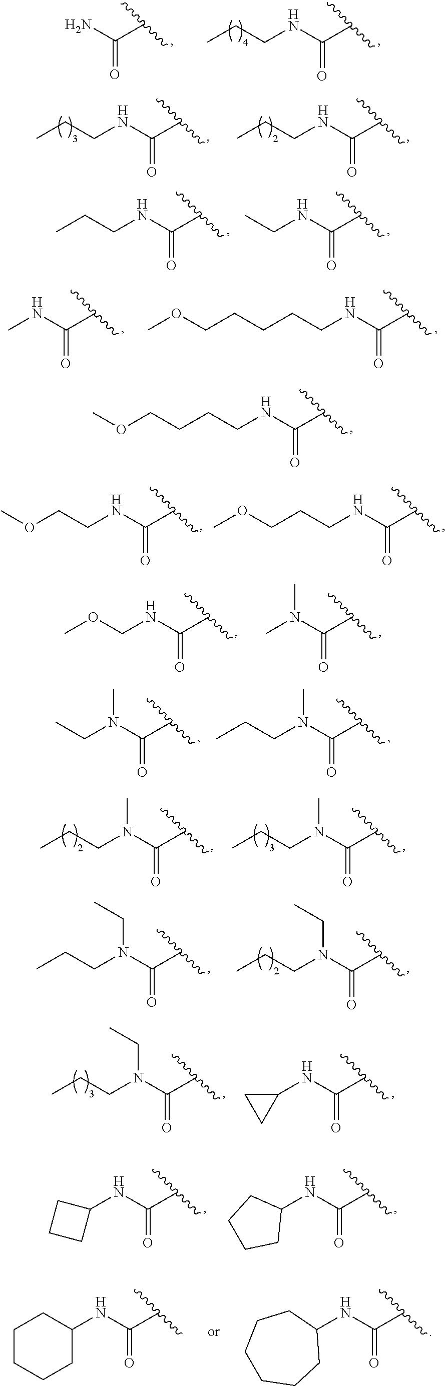

In some embodiments, R²⁵ is

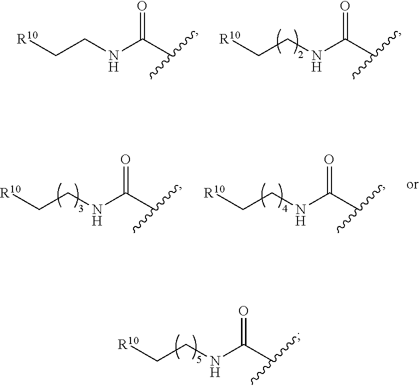

wherein R¹⁰ is a heteroaryl.

In some embodiments, R²⁵ is —C(═O)N(R⁶)₂ and two R⁶ on the same heteroatom are taken together with that heteroatom to which they are attached to form a C₂₋₉heterocycle or a C₂₋₉heteroaryl, wherein C₂₋₉heterocycle or C₂₋₉heteroaryl are optionally substituted with one, two, or three substituents selected from the group consisting of halo, —OR⁸, —SR⁸, —N(R⁸)₂, —C₁₋₆alkyl, —O—C₁₋₆alkyl, —C(═O)R¹⁴, —C(═O)OR¹³, and —N(R¹³)C(═O)R¹⁴.

In some embodiments, R²⁵ is

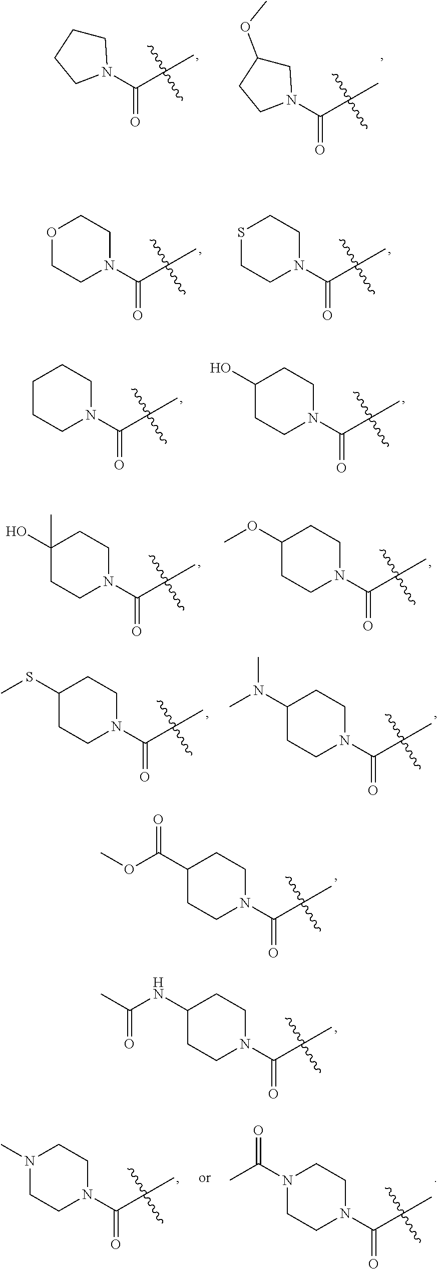

In some embodiments, R²⁵ is —C(═O)N(R⁶)₂ and two R⁶ on the same heteroatom are taken together with that heteroatom to which they are attached to form a C₂₋₉heterocycle or a C₂₋₉heteroaryl.

In some embodiments, R²⁵ is —C(═O)N(R⁶)₂ and two R⁶ are taken together with that heteroatom to which they are attached to form a C₂₋₉heterocycle or a C₂₋₉heteroaryl.

In some embodiments, R²⁵ is

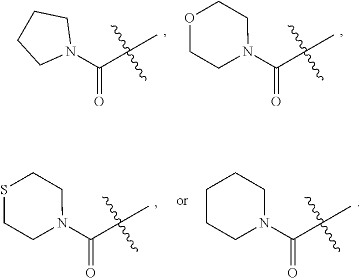

In some embodiments, R²⁵ is —OR⁶ and R⁶ is selected from the group consisting of C₁-C₆alkyl, —C₁-C₆alkyl-O—C₁-C₆alkyl, and —C₁-C₆alkyl-C₂₋₉heterocycle.

In some embodiments, R¹ is

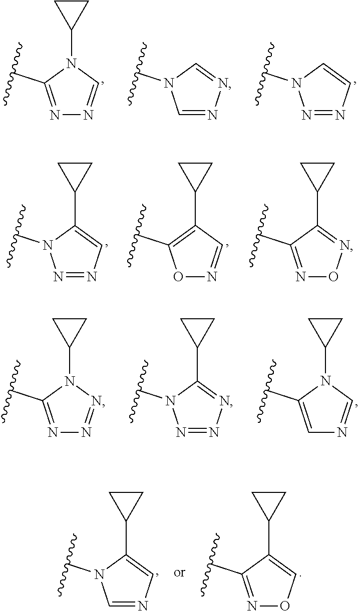

In some embodiments, R¹ is

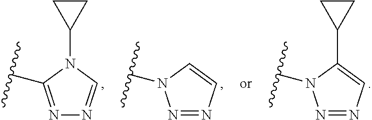

In some embodiments, R¹ is

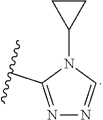 [SMILES: *c1nncn1C1CC1]

In some embodiments, R¹ is

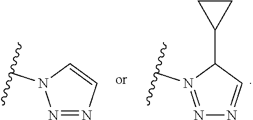

In some embodiments, R¹ is

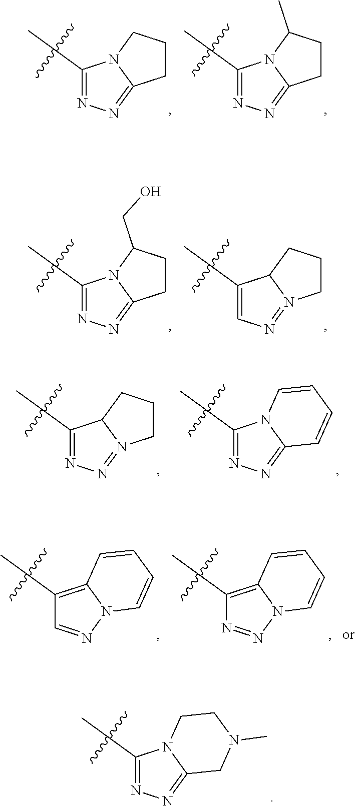

In some embodiments,

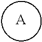

is

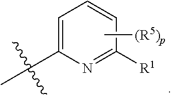

In some embodiments, p is 0.

In some embodiments,

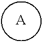

is

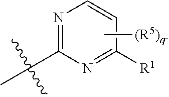

In some embodiments,

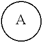

is

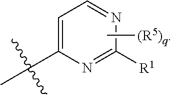

In some embodiments, q is 0.

In some embodiments, n is 0. In some embodiments, n is 1. In some embodiments, n is 2. In some embodiments, Z is C(R⁹)₂. In some embodiments, R⁹ is H.

In a further aspect described herein are pharmaceutical compositions comprising a compound of Formula I, Formula II, Formula IIa, or Formula III, or a pharmaceutically acceptable salt or solvate thereof, and at least one pharmaceutically acceptable excipient.

In another aspect described herein are methods of treating an organ disease or disorder in a subject in need thereof, comprising administering to the subject a therapeutically effective amount of a compound of Formula I, Formula II, Formula IIa, or Formula III, or a pharmaceutically acceptable salt or solvate thereof. In another aspect described herein are methods of treating an organ disease or disorder in a subject in need thereof, comprising administering to the subject a therapeutically effective amount of a pharmaceutical composition comprising a compound of Formula I, Formula II, Formula IIa, or Formula III, or a pharmaceutically acceptable salt or solvate thereof, and at least one pharmaceutically acceptable excipient.

In some embodiments described herein are methods of treating an organ disease or disorder in a subject in need thereof, comprising administering to the subject a therapeutically effective amount of a compound of Formula I, Formula II, Formula IIa, or Formula III, or a pharmaceutically acceptable salt or solvate thereof, wherein the organ disease or disorder is selected from liver disease or disorder, heart disease or disorder, kidney disease or disorder, pancreas disease or disorder, spleen disease or disorder, and skin disease or disorder. In some embodiments described herein are methods of treating an organ disease or disorder in a subject in need thereof, comprising administering to the subject a therapeutically effective amount of a compound of Formula I, Formula II, Formula IIa, or Formula III, or a pharmaceutically acceptable salt or solvate thereof, wherein the organ disease or disorder is selected from liver disease or disorder, heart disease or disorder, and kidney disease or disorder. In some embodiments described herein are methods of treating an organ disease or disorder in a subject in need thereof, comprising administering to the subject a therapeutically effective amount of a compound of Formula I, Formula II, Formula IIa, or Formula III, or a pharmaceutically acceptable salt or solvate thereof, wherein the organ disease or disorder is a liver disease or disorder. In some embodiments described herein are methods of treating an organ disease or disorder in a subject in need thereof, comprising administering to the subject a therapeutically effective amount of a compound of Formula I, Formula II, Formula IIa, or Formula III, or a pharmaceutically acceptable salt or solvate thereof, wherein the organ disease or disorder is a liver disease or disorder selected from a liver injury, a drug-induced liver injury, a liver failure, a fulminant or acute liver failure and an acute-on-chronic liver failure. Examples of fulminant liver failure comprise fulminant liver failure due to a drug overdose, for example a acetaminophen overdose, fulminant liver failure due to poisoning with wild mushroom Amanita phalloides or carbon tetrachloride disorder. Examples of acute liver failure comprise acute liver failure due to poisoning with e.g. herbal supplements, acute liver failure due to surgical ischemia-reperfusion injury (liver transplant, partial resection), acute liver failure due to vascular diseases, acute liver failure due to metabolic disease, acute fatty liver of pregnancy, acute liver failure due to septic shock. Examples of acute-on-chronic failure comprise acute-on-chronic liver failure due to alcoholic hepatitis, acute-on-chronic liver failure due to autoimmune hepatitis, acute-on-chronic liver failure due to viral hepatitis, and acute-on-chronic liver failure due to cirrhosis. In some embodiments described herein are methods of treating an organ disease or disorder in a subject in need thereof, comprising administering to the subject a therapeutically effective amount of a compound of Formula I, Formula II, Formula IIa, or Formula III, or a pharmaceutically acceptable salt or solvate thereof, wherein the organ disease or disorder is a heart disease or disorder. In some embodiments described herein are methods of treating an organ disease or disorder in a subject in need thereof, comprising administering to the subject a therapeutically effective amount of a compound of Formula I, Formula II, Formula IIa, or Formula III, or a pharmaceutically acceptable salt or solvate thereof, wherein the organ disease or disorder is a heart disease or disorder selected from ischemia reperfusion injury, ischemia, coronary artery disease, cardiovascular dysfunction from sepsis, drug-induced cardiotoxicity, viral myocarditis, and complications due to heart transplant. In some embodiments described herein are methods of treating an organ disease or disorder in a subject in need thereof, comprising administering to the subject a therapeutically effective amount of a compound of Formula I, Formula II, Formula IIa, or Formula III, or a pharmaceutically acceptable salt or solvate thereof, wherein the organ disease or disorder is a kidney disease or disorder. In some embodiments described herein are methods of treating an organ disease or disorder in a subject in need thereof, comprising administering to the subject a therapeutically effective amount of a compound of Formula I, Formula II, Formula IIa, or Formula III, or a pharmaceutically acceptable salt or solvate thereof, wherein the organ disease or disorder is a kidney disease or disorder is an acute kidney injury. In some embodiments described herein are methods of treating an organ disease or disorder in a subject in need thereof, comprising administering to the subject a therapeutically effective amount of a compound of Formula I, Formula II, Formula IIa, or Formula III, or a pharmaceutically acceptable salt or solvate thereof, wherein the organ disease or disorder is a kidney disease or disorder is an acute kidney injury due to hypovolemia, hypotension, renal vasoconstriction, glomerular efferent arteriolar vasodilation, prolonged renal ischemia, prolonged renal sepsis, prolonged renal nephrotoxins, kidney transplants, acute tubular necrosis, acute interstitial nephritis, glomerulonephritis, or intratubular obstruction.

In some embodiments of the methods described herein, a compound of Formula I, Formula II, Formula IIa, or Formula III, or a pharmaceutically acceptable salt or solvate thereof, is administered by injectable delivery. In some embodiments of the methods described herein, a compound of Formula I, Formula II, Formula IIa, or Formula III, or a pharmaceutically acceptable salt or solvate thereof, is administered by intravenous delivery, wherein the intravenous delivery is selected from bolus injection, intravenous drip, and infusion pump. In some embodiments of the methods described herein, a compound of Formula I, Formula II, Formula IIa, or Formula III, or a pharmaceutically acceptable salt or solvate thereof, is administered by intraperitoneal delivery.

In some embodiments, the methods described herein further comprise the administration of a second therapeutic agent. In some embodiments, the second therapeutic agent is selected from N-acetylcysteine, corticosteroids, and vasopressors.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### DETAILED DESCRIPTION OF THE INVENTION

ASK1 is a membrane-proximal MAP3K (MAP-kinase-kinase-kinase) upstream of pathways that play important roles in the cellular response to environmental stresses including ischemia and inflammation. The downstream ASK1 cascade includes the c-Jun and p38 pathways, that are known to be responsive to UV and oxidative damage and result in apoptosis and inflammation. ASK1 is a promising therapeutic target for organ diseases, including diseases of liver injury, kidney injury, and heart diseases. A positive regulator of mitochondrial apoptosis, ASK1 is tightly regulated and activated by cellular damage signals as diverse as receptor-acting inflammatory cytokines (e.g., TNFa and LPS), calcium and intracellular sensors (e.g., the redox sensor thioredoxin, and the ER-stress-responsive IRE1).

ASK1 regulation is a promising therapeutic target for liver injury. Liver injuries include but is not limited to drug-induced liver injury, fulminant liver failure, acute liver failure, and acute-on-chronic liver failure. Drug-induced liver failure includes, but is not limited to antibiotics, nonsteroidal anti-inflammatory drugs, and anti-convulsant drugs leading to liver failure. Fulminant liver failure can be due to a variety of causes including, but not limited to acetaminophen overdose and poisoning. Poisoning can be with wild mushroom Amanita phalloides, carbon tetrachloride, and herbal supplements including but not limited to kava, ephedra, skullcap, and pennyroyal. Acute liver failure can be due to surgical ischemia-reperfusion injury (liver transplant, partial resection), vascular diseases, such as but not limited to Budd-Chiari syndrome, metabolic diseases such as but not limited to Wilson's disease and acute fatty liver of pregnancy, and septic shock. Acute-on-chronic liver failure can be due to a variety of causes including, but not limited to alcoholic hepatitis, autoimmune hepatitis, and viral hepatitis including but not limited to hepatitis A, hepatitis B, and hepatitis E, Epstein Barr virus, cytomegalovirus, and herpes simplex virus.

ASK1 regulation is a promising therapeutic target for acute kidney injury. Acute kidney injury can be due to a variety of causes including, but not limited to hypovolemia, hypotension, renal vasoconstriction, glomerular efferent arteriolar vasodilation, prolonged renal ischemia, kidney transplants, acute tubular necrosis, acute interstitial nephritis, glomerulonephritis, and intratubular obstruction.

ASK1 regulation is a promising therapeutic target for heart diseases and disorders. Heart diseases and disorders include but is not limited to ischemia reperfusion injury, ischemia, septic shock, cardiac toxicity, and viral myocarditis, or diseases and disorders due to heart transplant.

In some embodiments, ASK1 inhibitors described herein are delivered orally or by injection. ASK1 inhibitors described herein show preferential distribution to liver and kidney after oral or injection and achieve organ concentration several fold above the plasma concentration. In some embodiments, injectable dosing is advantageous for treatment of patients in whom the injectable route of administration is preferred over the oral route, including patients who are unconscious, have swallowing difficulties or require a rapid onset of pharmacological activity.

Further, in some embodiments, the ASK1 inhibitors described herein are useful as an adjuvant for systematic treatment with N-acetylcysteine, corticosteroids, and vasopressors such as but not including vasopressin and catecholamines.

### Certain Terminology

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. Unless otherwise indicated, conventional methods of mass spectroscopy, NMR, HPLC, protein chemistry, biochemistry, recombinant DNA techniques and pharmacology are employed. In this application, the use of “or” or “and” means “and/or” unless stated otherwise. Furthermore, use of the term “including” as well as other forms, such as “include”, “includes,” and “included,” is not limiting. The section headings used herein are for organizational purposes only and are not to be construed as limiting the subject matter described.

An “alkyl” group refers to an aliphatic hydrocarbon group. The alkyl moiety may be branched or straight chain. The “alkyl” group may have 1 to 15 carbon atoms (whenever it appears herein, a numerical range such as “1 to 15” refers to each integer in the given range; e.g., “1 to 15 carbon atoms” means that the alkyl group may consist of 1 carbon atom, 2 carbon atoms, 3 carbon atoms, etc., up to and including 15 carbon atoms, although the present definition also covers the occurrence of the term “alkyl” where no numerical range is designated). In one aspect the alkyl is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, sec-butyl, tertiary butyl, pentyl, neopentyl, hexyl, and the like.

The term “alkenyl” refers to a type of alkyl group in which at least one carbon-carbon double bond is present. In one embodiment, an alkenyl group has the formula —C(R)═CR₂, wherein R refers to the remaining portions of the alkenyl group, which may be the same or different. In some embodiments, R is H or an alkyl. Non-limiting examples of an alkenyl group include —CH═CH₂, —C(CH₃)═CH₂, —CH═CHCH₃, —C(CH₃)═CHCH₃, and —CH₂CH═CH₂.

The term “alkynyl” refers to a type of alkyl group in which at least one carbon-carbon triple bond is present. In one embodiment, an alkynyl group has the formula —C≡C—R, wherein R refers to the remaining portions of the alkynyl group. In some embodiments, R is H or an alkyl. Non-limiting examples of an alkynyl group include —C≡CH, —C≡CCH₃—C≡CCH₂CH₃, —CH₂C≡CH.

The term “cycloalkyl” refers to a monocyclic or polycyclic aliphatic, non-aromatic radical, wherein each of the atoms forming the ring (i.e. skeletal atoms) is a carbon atom. Cycloalkyls may be saturated, or partially unsaturated. Cycloalkyls may be fused with an aromatic ring, and the point of attachment is at a carbon that is not an aromatic ring carbon atom. Cycloalkyl groups include groups having from 3 to 10 ring atoms. In some embodiments, cycloalkyl groups are selected from among cyclopropyl, cyclobutyl, cyclopentyl, cyclopentenyl, cyclohexyl, cyclohexenyl, cycloheptyl, and cyclooctyl. Cycloalkyl groups may be substituted or unsubstituted. Depending on the structure, a cycloalkyl group can be a monoradical or a diradical (i.e., an cycloalkylene group, such as, but not limited to, cyclopropan-1,1-diyl, cyclobutan-1,1-diyl, cyclopentan-1,1-diyl, cyclohexan-1,1-diyl, cyclohexan-1,4-diyl, cycloheptan-1,1-diyl, and the like). In one aspect, a cycloalkyl is a C₃-C₆cycloalkyl.

The term “aromatic” refers to a planar ring having a delocalized π-electron system containing 4n+2 π L electrons, where n is an integer. Aromatics are optionally substituted. The term “aromatic” includes both cycloalkyl aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups. The term “aryl” refers to an aromatic ring wherein each of the atoms forming the ring is a carbon atom. Aryl groups are optionally substituted. Depending on the structure, an aryl group can be a monoradical or a diradical (i.e., an arylene group).

The terms “heteroaryl” or, alternatively, “heteroaromatic” refers to an aryl group that includes one or more ring heteroatoms selected from nitrogen, oxygen and sulfur. Illustrative examples of heteroaryl groups include the following moieties:

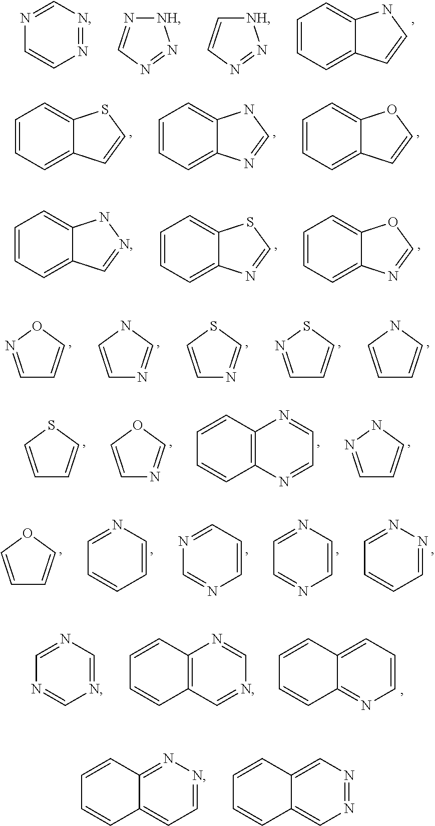

and the like. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. In some embodiments, a heteroaryl contains 0-3 N atoms in the ring. In some embodiments, a heteroaryl contains 1-3 N atoms in the ring. In some embodiments, a heteroaryl contains 0-3 N atoms, 0-1 O atoms, and 0-1 S atoms in the ring. In some embodiments, a heteroaryl is a monocyclic or bicyclic heteroaryl. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, bicyclic heteroaryl is a C₆-C₉heteroaryl. Depending on the structure, a heteroaryl group can be a monoradical or a diradical (i.e., a heteroarylene group).

A “heterocycle” or “heterocycloalkyl” group refers to a cycloalkyl group wherein at least one of the carbon atoms of the cycloalkyl is replaced with nitrogen (unsubstituted or substituted, e.g. —NH—, —N(alkyl)-), oxygen (—O—), or sulfur (e.g. —S—, —S(═O)— or —S(═O)₂—). The radicals may be fused with an aryl or heteroaryl. In some embodiments, the heterocycloalkyl is selected from oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, and indolinyl. The term heteroalicyclic also includes all ring forms of the carbohydrates, including but not limited to the monosaccharides, the disaccharides and the oligosaccharides. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl contains 0-3 N atoms in the ring. In some embodiments, a heterocycloalkyl contains 0-3 N atoms, 0-3 O atoms and 0-1 S atoms in the ring.

The term “halo” or, alternatively, “halogen” or “halide” means fluoro (F), chloro (Cl), bromo (Br) or iodo (I). The term “bond” or “single bond” refers to a chemical bond between two atoms, or two moieties when the atoms joined by the bond are considered to be part of larger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

The term “optionally substituted” or “substituted” means that the referenced group may be substituted with one or more additional group(s) individually and independently selected from alkyl, cycloalkyl, aryl, heteroaryl, heteroalicyclic, hydroxy, alkoxy, aryloxy, alkylthio, arylthio, alkylsulfoxide, arylsulfoxide, alkylsulfone, arylsulfone, cyano, halo, nitro, haloalkyl, fluoroalkyl, fluoroalkoxy, and amino, including mono- and di-substituted amino groups, and the protected derivatives thereof. In some embodiments, optional substituents are independently selected from halogen, —CN, —NH₂, —NH(CH₃), —N(CH₃)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some embodiments, optional substituents are independently selected from halogen, —CN, —NH₂, —OH, —NH(CH₃), —N(CH₃)₂, —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic, saturated or unsaturated carbon atoms, excluding aromatic carbon atoms) includes oxo (═O).

In certain embodiments, the compounds presented herein possess one or more stereocenters and each center independently exists in either the R or S configuration. The compounds presented herein include all diastereomeric, enantiomeric, and epimeric forms as well as the appropriate mixtures thereof. Stereoisomers are obtained, if desired, by methods such as, stereoselective synthesis and/or the separation of stereoisomers by chiral chromatographic columns. In some embodiments, halogen is F or Cl. In some embodiments, halogen is F.

The methods and formulations described herein include the use of N-oxides (if appropriate), crystalline forms (also known as polymorphs), or pharmaceutically acceptable salts of compounds having the structure of Formula I, Formula II, Formula IIa, or Formula III, as well as active metabolites of these compounds having the same type of activity. In some situations, compounds may exist as tautomers. All tautomers are included within the scope of the compounds presented herein. In specific embodiments, the compounds described herein exist in solvated forms with pharmaceutically acceptable solvents such as water, ethanol, and the like. In other embodiments, the compounds described herein exist in unsolvated form.

The term “acceptable” with respect to a formulation, composition or ingredient, as used herein, means having no persistent detrimental effect on the general health of the subject being treated.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound of Formula I, Formula II, Formula IIa, or Formula III, or a pharmaceutically acceptable salt thereof, and a co-agent, are both administered to a patient simultaneously in the form of a single entity or dosage. The term “non-fixed combination” means that the active ingredients, e.g. a compound of Formula I, Formula II, Formula IIa, or Formula III, or a pharmaceutically acceptable salt thereof, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Methods

In some embodiments described herein is a method for treating an organ disease or disorder in a subject in need thereof comprising administering to the subject a therapeutically effective amount of a compound of Formula I, or a pharmaceutically acceptable salt or solvate thereof:

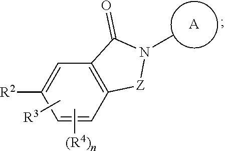

wherein

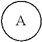

is

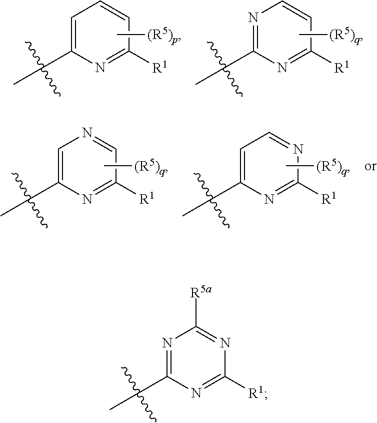

Z is O, S, C(═O), N(R⁸), or C(R⁹)₂;
R¹ and R³ are each independently selected from a group consisting of hydrogen, halogen, —CN, —OH, —OR⁶, —SR⁶, —S(═O)R⁷, —NO₂, —N(R⁶)₂, —S(═O)₂R⁷, —NHS(═O)₂R⁷, —S(═O)₂N(R⁶)₂, —C(═O)R⁷, —C(═O)OR⁶, —OC(═O)R⁷, —C(═O)N(R⁶)₂, —OC(═O)N(R⁶)₂, —NR⁶C(═O)N(R⁶)₂, —NR⁶C(═O)R⁷, —NR⁶C(═O)OR⁶, C₁₋₆alkyl, C₂₋₆alkenyl, C₂₋₆alkynyl, C₃₋₈cycloalkyl, C₂₋₉heterocycle, C₆₋₁₀aryl, C₁₋₉heteroaryl, and a fused C₅₋₉heteroaryl-cycloalkyl; wherein C₁₋₆alkyl, C₂₋₆alkenyl, C₂₋₆alkynyl, C₃₋₈cycloalkyl, C₂₋₉heterocycle, C₆₋₁₀aryl, C₁₋₉heteroaryl, and fused C₅₋₉heteroaryl-cycloalkyl are optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³;
R² is selected from a group consisting of hydrogen, halogen, —CN, —OH, —SR⁶, —S(═O)R⁷, —NO₂, —N(R⁶)₂, —S(═O)₂R⁷, —NHS(═O)₂R⁷, —S(═O)₂N(R⁶)₂, —C(═O)R⁷, —C(═O)OR⁶, —OC(═O)R⁷, —C(═O)N(R⁶)₂, —OC(═O)N(R⁶)₂, —NR⁶C(═O)N(R⁶)₂, —NR⁶C(═O)R⁷, —NR⁶C(═O)OR⁶, C₁₋₆alkyl, C₂₋₆alkenyl, C₂₋₆alkynyl, C₃₋₈cycloalkyl, C₂₋₉heterocycle, C₆₋₁₀aryl, C₁₋₉heteroaryl, and a fused C₅₋₉heteroaryl-cycloalkyl; wherein C₁₋₆alkyl, C₂₋₆alkenyl, C₂₋₆alkynyl, C₃₋₈cycloalkyl, C₂₋₉heterocycle, C₆₋₁₀aryl, C₁₋₉heteroaryl, and fused C₅₋₉heteroaryl-cycloalkyl are optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³; wherein R² and R³ are not both hydrogen;
each R⁴ and each R⁵ are each independently selected from a group consisting of halogen, —CN, and C₁₋₆alkyl;
R^{5a} is selected from the group consisting of hydrogen and C₁-C₆alkyl;
each R⁶ is independently selected from the group consisting of hydrogen, C₁-C₆alkyl, —C₁-C₆alkyl-O—C₁-C₆alkyl, —C₁-C₆alkyl-C₂₋₉heterocycle, —C₁-C₆alkyl-C₂₋₉heteroaryl, C₃-C₈cycloalkyl, and C₂₋₉heterocycle; or two R⁶ on the same heteroatom are taken together with that heteroatom to which they are attached to form a C₂₋₉heterocycle or a C₂₋₉heteroaryl;
each R⁷ is independently selected from the group consisting of C₁-C₆alkyl and C₃-C₈cycloalkyl;
R⁸ is selected from the group consisting of hydrogen and C₁-C₆alkyl;
each R⁹ is independently selected from the group consisting of hydrogen, halogen, and C₁-C₆alkyl; each R¹³ is independently selected from the group consisting of hydrogen, C₁-C₆alkyl, and C₃-C₈cycloalkyl; or two R¹³ on the same heteroatom are taken together with that heteroatom to which they are attached to form a C₂₋₉heterocycle;
each R¹⁴ is independently selected from the group consisting of C₁-C₆alkyl and C₃-C₈cycloalkyl;
n is 0, 1, or 2;
p is 0, 1, 2, or 3; and
q is 0, 1, or 2.

In some embodiments, R² is selected from a group consisting of C₃₋₈cycloalkyl, C₂₋₉heterocycle, C₆₋₁₀aryl, C₁₋₉heteroaryl, and a fused C₅₋₉heteroaryl-cycloalkyl; wherein C₃₋₈cycloalkyl, C₂₋₉heterocycle, C₆₋₁₀aryl, C₁₋₉heteroaryl, and fused C₅₋₉heteroaryl-cycloalkyl are optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R² is selected from a group consisting of C₂₋₉heterocycle and C₁₋₉heteroaryl; wherein C₂₋₉heterocycle and C₁₋₉heteroaryl are optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R² is selected from a group consisting of C₂₋₉heterocycle and C₁₋₉heteroaryl; wherein C₂₋₉heterocycle and C₁₋₉heteroaryl are optionally substituted with one or two substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R² is selected from a group consisting of pyrazole, imidazole, thiazole, and pyridine; wherein pyrazole, imidazole, thiazole, and pyridine are optionally substituted with one or two substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R² is selected from a group consisting of pyrazole, imidazole, thiazole, and pyridine; wherein pyrazole, imidazole, thiazole, and pyridine are optionally substituted with one or two substituents selected from the group consisting of halo, C₁₋₆alkyl, and C₃₋₈cycloalkyl.

In some embodiments, R² is

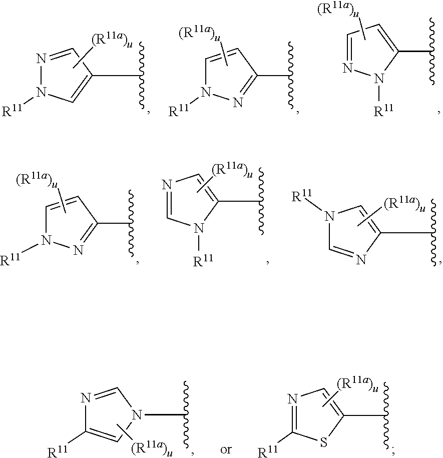

each R¹¹ is C₁-C₆alkyl or C₃-C₆cycloalkyl; R^{11a} is —CN, —OH, C₁-C₆alkyl, or C₃-C₆cycloalkyl; and u is 0, 1 or 2.

In some embodiments, R² is

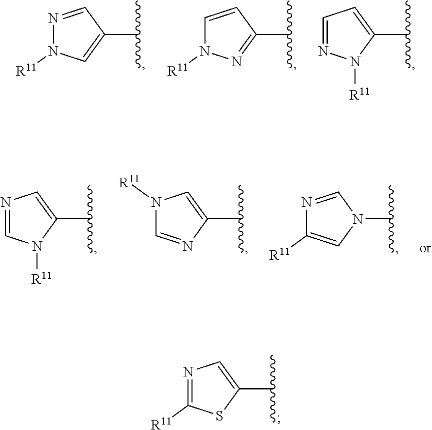

wherein R¹¹ is C₁-C₆alkyl or C₃-C₆cycloalkyl.

In some embodiments, R² is

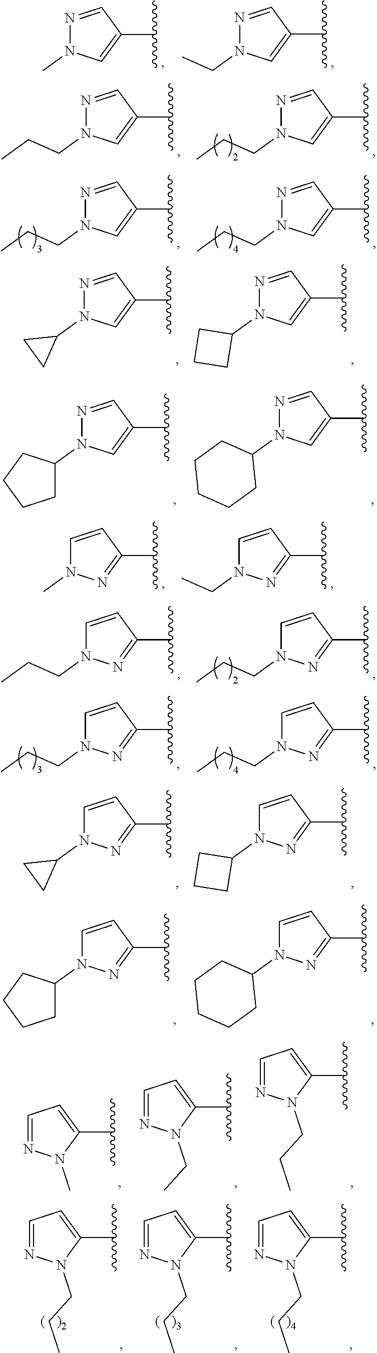

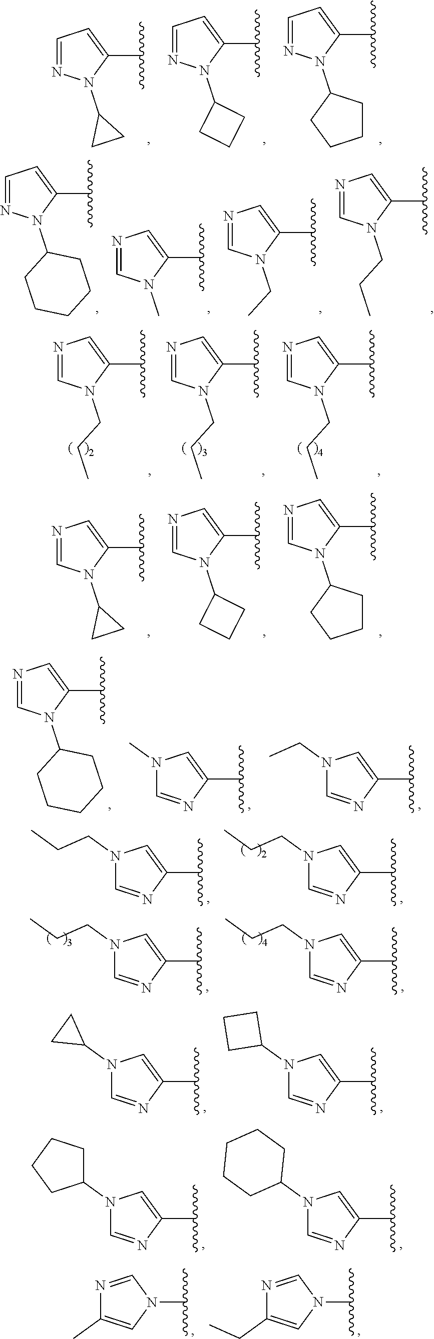

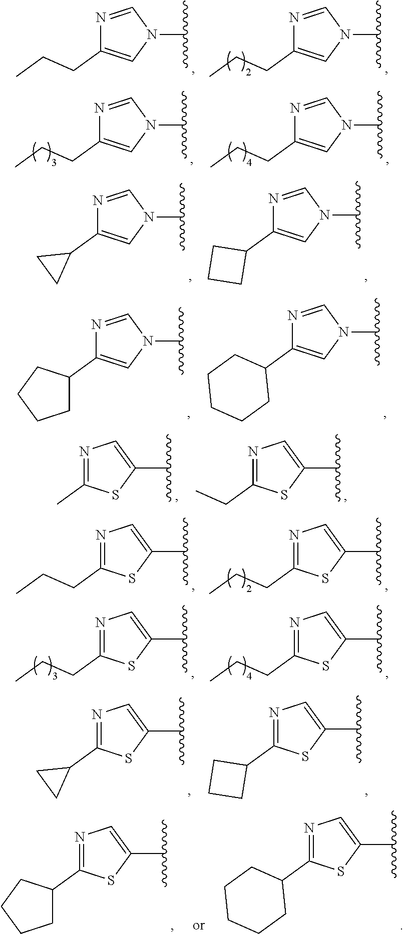

In some embodiments, R² is

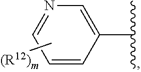

wherein each R¹² is independently halo, C₁-C₆alkyl, or C₃-C₆cycloalkyl; and m is 1 or 2.

In some embodiments, R² is selected from a group consisting of unsubstituted pyrazole, unsubstituted imidazole, unsubstituted thiazole, and unsubstituted pyridine.

In some embodiments, R² is —C(═O)N(R⁶)₂ and each R⁶ is independently selected from the group consisting of hydrogen, C₁-C₆alkyl, —C₁-C₆alkyl-O—C₁-C₆alkyl, —C₁-C₆alkyl-C₂₋₉heterocycle, —C₁-C₆alkyl-C₂₋₉heteroaryl, C₃-C₈cycloalkyl, and C₂₋₉heterocycle.

In some embodiments, R² is

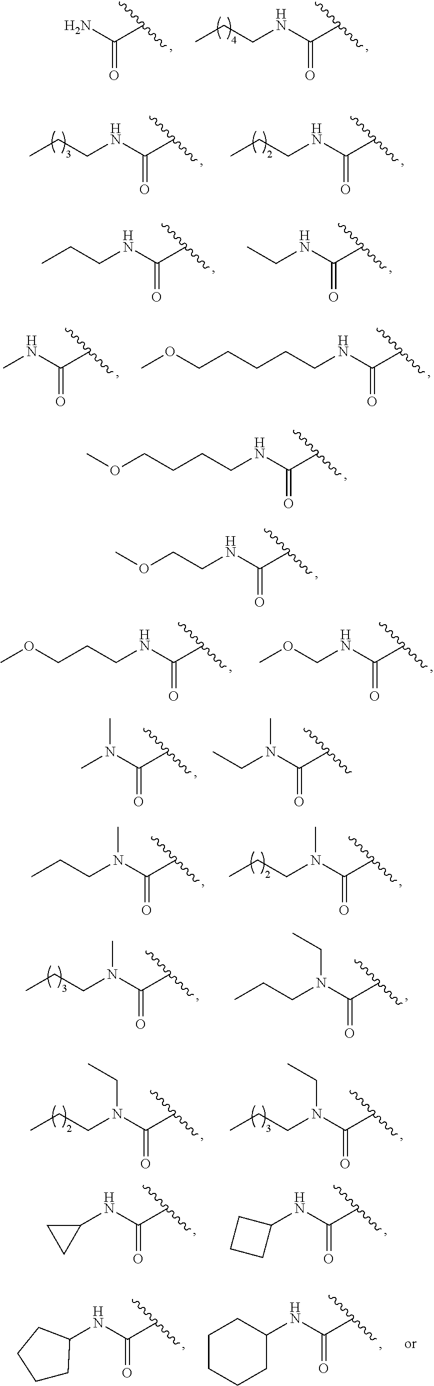

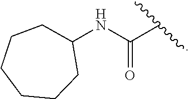 [SMILES: *C(=O)NC1CCCCCC1]

In some embodiments, R² is

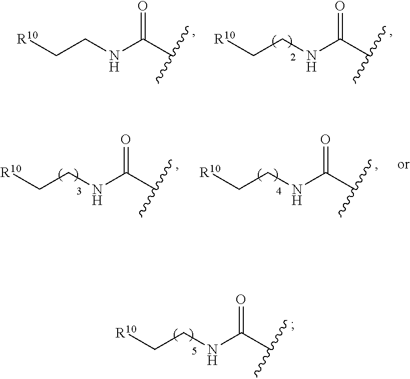

wherein R¹⁰ is a heteroaryl.

In some embodiments, R³ is hydrogen. In some embodiments, R³ is C₁-C₆alkyl.

In some embodiments, R³ is selected from a group consisting of C₃₋₈cycloalkyl, C₂₋₉heterocycle, C₆₋₁₀aryl, C₁₋₉heteroaryl, and a fused C₅₋₉heteroaryl-cycloalkyl; wherein C₃₋₈cycloalkyl, C₂₋₉heterocycle, C₆₋₁₀aryl, C₁₋₉heteroaryl, and fused C₅₋₉heteroaryl-cycloalkyl are optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R³ is selected from a group consisting of C₂₋₉heterocycle and C₁₋₉heteroaryl; wherein C₂₋₉heterocycle and C₁₋₉heteroaryl are optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R³ is selected from a group consisting of C₂₋₉heterocycle and C₁₋₉heteroaryl; wherein C₂₋₉heterocycle and C₁₋₉heteroaryl are optionally substituted with one or two substituents selected from the group consisting of halo, —CN, C₁₋₆alkyl, —C₁₋₆alkyl-OH, C₁₋₆ haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R³ is selected from a group consisting of pyrazole, imidazole, thiazole, and pyridine; wherein pyrazole, imidazole, thiazole, and pyridine are optionally substituted with one or two substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R³ is selected from a group consisting of pyrazole, imidazole, thiazole, and pyridine; wherein pyrazole, imidazole, thiazole, and pyridine are optionally substituted with one or two substituents selected from the group consisting of halo, C₁₋₆alkyl, and C₃₋₈cycloalkyl.

In some embodiments, R³ is

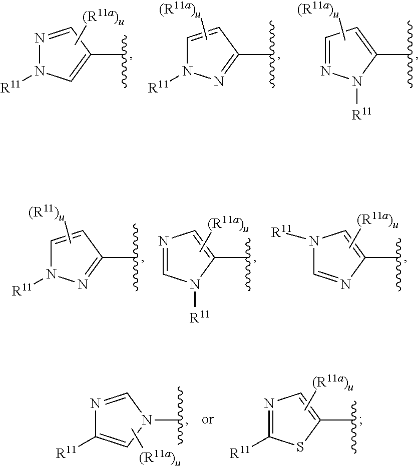

each R¹¹ is C₁-C₆alkyl or C₃-C₆cycloalkyl; R^{11a} is —CN, —OH, C₁-C₆alkyl or C₃-C₆cycloalkyl; and u is 0, 1 or 2.

In some embodiments, R³ is

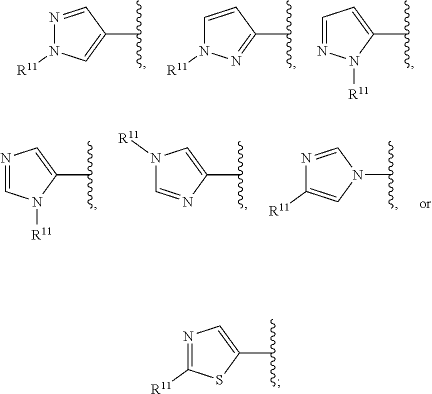

wherein R¹¹ is C₁-C₆alkyl or C₃-C₆cycloalkyl.

In some embodiments, R³ is

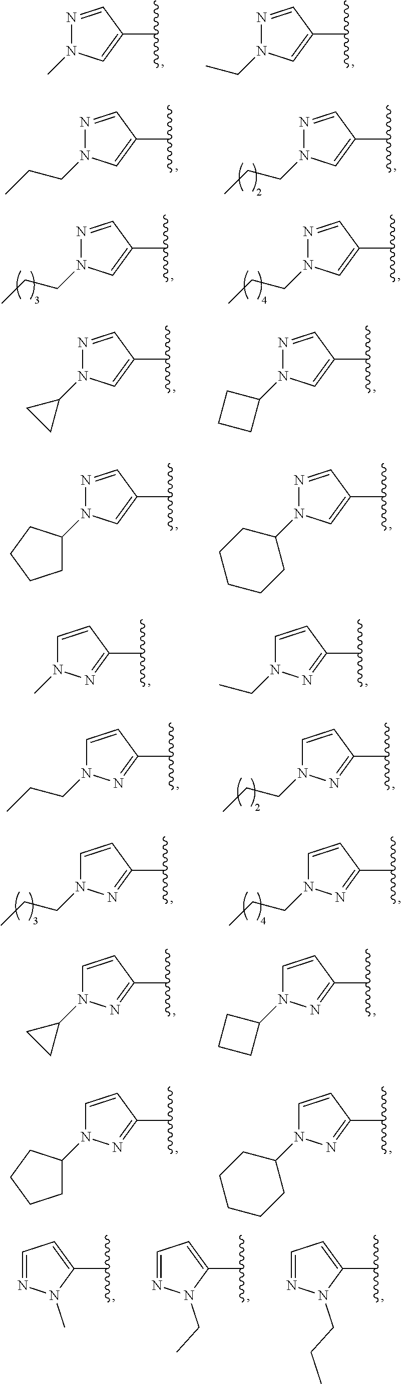

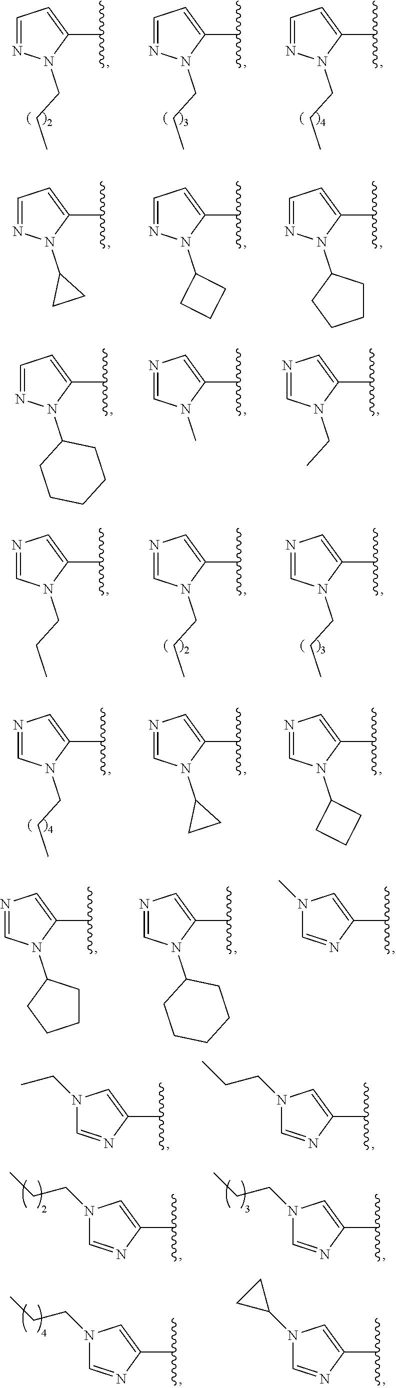

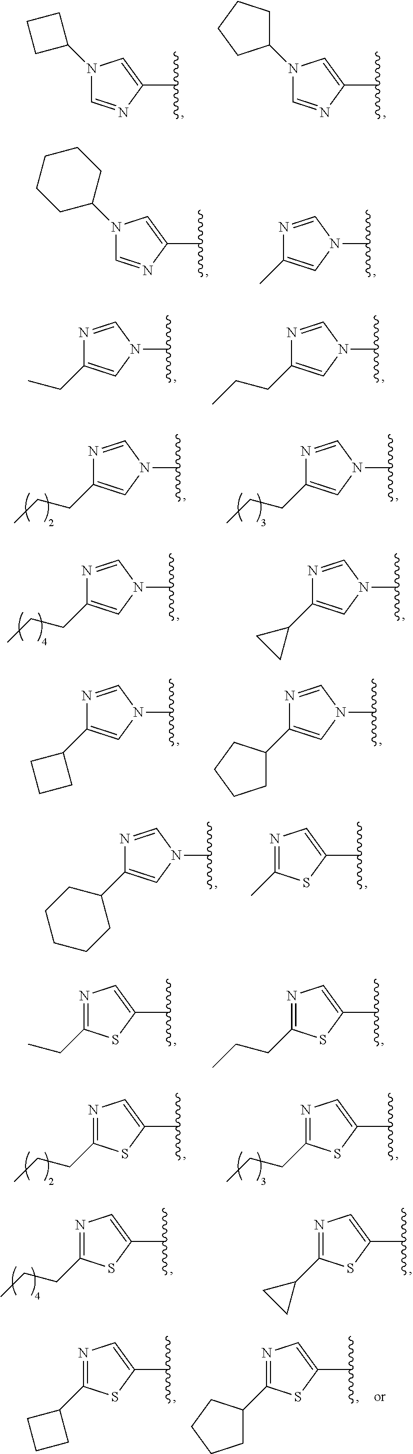

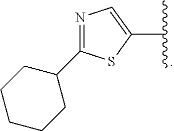 [SMILES: *c1cnc(C2CCCCC2)s1]

In some embodiments, R³ is

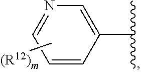

wherein each R¹² is independently halo, C₁-C₆alkyl, or C₃-C₆cycloalkyl; and m is 1 or 2.

In some embodiments, R³ is selected from a group consisting of unsubstituted pyrazole, unsubstituted imidazole, unsubstituted thiazole, and unsubstituted pyridine.

In some embodiments, R³ is —C(═O)N(R⁶)₂ and each R⁶ is independently selected from the group consisting of hydrogen, C₁-C₆alkyl, —C₁-C₆alkyl-O—C₁-C₆alkyl, —C₁-C₆alkyl-C₂₋₉heterocycle, —C₁-C₆alkyl-C₂₋₉heteroaryl, C₃-C₈cycloalkyl, and C₂₋₉heterocycle.

In some embodiments, R³ is

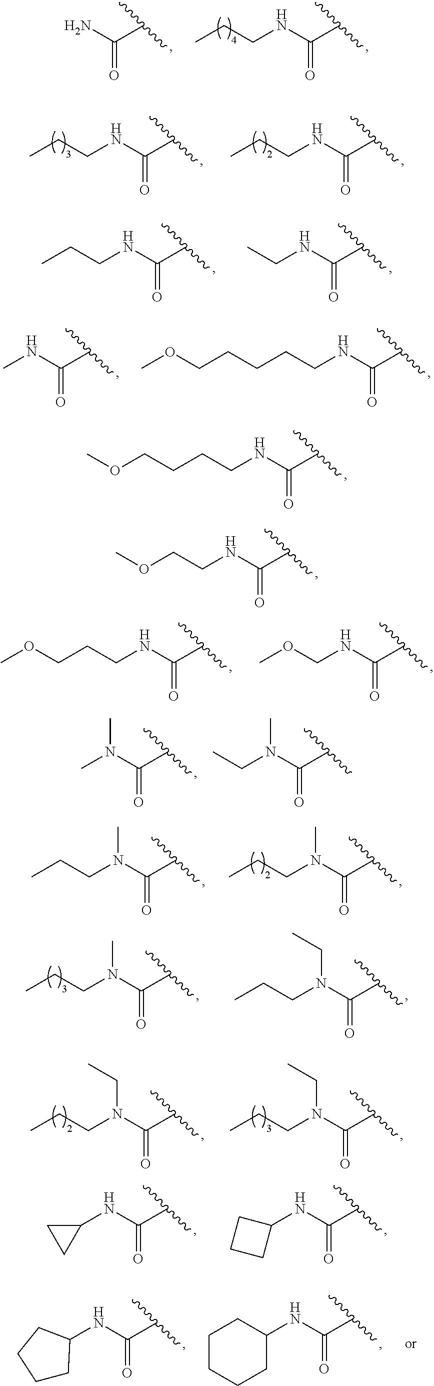

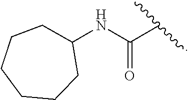 [SMILES: *C(=O)NC1CCCCCC1]

In some embodiments, R³ is

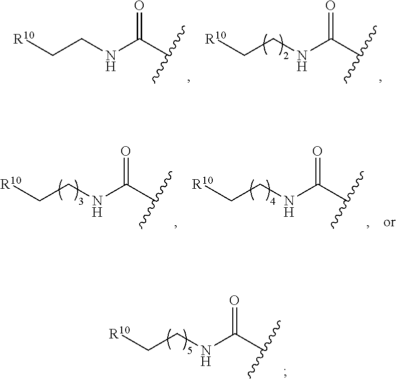

wherein R¹⁰ is a heteroaryl.

In some embodiments, R³ is —OR⁶ and R⁶ is selected from the group consisting of C₁-C₆alkyl, —C₁-C₆alkyl-O—C₁-C₆alkyl, and —C₁-C₆alkyl-C₂₋₉heterocycle. In some embodiments, R² is hydrogen. In some embodiments, R² is C₁-C₆alkyl.

In some embodiments, R¹ is selected from a group consisting of C₃₋₈cycloalkyl, C₂₋₉heterocycle, C₆₋₁₀aryl, C₁₋₉heteroaryl, and a fused C₅₋₉heteroaryl-cycloalkyl; wherein C₁₋₆alkyl, C₂₋₆alkenyl, C₂₋₆alkynyl, C₃₋₈cycloalkyl, C₂₋₉heterocycle, C₆₋₁₀aryl, C₁₋₉heteroaryl, and fused C₅₋₉heteroaryl-cycloalkyl are optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R¹ is selected from a group consisting of a C₁₋₉heteroaryl and a fused C₅₋₉heteroaryl-cycloalkyl; wherein the C₁₋₉heteroaryl and fused C₅₋₉heteroaryl-cycloalkyl are optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R¹ is selected from a group consisting of a C₁₋₉heteroaryl and a fused C₅₋₉heteroaryl-cycloalkyl; wherein the C₁₋₉heteroaryl and fused C₅₋₉heteroaryl-cycloalkyl are optionally substituted with one or two substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R¹ is selected from a group consisting of triazole, imidazole, oxazole, isoxazole, oxadiazole, and tetrazole; wherein triazole, imidazole, oxazole, isoxazole, oxadiazole, and tetrazole are optionally substituted with one or two substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R¹ is selected from a group consisting of triazole, imidazole, oxazole, isoxazole, oxadiazole, and tetrazole; wherein triazole, imidazole, oxazole, isoxazole, oxadiazole, and tetrazole are optionally substituted with one or two substituents selected from the group consisting of halo, C₁₋₆alkyl, and C₃₋₈cycloalkyl.

In some embodiments, R¹ is

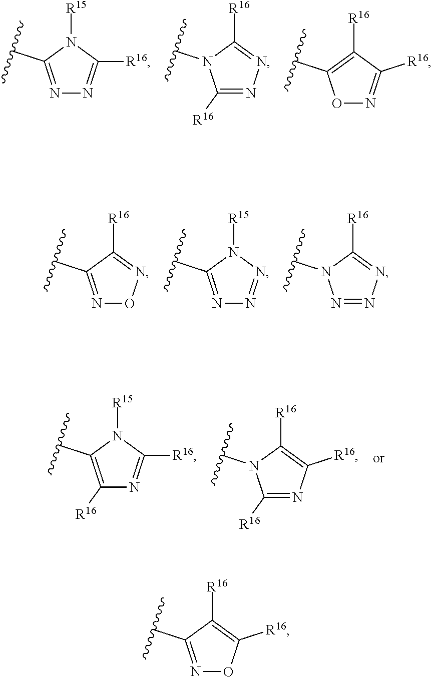

wherein R¹⁵ is C₁₋₆alkyl or C₃₋₈cycloalkyl; and R¹⁶ is hydrogen, halo, —CN, C₁₋₆alkyl or C₃₋₈cycloalkyl.

In some embodiments, R¹ is

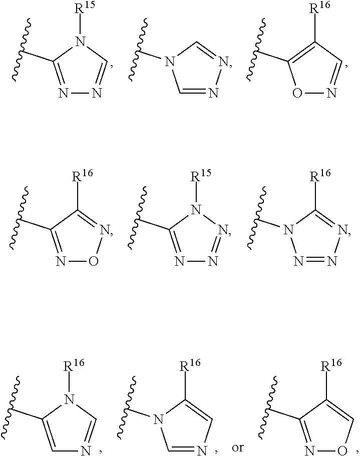

wherein R¹⁵ is C₁₋₆alkyl or C₃₋₈cycloalkyl; and R¹⁶ is halo, —CN, C₁₋₆alkyl or C₃₋₈cycloalkyl.

In some embodiments, R¹ is

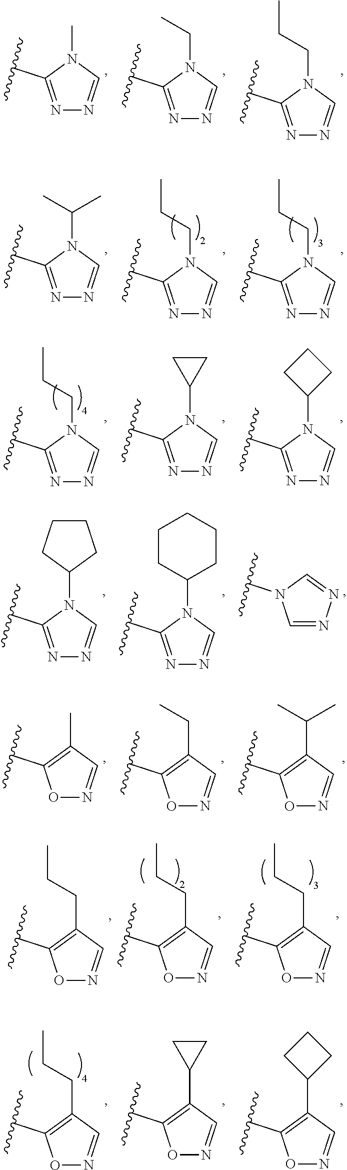

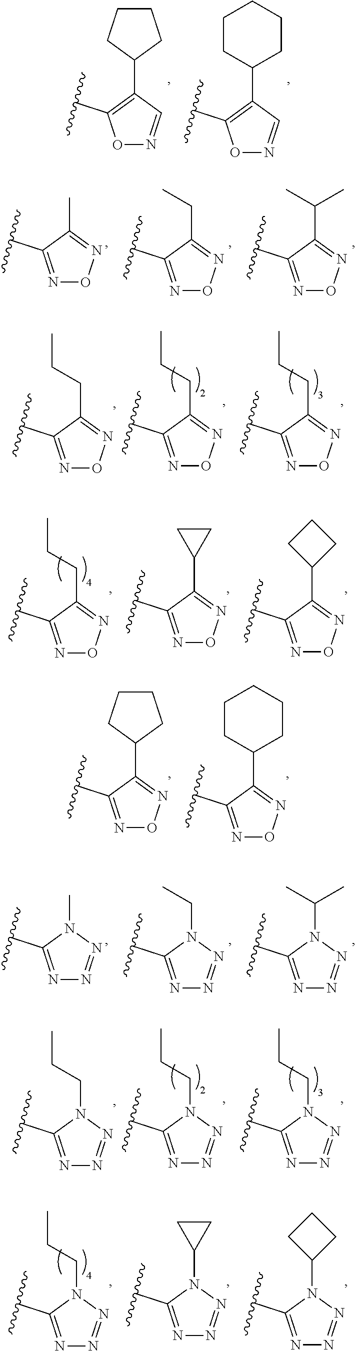

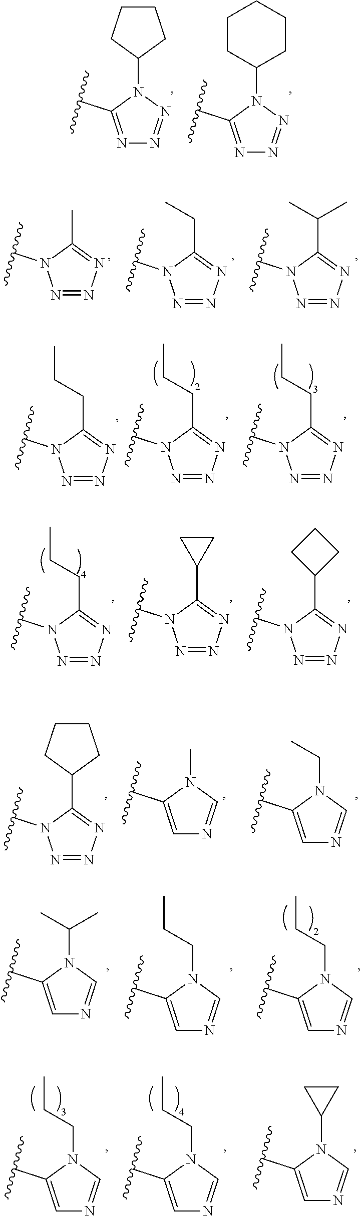

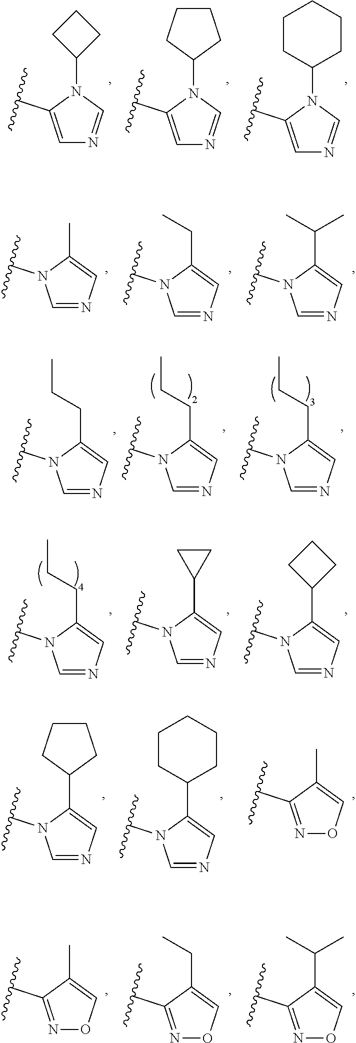

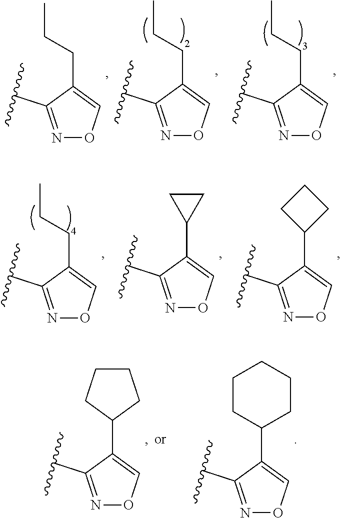

In some embodiments, R¹ is

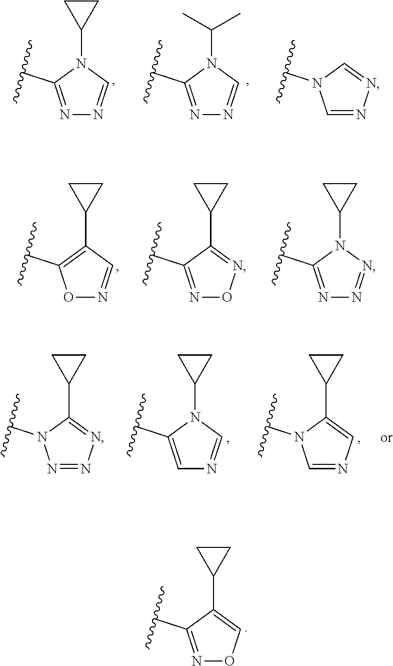

In some embodiments, R¹ is

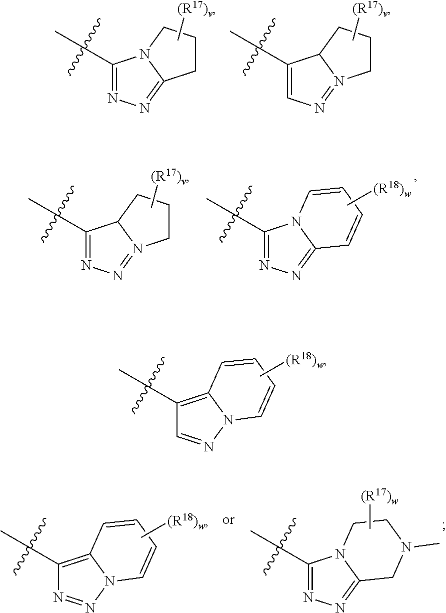

wherein each R¹⁷ is independently hydrogen, halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³; each R¹⁸ is independently hydrogen, halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³; v is 0, 1, 2, 3, 4, 5, or 6; and w is 0, 1, 2, 3, or 4. In some embodiments, each R¹⁷ is independently hydrogen, halo, —CN, C₁₋₆alkyl, —C₁₋₆alkyl-OH, C₁₋₆haloalkyl, or C₃₋₈cycloalkyl; each R¹⁸ is independently hydrogen, halo, —CN, C₁₋₆alkyl, —C₁₋₆alkyl-OH, C₁₋₆haloalkyl, or C₃₋₈cycloalkyl.

In some embodiments, each R¹⁷ is independently hydrogen, halo, —CN, C₁₋₆alkyl, —C₁₋₆ alkyl-OH, or C₁₋₆haloalkyl; each R¹⁸ is independently hydrogen, halo, —CN, C₁₋₆alkyl, —C₁₋₆alkyl-OH, or C₁₋₆haloalkyl.

In some embodiments, each R¹⁷ is independently hydrogen, halo, —CN, or C₁₋₆alkyl; each R¹⁸ is independently is hydrogen, halo, —CN, or C₁₋₆alkyl.

In some embodiments, R¹ is

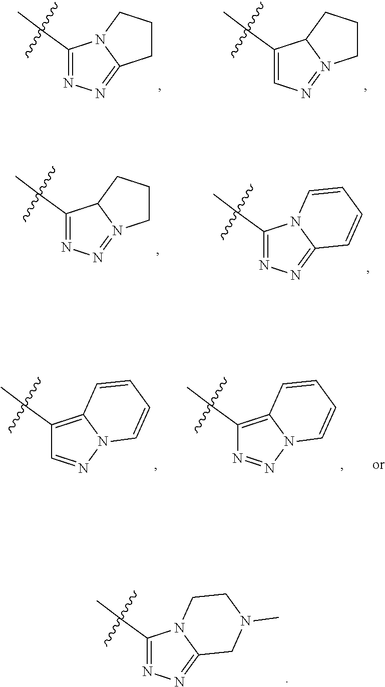

In some embodiments described herein is a method for treating an organ disease or disorder in a subject in need thereof comprising administering to the subject a therapeutically effective amount of a compound of Formula II, or a pharmaceutically acceptable salt or solvate thereof:

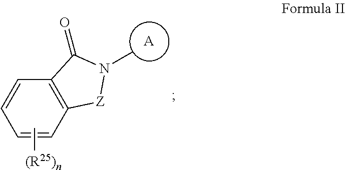

wherein

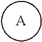

is

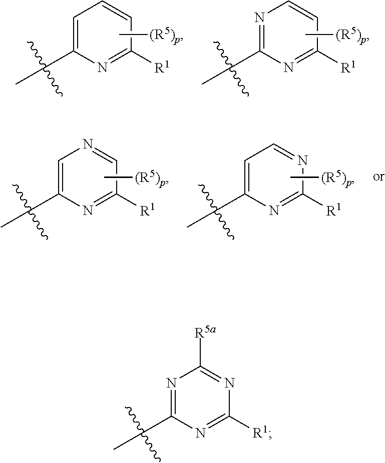

R¹ is

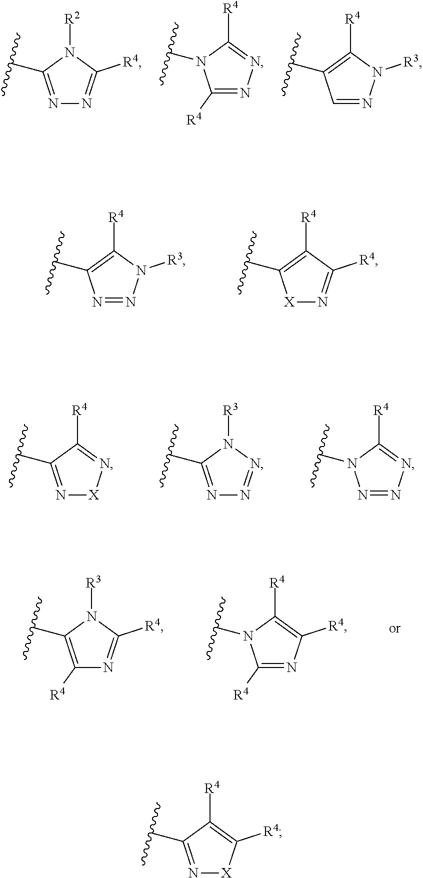

Z is O, S, C(═O), N(R⁸), or C(R⁹)₂;
X is O or S;
R² is C₃₋₆cycloalkyl;
R³ is selected from a group consisting of hydrogen, C₁₋₆alkyl, and C₃₋₆cycloalkyl;
each R⁴ is independently selected from a group consisting of hydrogen, halo, C₁₋₆alkyl, and C₃₋₆cycloalkyl;
   or one R⁴ and another R², R³, or R⁴, together with the atoms to which they are attached, form a 5- or 6-membered ring that is optionally containing one or two heteroatoms selected from O, N, and S; wherein the 5- or 6-membered ring is saturated, unsaturated, or aromatic; and wherein the 5- or 6-membered ring is optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³;
each R⁵ is independently selected from a group consisting of halogen and C₁₋₆alkyl;
R^{5a} is selected from the group consisting of hydrogen and C₁-C₆alkyl;
R²⁵ is selected from a group consisting of halogen, —CN, —OH, —OR⁶, —SR⁶, —S(═O)R⁷, —NO₂, —N(R⁶)₂, —S(═O)₂R⁷, —NHS(═O)₂R⁷, —S(═O)₂N(R⁶)₂, —C(═O)R⁷, —C(═O)OR⁶, —OC(═O)R⁷, —C(═O)N(R⁶)₂, —OC(═O)N(R⁶)₂, —NR⁶C(═O)N(R⁶)₂, —NR⁶C(═O)R⁷, —NR⁶C(═O)OR⁶, C₁₋₆alkyl, C₂₋₆alkenyl, C₂₋₆alkynyl, C₃₋₈cycloalkyl, C₂₋₉heterocycle, C₆₋₁₀aryl, C₁₋₉heteroaryl, and a fused C₅₋₉heteroaryl-cycloalkyl; wherein C₁₋₆alkyl, C₂₋₆alkenyl, C₂₋₆alkynyl, C₃₋₈cycloalkyl, C₂₋₉heterocycle, C₆₋₁₀aryl, C₁₋₉heteroaryl, and fused C₅₋₉heteroaryl-cycloalkyl are optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³;
each R⁶ is independently selected from the group consisting of hydrogen, C₁-C₆alkyl, —C₁-C₆alkyl-O—C₁-C₆alkyl, —C₁-C₆alkyl-C₂₋₉heterocycle, —C₁-C₆alkyl-C₂₋₉heteroaryl, C₃-C₈cycloalkyl, and C₂₋₉heterocycle, or two R⁶ on the same heteroatom are taken together with that heteroatom to which they are attached to form a C₂₋₉heterocycle or a C₂₋₉heteroaryl;
each R⁷ is independently selected from the group consisting of C₁-C₆alkyl and C₃-C₈cycloalkyl;
R⁸ is selected from the group consisting of hydrogen and C₁-C₆alkyl;
each R⁹ is independently selected from the group consisting of hydrogen, halogen, and C₁-C₆alkyl;
each R¹³ is independently selected from the group consisting of hydrogen, C₁-C₆alkyl, and C₃-C₈cycloalkyl; or two R¹³ on the same heteroatom are taken together with that heteroatom to which they are attached to form a C₂₋₉heterocycle;
each R¹⁴ is independently selected from the group consisting of C₁-C₆alkyl and C₃-C₈cycloalkyl;
n is 0, 1, 2, 3 or 4;
p is 0, 1, 2, or 3; and
q is 0, 1, or 2.

In some embodiments, R²⁵ is selected from a group consisting of C₃₋₈cycloalkyl, C₂₋₉heterocycle, C₆₋₁₀aryl, C₁₋₉heteroaryl, and a fused C₅₋₉heteroaryl-cycloalkyl; wherein C₃₋₈cycloalkyl, C₂₋₉heterocycle, C₆₋₁₀aryl, C₁₋₉heteroaryl, and fused C₅₋₉heteroaryl-cycloalkyl, wherein C₃₋₈cycloalkyl, C₂₋₉heterocycle, C₆₋₁₀aryl, C₁₋₉heteroaryl, and fused C₅₋₉heteroaryl-cycloalkyl are optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R²⁵ is selected from a group consisting of C₂₋₉heterocycle and C₁₋₉heteroaryl; wherein C₂₋₉heterocycle and C₁₋₉heteroaryl are optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R²⁵ is selected from a group consisting of C₂₋₉heterocycle and C₁₋₉heteroaryl; wherein C₂₋₉heterocycle and C₁₋₉heteroaryl are optionally substituted with one or two substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R²⁵ is selected from a group consisting of pyrazole, imidazole, thiazole, and pyridine; wherein pyrazole, imidazole, thiazole, and pyridine are optionally substituted with one or two substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R²⁵ is selected from a group consisting of pyrazole, imidazole, thiazole, and pyridine; wherein pyrazole, imidazole, thiazole, and pyridine are optionally substituted with one or two substituents selected from the group consisting of halo, C₁₋₆alkyl, and C₃₋₈cycloalkyl.

In some embodiments, R²⁵ is

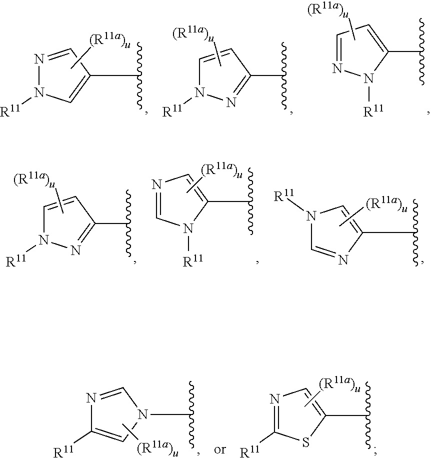

each R¹¹ is C₁-C₆alkyl or C₃-C₆cycloalkyl; R^{11a} is —CN, —OH, C₁-C₆alkyl or C₃-C₆cycloalkyl; and u is 0, 1 or 2.

In some embodiments, R²⁵ is

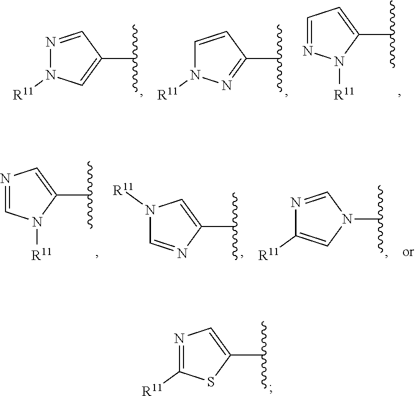

wherein R¹¹ is C₁-C₆alkyl or C₃-C₆cycloalkyl.

In some embodiments, R²⁵ is

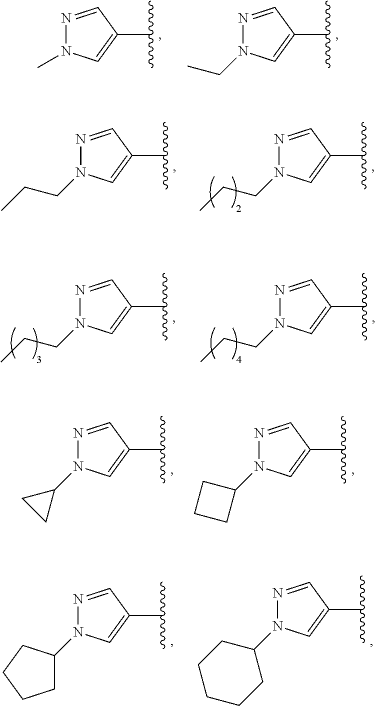

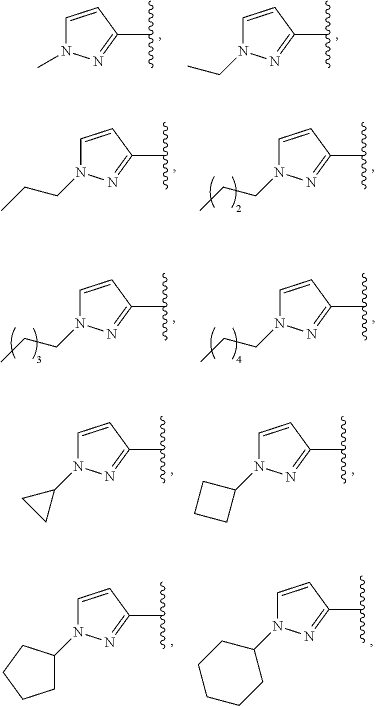

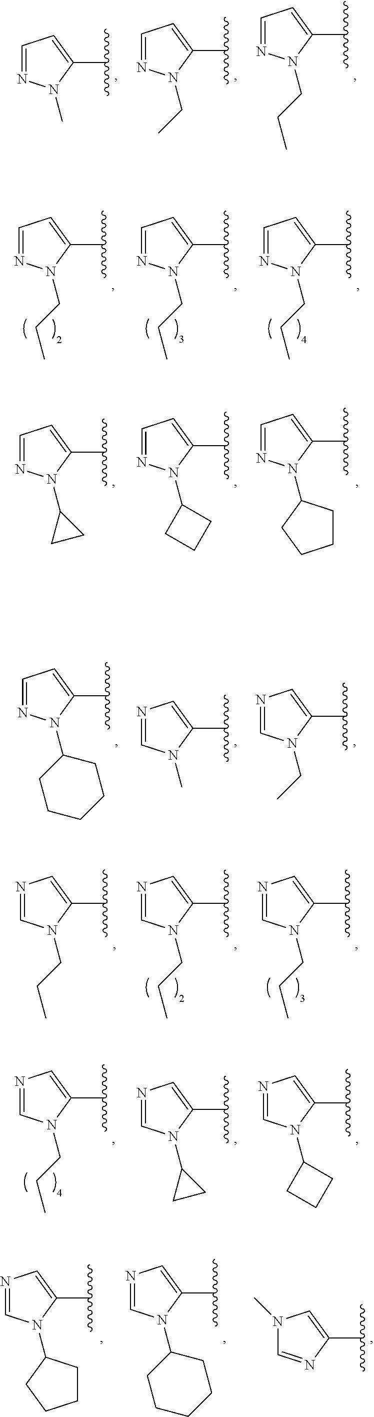

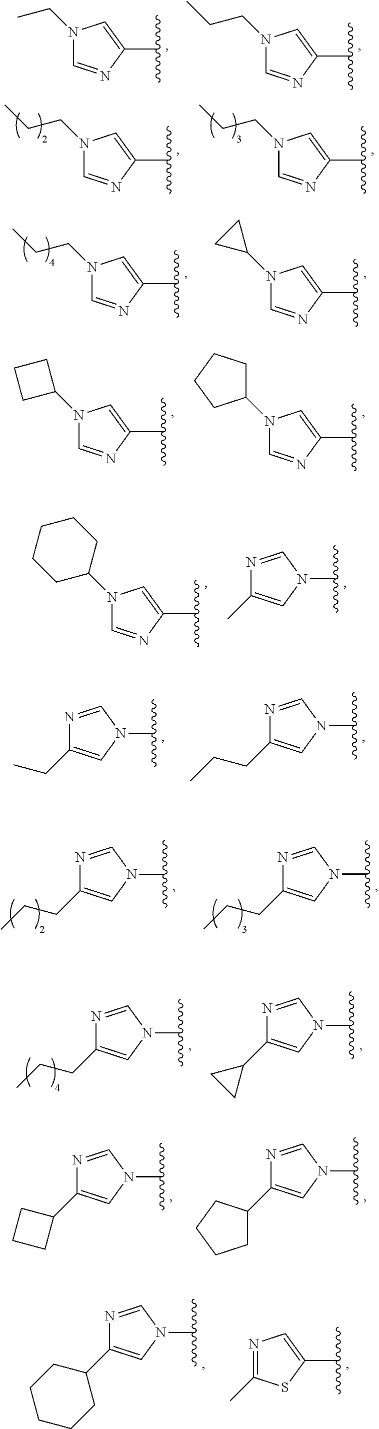

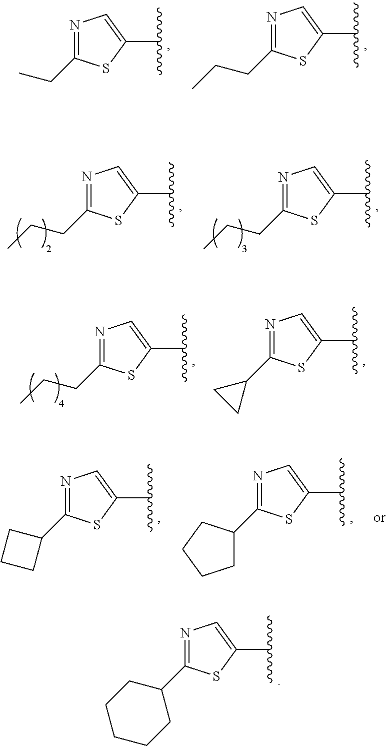

In some embodiments, R²⁵ is

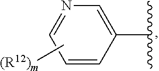

wherein R¹² is halo, C₁-C₆alkyl, or C₃-C₆cycloalkyl; and
m is 1 or 2.

In some embodiments, R²⁵ is selected from a group consisting of unsubstituted pyrazole, unsubstituted imidazole, unsubstituted thiazole, and unsubstituted pyridine.

In some embodiments, R²⁵ is —C(═O)N(R⁶)₂ and each R⁶ is independently selected from the group consisting of hydrogen, C₁-C₆alkyl, —C₁-C₆alkyl-O—C₁-C₆alkyl, —C₁-C₆alkyl-C₂₋₉heterocycle, —C₁-C₆alkyl-C₂₋₉heteroaryl, C₃-C₈cycloalkyl, and C₂₋₉heterocycle.

In some embodiments, R²⁵ is

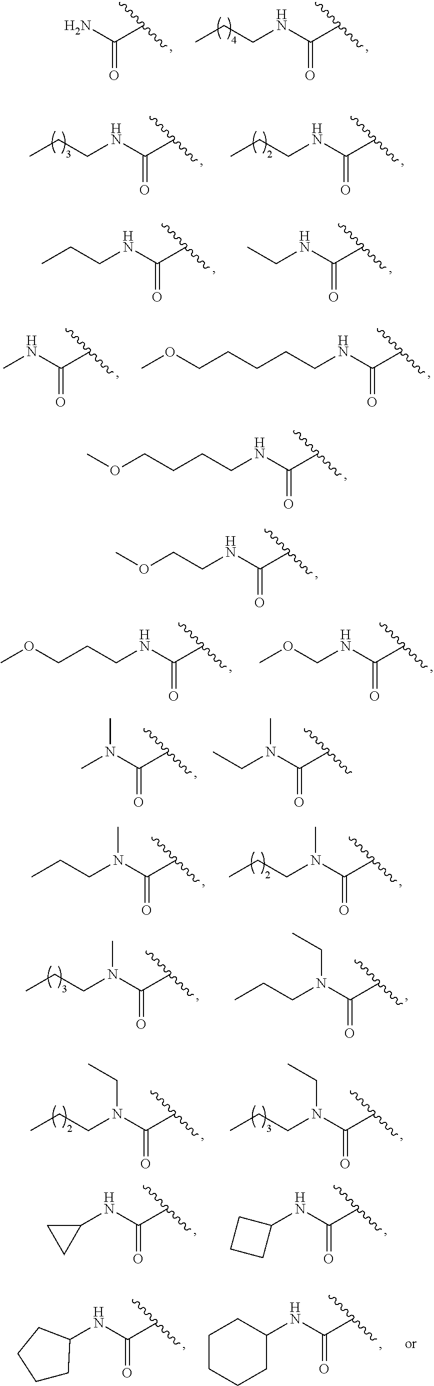

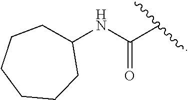 [SMILES: *C(=O)NC1CCCCCC1]

In some embodiments, R²⁵ is

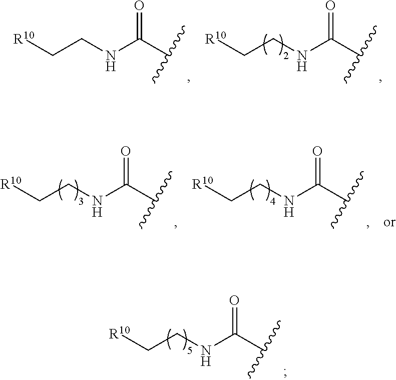

wherein R¹⁰ is a heteroaryl.

In some embodiments, R²⁵ is —C(═O)N(R⁶)₂ and two R⁶ are taken together with that heteroatom to which they are attached to form a C₂₋₉heterocycle or a C₂₋₉heteroaryl.

In some embodiments, R²⁵ is

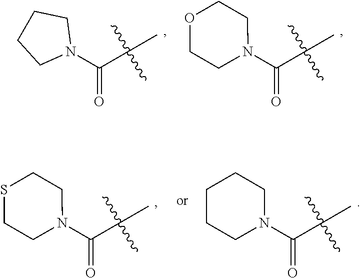

In some embodiments, R²⁵ is —OR⁶ and R⁶ is selected from the group consisting of C₁-C₆alkyl, —C₁-C₆alkyl-O—C₁-C₆alkyl, and —C₁-C₆alkyl-C₂₋₉heterocycle.

In some embodiments,

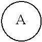

is

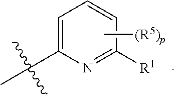

In some embodiments, p is 0.

In some embodiments,

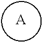

is

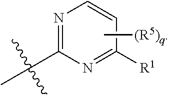

In some embodiments,

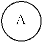

is

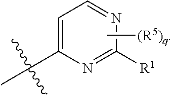

In some embodiments, q is 0.

In some embodiments, n is 0. In some embodiments, n is 1. In some embodiments, Z is C(R⁹)₂. In some embodiments, R⁹ is H.

In some embodiments, R¹ is

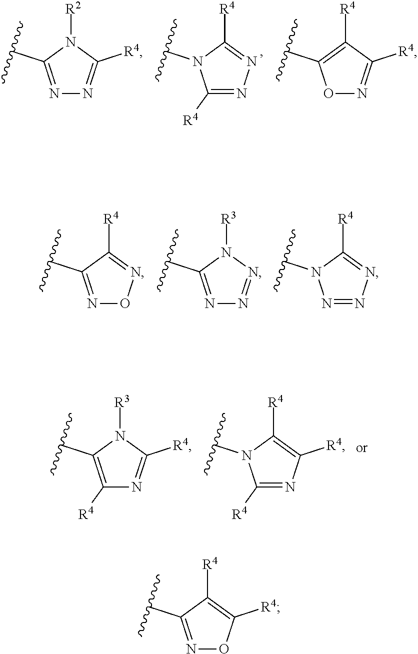

wherein R² is C₃₋₆cycloalkyl; R³ is selected from a group consisting of hydrogen C₁₋₆alkyl and C₃₋₆ cycloalkyl; each R⁴ is independently selected from a group consisting of hydrogen, halogen, C₁₋₆ alkyl, and C₃₋₆cycloalkyl; or one R⁴ and another R², R³, or R⁴, together with the atoms to which they are attached, form a 5- or 6-membered ring that is optionally containing one or two heteroatoms selected from O, N, and S; wherein the 5- or 6-membered ring is saturated, unsaturated or aromatic; and wherein the 5- or 6-membered ring is optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R¹ is

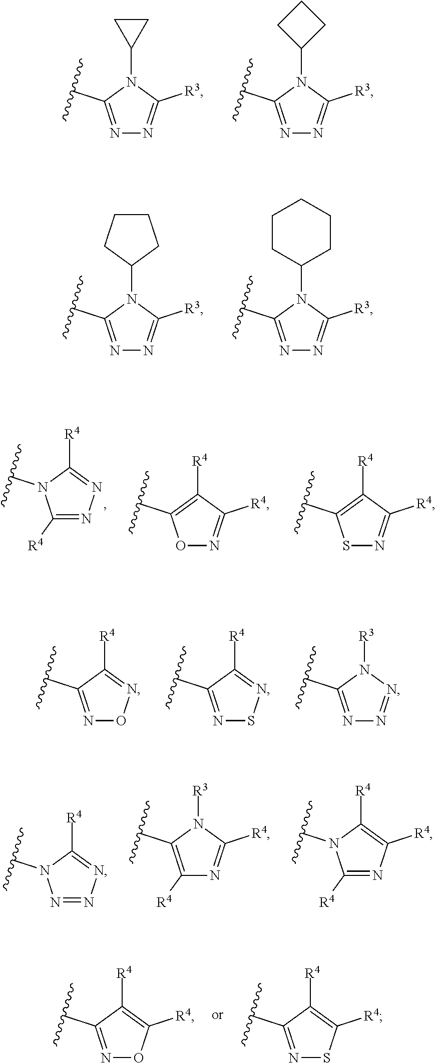

wherein R³ is selected from a group consisting of C₁₋₆alkyl and C₃₋₆cycloalkyl; and
each R⁴ is independently selected from a group consisting of hydrogen, halogen, C₁₋₆alkyl, and C₃₋₆cycloalkyl.

In some embodiments, R¹ is

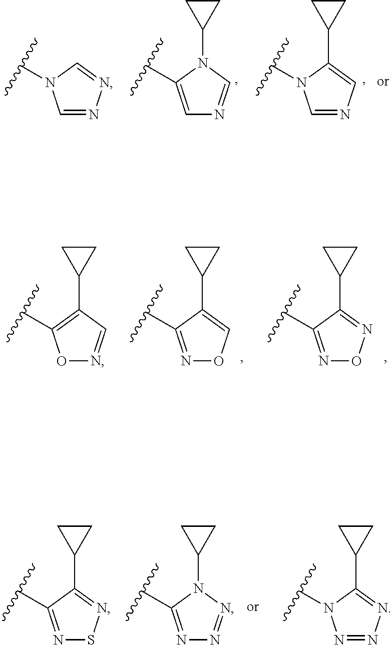

In some embodiments, R¹ is

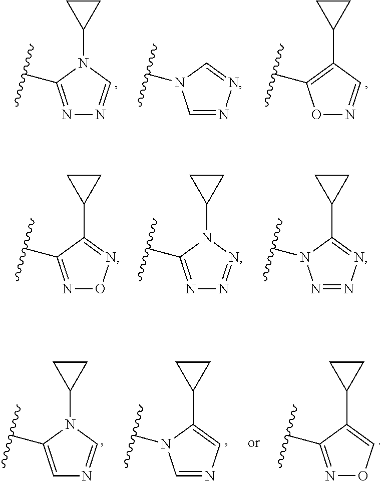

In some embodiments, R¹ is

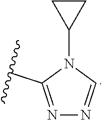 [SMILES: *c1nncn1C1CC1]

In some embodiments, R¹ is

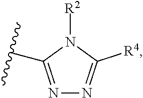

wherein R² and R⁴, together with the atoms to which they are attached, form a 5- or 6-membered ring that is optionally containing one or two heteroatoms selected from O, N, and S; wherein the 5- or 6-membered ring is saturated, unsaturated or aromatic; and wherein the 5- or 6-membered ring is optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R¹ is

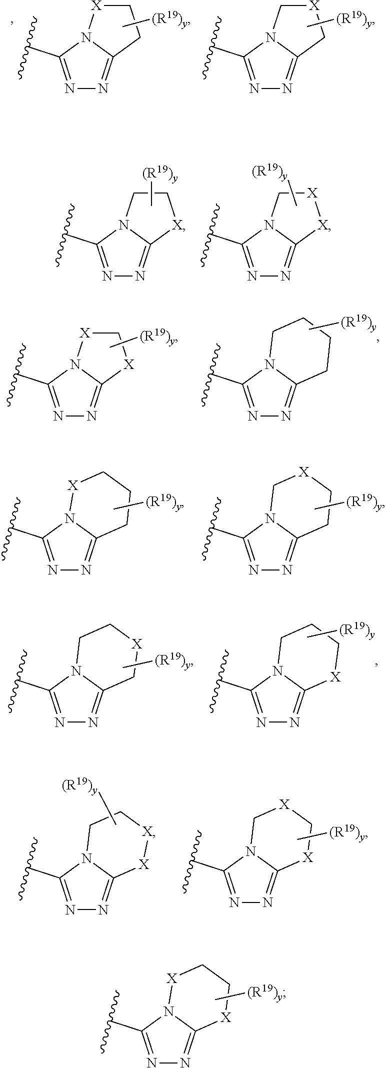

wherein X is 0, N or S; each R¹⁹ is independently halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³; and y is 0, 1, 2, 3 or 4.

In some embodiments, R¹ is

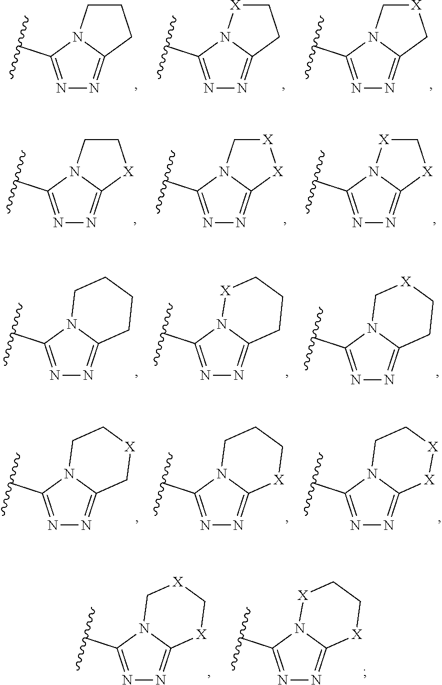

wherein X is 0, N or S.

In some embodiments, R¹ is

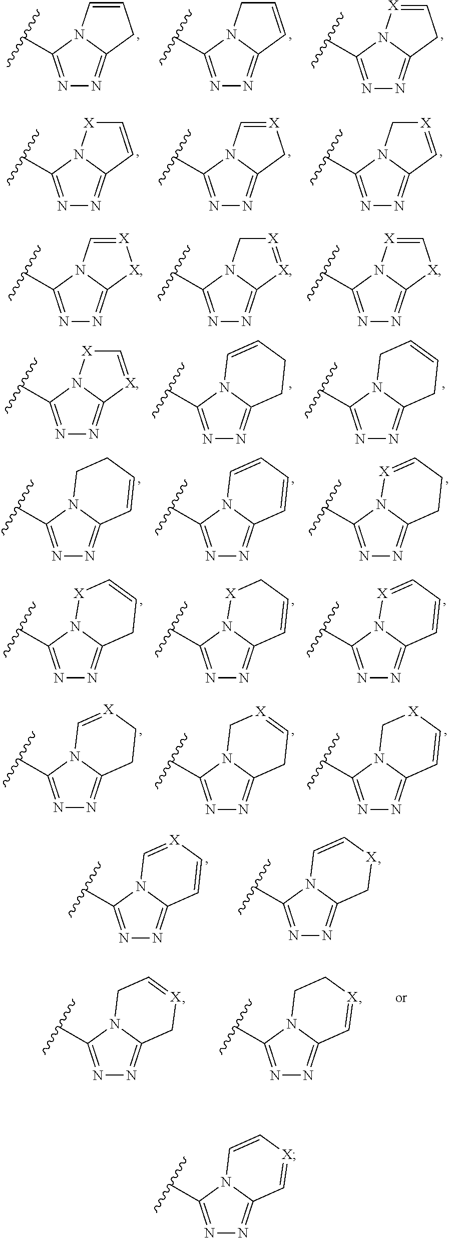

wherein X is N.

In some embodiments, R¹ is

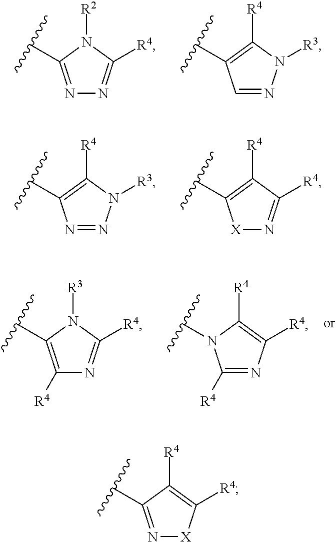

wherein X is 0 or S; and
one R⁴ and another R², R³, or R⁴, together with the atoms to which they are attached, form a 5- or 6-membered ring that is optionally containing one or two heteroatoms selected from O, N, and S; wherein the 5- or 6-membered ring is saturated, unsaturated or aromatic; and wherein the 5- or 6-membered ring is optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R¹ is

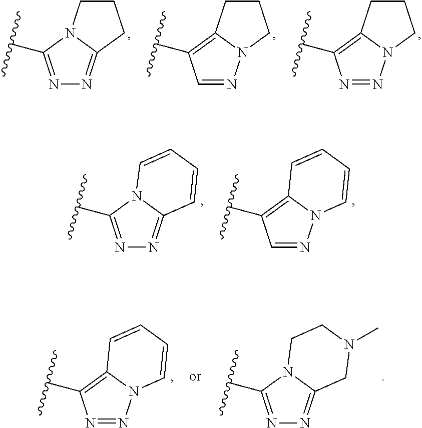

In some embodiments, R¹ is

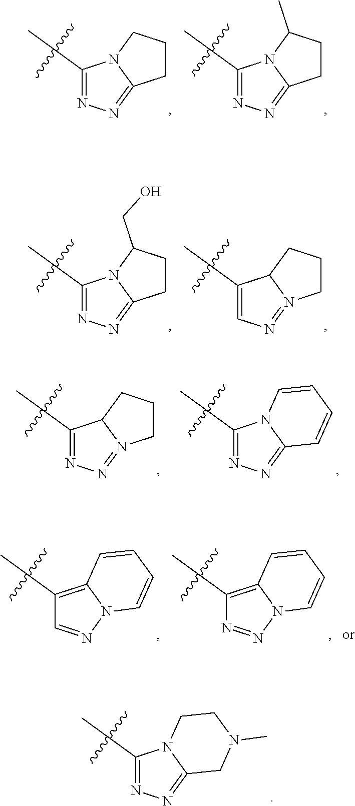

In some embodiments described herein is a method for treating an organ disease or disorder in a subject in need thereof comprising administering to the subject a therapeutically effective amount of a compound of Formula III, or a pharmaceutically acceptable salt or solvate thereof:

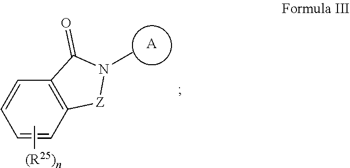

wherein

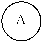

is

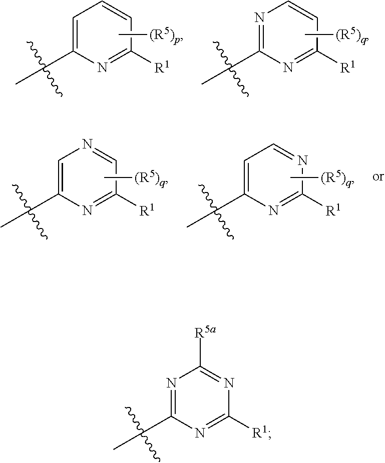

R¹ is

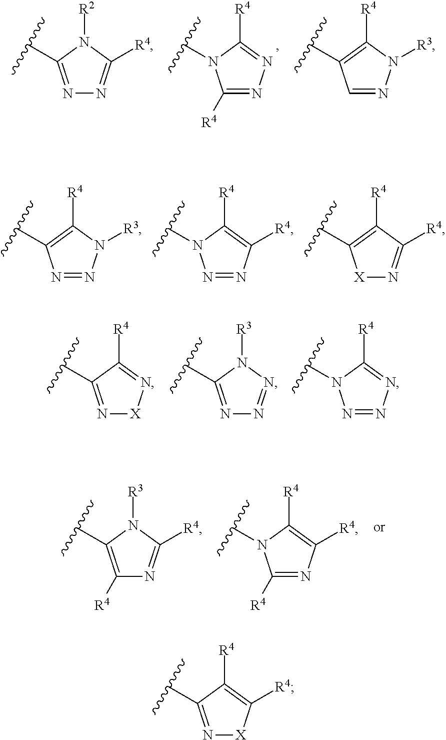

Z is 0, S, C(═O), N(R⁸), or C(R⁹)₂;
X is O or S;
R² is C₃₋₆cycloalkyl;
R³ is selected from a group consisting of hydrogen, C₁₋₆alkyl, and C₃₋₆cycloalkyl;
each R⁴ is independently selected from a group consisting of hydrogen, halo, C₁₋₆alkyl, and C₃₋₆cycloalkyl;
   or one R⁴ and another R², R³, or R⁴, together with the atoms to which they are attached, form a 5- or 6-membered ring that is optionally containing one or two heteroatoms selected from O, N, and S; wherein the 5- or 6-membered ring is saturated, unsaturated, or aromatic; and wherein the 5- or 6-membered ring is optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³;
each R⁵ is independently selected from a group consisting of halogen and C₁₋₆alkyl;
R^{5a} is selected from the group consisting of hydrogen and C₁-C₆alkyl;
each R²⁵ is independently selected from a group consisting of halogen, —CN, —OH, —OR⁶, —SR⁶, —S(═O)R⁷, —NO₂, —N(R⁶)₂, —S(═O)₂R⁷, —NHS(═O)₂R⁷, —S(═O)₂N(R⁶)₂, —C(═O)R⁷, —C(═O)OR⁶, —OC(═O)R⁷, —C(═O)N(R⁶)₂, —OC(═O)N(R⁶)₂, —NR⁶C(═O)N(R⁶)₂, —NR⁶C(═O)R⁷, —NR⁶C(═O)OR⁶, C₁₋₆alkyl, C₂₋₆alkenyl, C₂₋₆alkynyl, C₃₋₈cycloalkyl, C₂₋₉heterocycle, C₆₋₁₀aryl, C₁₋₉heteroaryl, and a fused C₅₋₉heteroaryl-cycloalkyl; wherein C₁₋₆alkyl, C₂₋₆alkenyl, C₂₋₆alkynyl, C₃₋₈cycloalkyl, C₂₋₉heterocycle, C₆₋₁₀aryl, C₁₋₉heteroaryl, and fused C₅₋₉heteroaryl-cycloalkyl are optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.
each R⁶ is independently selected from the group consisting of hydrogen, C₁-C₆alkyl, —C₁-C₆alkyl-O—C₁-C₆alkyl, —C₁-C₆alkyl-C₂₋₉heterocycle, —C₁-C₆alkyl-C₂₋₉heteroaryl, C₃-C₈cycloalkyl, —C₃-C₈cycloalkyl-phenyl, and C₂₋₉heterocycle, wherein C₁-C₆alkyl, —C₁-C₆alkyl-O—C₁-C₆alkyl, —C₁-C₆alkyl-C₂₋₉heterocycle, —C₁-C₆alkyl-C₂₋₉heteroaryl, C₃-C₈cycloalkyl, —C₃-C₈cycloalkyl-phenyl, and C₂₋₉heterocycle are optionally substituted with one, two, or three substituents selected from the group consisting of halo, —OR⁸, —SR⁸, —N(R⁸)₂, —C₁₋₆alkyl, —O—C₁₋₆alkyl, —C(═O)R¹⁴, —C(═O)OR¹³, and —N(R¹³)C(═O)R¹⁴; or two R⁶ on the same heteroatom are taken together with that heteroatom to which they are attached to form a C₂₋₉heterocycle or a C₂₋₉heteroaryl, wherein C₂₋₉heterocycle or C₂₋₉heteroaryl are optionally substituted with one, two, or three substituents selected from the group consisting of halo, —OR⁸, —SR⁸, —N(R⁸)₂, —C₁₋₆alkyl, —O—C₁₋₆alkyl, —C(═O)R¹⁴, —C(═O)OR¹³, and —N(R¹³)C(═O)R¹⁴;
each R⁷ is independently selected from the group consisting of C₁-C₆alkyl, C₃-C₈cycloalkyl, and C₂₋₉heterocycle, wherein C₃-C₈cycloalkyl and C₂₋₉heterocycle are optionally substituted with one, two, or three substituents selected from the group consisting of halo, oxo, —OR⁸, —SR⁸, —N(R⁸)₂, —C₁₋₆alkyl, —O—C₁₋₆alkyl, —C(═O)R¹⁴, —C(═O)OR¹³, and —N(R¹³)C(═O)R¹⁴;
each R⁸ is independently selected from the group consisting of hydrogen and C₁-C₆alkyl;
each R⁹ is independently selected from the group consisting of hydrogen, halogen, and C₁-C₆alkyl; each R¹³ is independently selected from the group consisting of hydrogen, C₁-C₆alkyl, and C₃-C₈cycloalkyl; or two R¹³ on the same heteroatom are taken together with that heteroatom to which they are attached to form a C₂₋₉heterocycle;
each R¹⁴ is independently selected from the group consisting of C₁-C₆alkyl and C₃-C₈cycloalkyl;
n is 0, 1, 2, 3, or 4;
p is 0, 1, 2, or 3; and
q is 0, 1, or 2.

In some embodiments, R²⁵ is selected from a group consisting of C₃₋₈cycloalkyl, C₂₋₉heterocycle, C₆₋₁₀aryl, C₁₋₉heteroaryl, and a fused C₅₋₉heteroaryl-cycloalkyl; wherein C₃₋₈cycloalkyl, C₂₋₉heterocycle, C₆₋₁₀aryl, C₁₋₉heteroaryl, and fused C₅₋₉heteroaryl-cycloalkyl, wherein C₃₋₈cycloalkyl, C₂₋₉heterocycle, C₆₋₁₀aryl, C₁₋₉heteroaryl, and fused C₅₋₉heteroaryl-cycloalkyl are optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R²⁵ is selected from a group consisting of C₂₋₉heterocycle and C₁₋₉heteroaryl; wherein C₂₋₉heterocycle and C₁₋₉heteroaryl are optionally substituted with one, two, or three substituents selected from the group consisting of halo, —CN, C₁₋₆alkyl, —C₁₋₆alkyl-OH, C₁₋₆ haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R²⁵ is selected from a group consisting of C₂₋₉heterocycle and C₁₋₉heteroaryl; wherein C₂₋₉heterocycle and C₁₋₉heteroaryl are optionally substituted with one or two substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R²⁵ is selected from a group consisting of pyrazole, imidazole, thiazole, and pyridine; wherein pyrazole, imidazole, thiazole, and pyridine are optionally substituted with one or two substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R²⁵ is selected from a group consisting of pyrazole, imidazole, thiazole, and pyridine; wherein pyrazole, imidazole, thiazole, and pyridine are optionally substituted with one or two substituents selected from the group consisting of halo, C₁₋₆alkyl, and C₃₋₈cycloalkyl.

In some embodiments, R²⁵ is

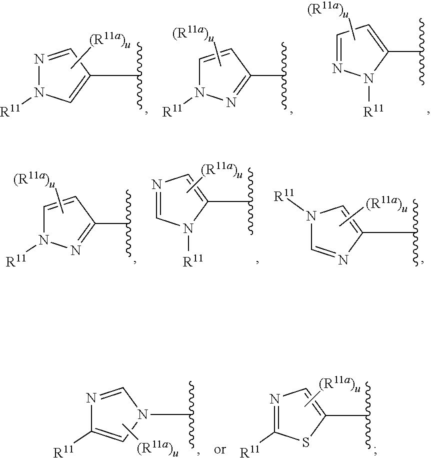

each R¹¹ is C₁-C₆alkyl or C₃-C₆cycloalkyl; R¹¹ is —CN, —OH, C₁-C₆alkyl or C₃-C₆cycloalkyl; and u is 0, 1 or 2.

In some embodiments, R²⁵ is

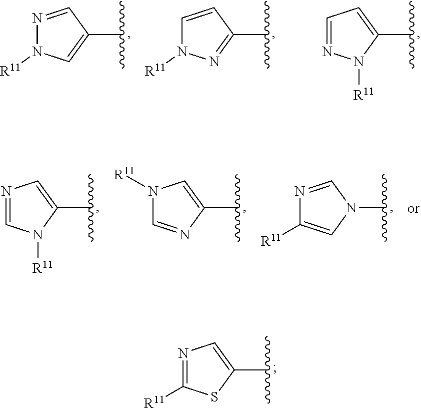

wherein R¹¹ is C₁-C₆alkyl or C₃-C₆cycloalkyl.

In some embodiments, R²⁵ is

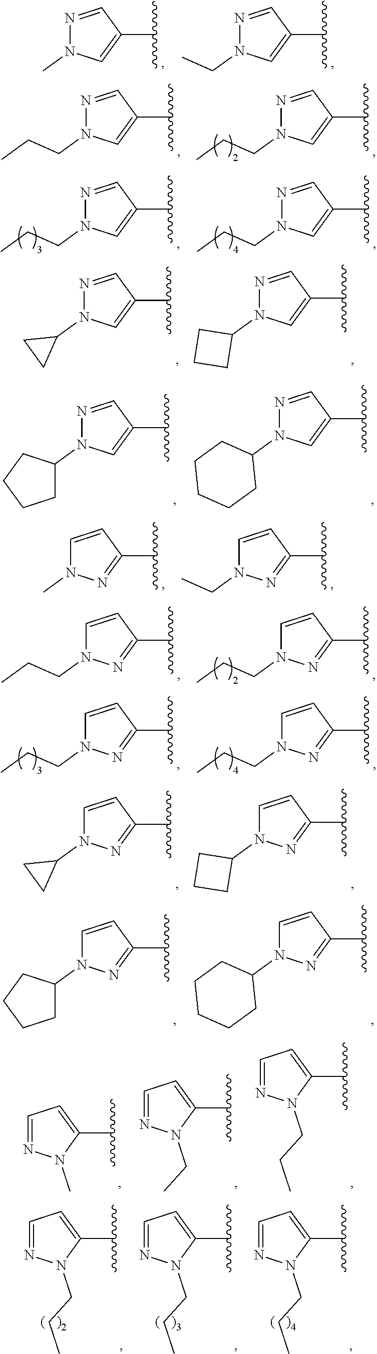

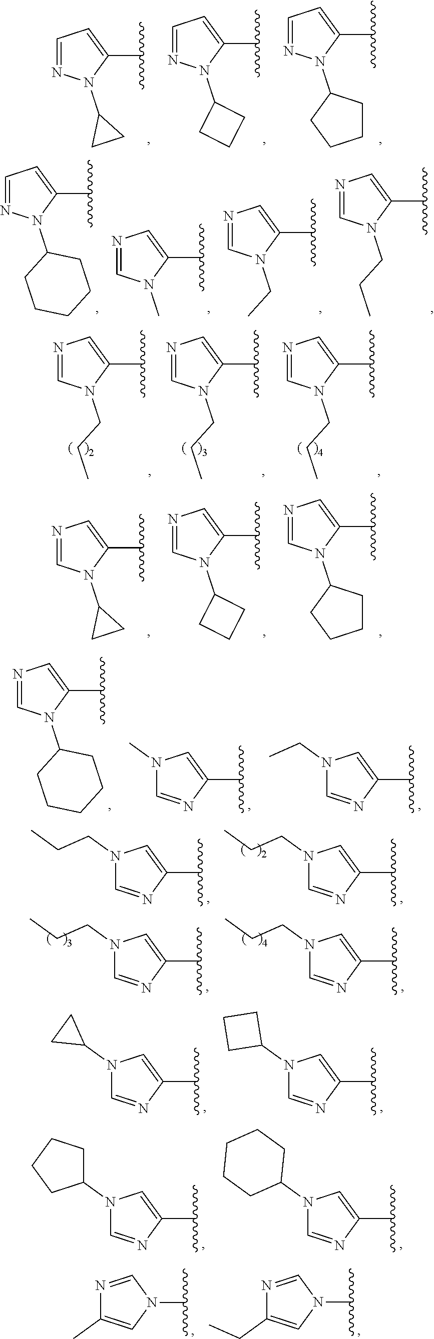

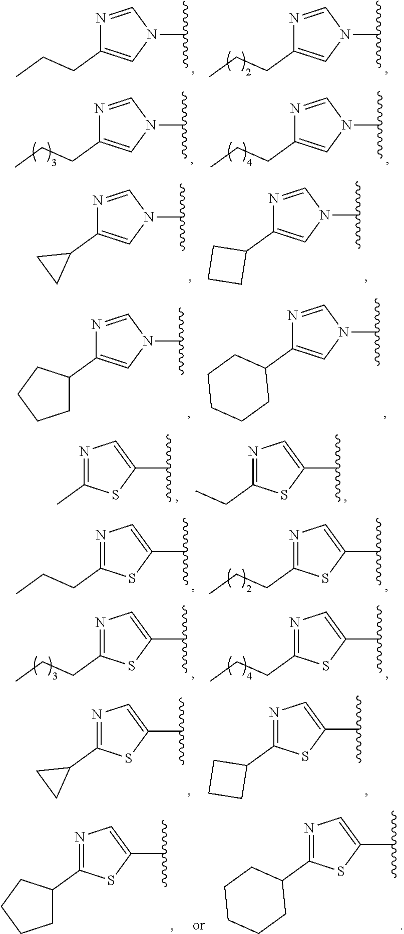

In some embodiments, R²⁵ is

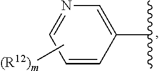

wherein R¹² is halo, C₁-C₆alkyl, or C₃-C₆cycloalkyl; and
m is 1 or 2.

In some embodiments, R²⁵ is selected from a group consisting of unsubstituted pyrazole, unsubstituted imidazole, unsubstituted thiazole, and unsubstituted pyridine.

In some embodiments, R²⁵ is selected from a group consisting of pyrimidine, pyrazine, and pyridazine; wherein pyrimidine, pyrazine, and pyridazine are optionally substituted with one or two substituents selected from the group consisting of halo, C₁₋₆alkyl, and C₃₋₈cycloalkyl.

In some embodiments, R²⁵ is selected from a group consisting of halogen, —OR⁶, —N(R⁶)₂, C₁₋₆alkyl, pyrazole, imidazole, thiazole, and pyridine; wherein pyrazole, imidazole, thiazole, and pyridine are optionally substituted with one or two substituents selected from the group consisting of halo, C₁₋₆alkyl, and C₃₋₈cycloalkyl.

In some embodiments, R²⁵ is selected from a group consisting of halogen, —OR⁶, —N(R⁶)₂, C₁₋₆alkyl, and unsubstituted pyridine.

In some embodiments, R²⁵ is —C(═O)N(R⁶)₂ and each R⁶ is independently selected from the group consisting of hydrogen, C₁-C₆alkyl, —C₁-C₆alkyl-O—C₁-C₆alkyl, —C₁-C₆alkyl-C₂₋₉heterocycle, —C₁-C₆alkyl-C₂₋₉heteroaryl, C₃-C₈cycloalkyl, and C₂₋₉heterocycle.

In some embodiments, R²⁵ is

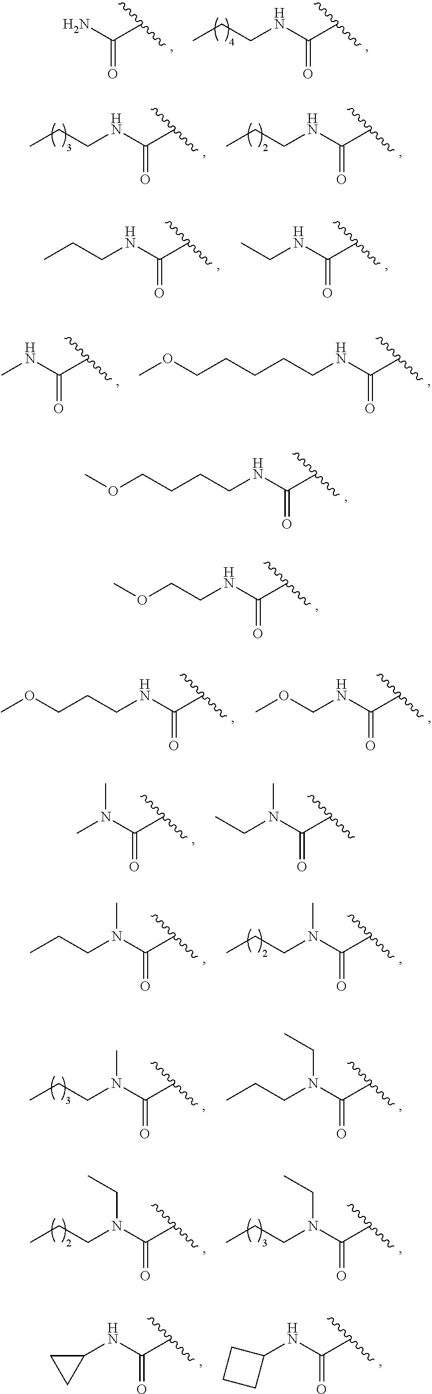

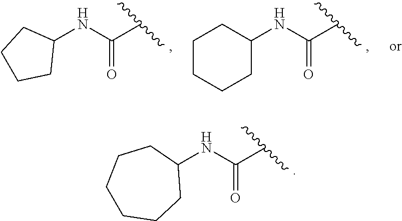 [SMILES: *C(=O)NC1CCCC1 ; *C(=O)NC1CCCCC1 ; *C(=O)NC1CCCCCC1]

In some embodiments, R²⁵ is

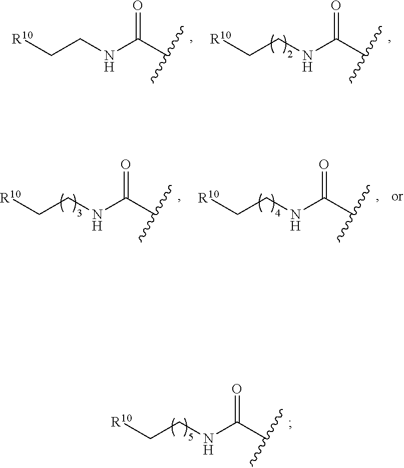

wherein R¹⁰ is a heteroaryl.

In some embodiments, R²⁵ is —C(═O)N(R⁶)₂ and two R⁶ on the same heteroatom are taken together with that heteroatom to which they are attached to form a C₂₋₉heterocycle or a C₂₋₉heteroaryl, wherein C₂₋₉heterocycle or C₂₋₉heteroaryl are optionally substituted with one, two, or three substituents selected from the group consisting of halo, —OR⁸, —SR⁸, —N(R⁸)₂, —C₁₋₆alkyl, —O—C₁₋₆alkyl, —C(═O)R¹⁴, —C(═O)OR¹³, and —N(R¹³)C(═O)R¹⁴.

In some embodiments, R²⁵ is

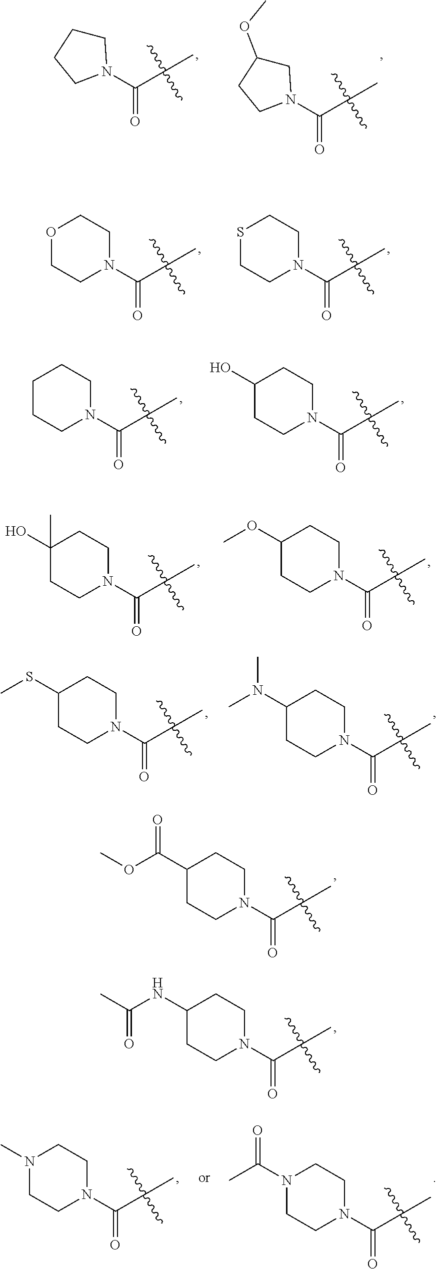

In some embodiments, R²⁵ is —C(═O)N(R⁶)₂ and two R⁶ are taken together with that heteroatom to which they are attached to form a C₂₋₉heterocycle or a C₂₋₉heteroaryl.

In some embodiments, R²⁵ is

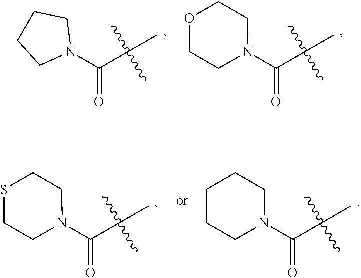

In some embodiments, R²⁵ is —OR⁶ and R⁶ is selected from the group consisting of C₁-C₆alkyl, —C₁-C₆alkyl-O—C₁-C₆alkyl, and —C₁-C₆alkyl-C₂₋₉heterocycle.

In some embodiments, n is 0. In some embodiments, n is 1. In some embodiments, n is 2.

In some embodiments, Z is C(R⁹)₂. In some embodiments, Z is C(R⁹)₂ and each R⁹ is H.

In some embodiments, Z is N(R⁸). In some embodiments, Z is N(R′) and each R⁸ is H. In some embodiments, Z is N(R′) and each R⁸ is C₁-C₆alkyl.

In some embodiments,

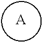

is

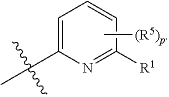

In some embodiments, p is 0.

In some embodiments,

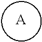

is

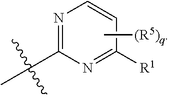

In some embodiments,

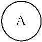

is

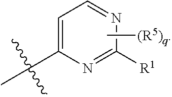

In some embodiments, q is 0.

In some embodiments, R¹ is

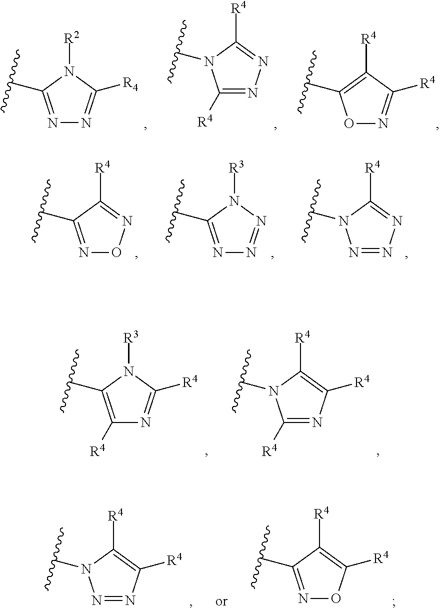

wherein R² is C₃₋₆cycloalkyl; R³ is selected from a group consisting of hydrogen C₁₋₆alkyl and C₃₋₆cycloalkyl; each R⁴ is independently selected from a group consisting of hydrogen, halogen, C₁₋₆ alkyl, and C₃₋₆cycloalkyl; or one R⁴ and another R², R³, or R⁴, together with the atoms to which they are attached, form a 5- or 6-membered ring that is optionally containing one or two heteroatoms selected from O, N, and S; wherein the 5- or 6-membered ring is saturated, unsaturated or aromatic; and wherein the 5- or 6-membered ring is optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R¹ is

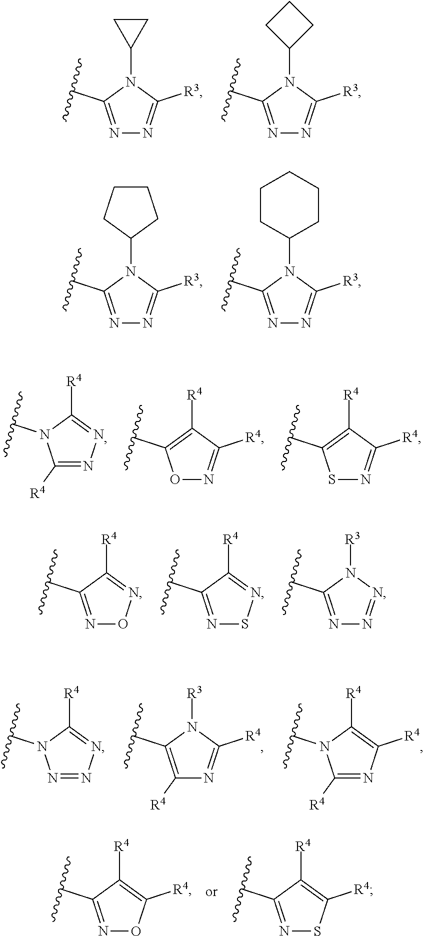

wherein R³ is selected from a group consisting of C₁₋₆alkyl and C₃₋₆cycloalkyl; and
each R⁴ is independently selected from a group consisting of hydrogen, halogen, C₁₋₆alkyl, and C₃₋₆cycloalkyl.

In some embodiments, R¹ is

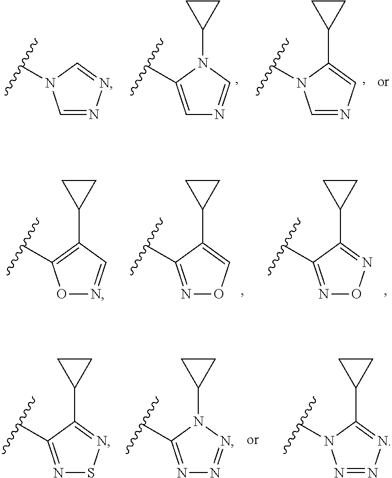

In some embodiments, R¹ is

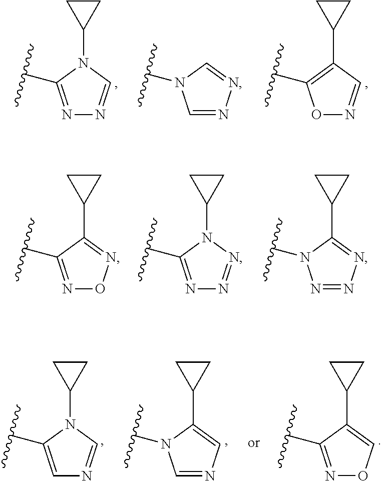

In some embodiments, R¹ is

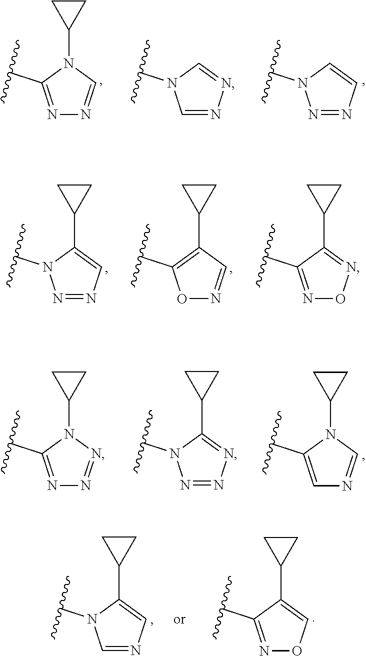

In some embodiments, R¹ is

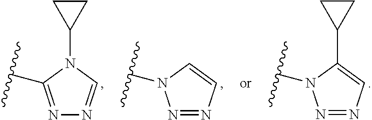

In some embodiments, R¹ is

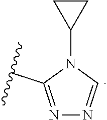 [SMILES: *c1nncn1C1CC1 ; "."]

In some embodiments, R¹ is

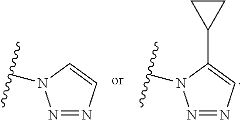 [SMILES: *n1ccnn1 ; *n1nncc1C1CC1]

In some embodiments, R¹ is

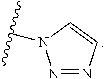 [SMILES: *n1ccnn1]

In some embodiments, R¹ is

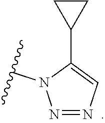 [SMILES: *n1nncc1C1CC1]

In some embodiments, R¹ is

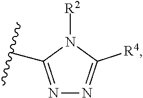

wherein R² and R⁴, together with the atoms to which they are attached, form a 5- or 6-membered ring that is optionally containing one or two heteroatoms selected from O, N, and S; wherein the 5- or 6-membered ring is saturated, unsaturated or aromatic; and wherein the 5- or 6-membered ring is optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R¹ is

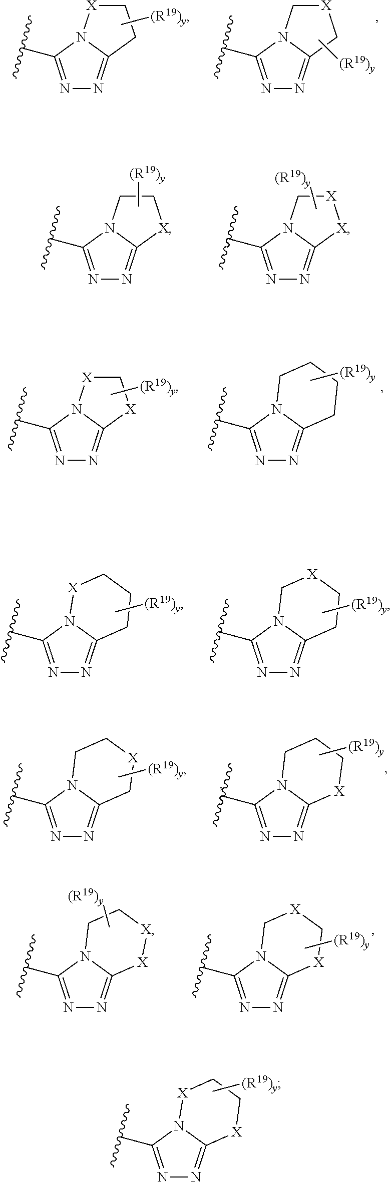

wherein X is 0, N or S; each R¹⁹ is independently halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³; and y is 0, 1, 2, 3 or 4.

In some embodiments, R¹ is

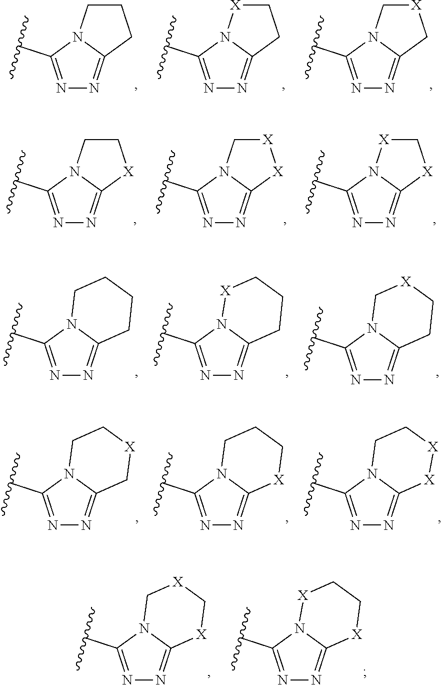

wherein X is 0, N or S.

In some embodiments, R¹ is

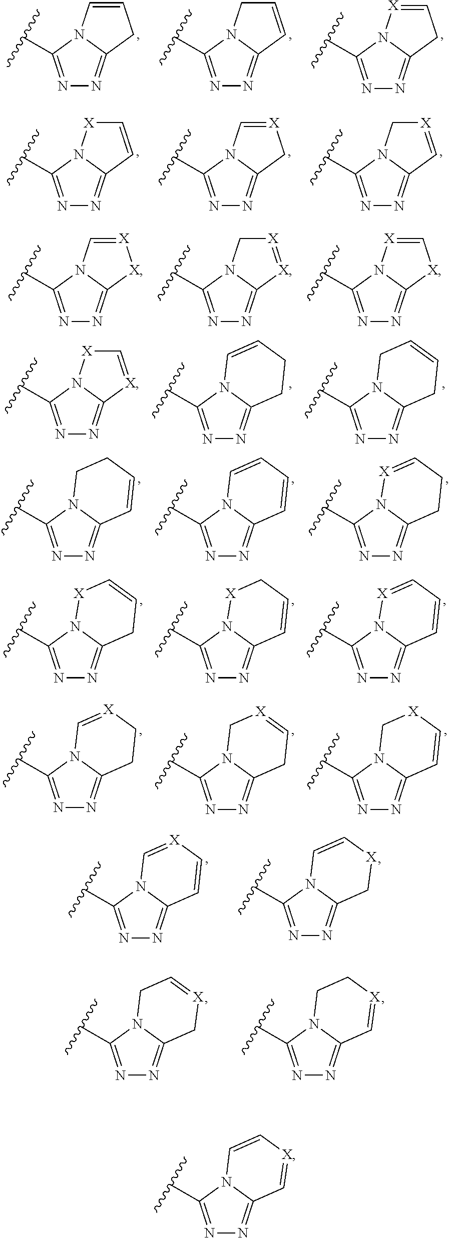

wherein X is N.

In some embodiments, R¹ is

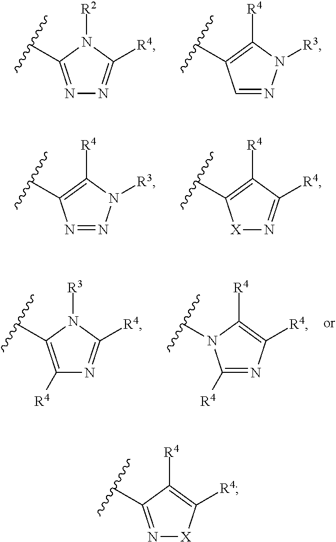

### X is O or S; and

one R⁴ and another R², R³, or R⁴, together with the atoms to which they are attached, form a 5- or 6-membered ring that is optionally containing one or two heteroatoms selected from O, N, and S; wherein the 5- or 6-membered ring is saturated, unsaturated or aromatic; and wherein the 5- or 6-membered ring is optionally substituted with one, two, or three substituents selected from the group consisting of halo, —CN, C₁₋₆alkyl, —C₁₋₆alkyl-OH, C₁₋₆haloalkyl, C₃₋₈cycloalkyl, C₂₋₉heterocycle, C₆₋₁₀aryl, C₁₋₉heteroaryl, —C(═O)R¹⁴, —C(═O)OR¹³, —C(═O)N(R¹³)₂, —S(═O)R¹⁴, —S(═O)₂R¹³, —S(═O)₂—N(R¹³)₂, —N(R¹³)₂, —N(R¹³)C(═O)R¹⁴, and —N(R¹³)S(═O)₂R¹³.

In some embodiments, R¹ is

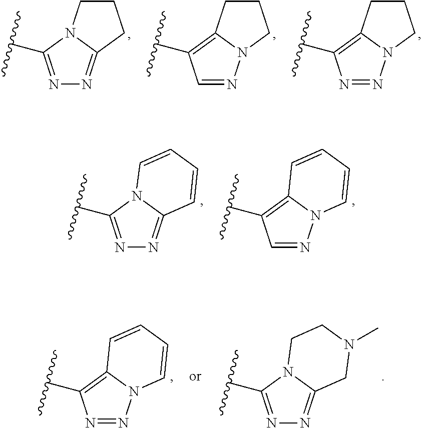

In some embodiments, R¹ is

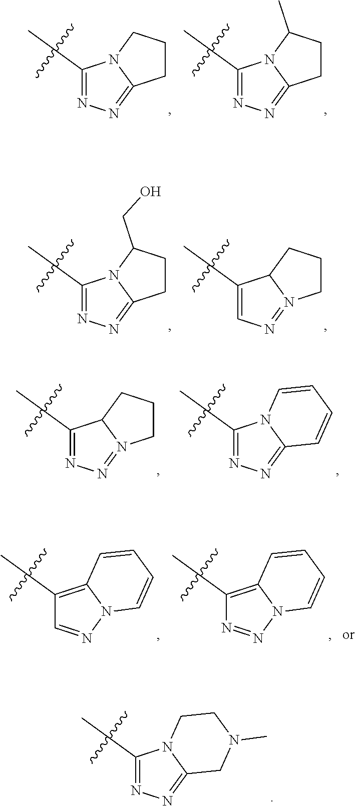

In some embodiments, presented herein are pharmaceutical compositions comprising a compound of Formula I, Formula II, Formula IIa, or Formula III, or a pharmaceutically acceptable salt or solvate thereof, and at least one pharmaceutically acceptable excipient.

In some embodiments is a method for treating an organ disease or disorder in a subject in need thereof comprising administering to the subject a therapeutically effective amount of a compound of Formula I, Formula II, Formula IIa, or Formula III, or a pharmaceutically acceptable salt or solvate thereof. In some embodiments is a method for treating an organ disease or disorder in a subject in need thereof comprising administering to the subject a therapeutically effective amount of a compound of Formula I, or a pharmaceutically acceptable salt or solvate thereof. In some embodiments is a method for treating an organ disease or disorder in a subject in need thereof comprising administering to the subject a therapeutically effective amount of a compound of Formula II, or a pharmaceutically acceptable salt or solvate thereof. In some embodiments is a method for treating an organ disease or disorder in a subject in need thereof comprising administering to the subject a therapeutically effective amount of a compound of Formula IIa, or a pharmaceutically acceptable salt or solvate thereof. In some embodiments is a method for treating an organ disease or disorder in a subject in need thereof comprising administering to the subject a therapeutically effective amount of a compound of Formula III, or a pharmaceutically acceptable salt or solvate thereof.

In some embodiments is a method for treating an organ disease or disorder in a subject in need thereof comprising administering to the subject a therapeutically effective amount of a compound of Formula I, Formula II, Formula IIa, or Formula III, or a pharmaceutically acceptable salt or solvate thereof, wherein the organ disease or disorder is selected from liver disease or disorder, heart disease or disorder, kidney disease or disorder, pancreas disease or disorder, spleen disease or disorder, and skin disease or disorder. In some embodiments, the organ disease or disorder is selected from liver disease or disorder, heart disease or disorder, and kidney disease or disorder. In some embodiments, the organ disease or disorder is selected from liver disease or disorder and kidney disease or disorder.

In some embodiments is a method for treating an organ disease or disorder in a subject in need thereof comprising administering to the subject a therapeutically effective amount of a compound of Formula I, or a pharmaceutically acceptable salt or solvate thereof, wherein the organ disease or disorder is selected from liver disease or disorder, heart disease or disorder, kidney disease or disorder, pancreas disease or disorder, spleen disease or disorder, and skin disease or disorder. In some embodiments, the organ disease or disorder is selected from liver disease or disorder, heart disease or disorder, and kidney disease or disorder. In some embodiments, the organ disease or disorder is selected from liver disease or disorder and kidney disease or disorder.

In some embodiments is a method for treating an organ disease or disorder in a subject in need thereof comprising administering to the subject a therapeutically effective amount of a compound of Formula II, or a pharmaceutically acceptable salt or solvate thereof, wherein the organ disease or disorder is selected from liver disease or disorder, heart disease or disorder, kidney disease or disorder, pancreas disease or disorder, spleen disease or disorder, and skin disease or disorder. In some embodiments, the organ disease or disorder is selected from liver disease or disorder, heart disease or disorder, and kidney disease or disorder. In some embodiments, the organ disease or disorder is selected from liver disease or disorder and kidney disease or disorder.

In some embodiments is a method for treating an organ disease or disorder in a subject in need thereof comprising administering to the subject a therapeutically effective amount of a compound of Formula IIa, or a pharmaceutically acceptable salt or solvate thereof, wherein the organ disease or disorder is selected from liver disease or disorder, heart disease or disorder, kidney disease or disorder, pancreas disease or disorder, spleen disease or disorder, and skin disease or disorder. In some embodiments, the organ disease or disorder is selected from liver disease or disorder, heart disease or disorder, and kidney disease or disorder. In some embodiments, the organ disease or disorder is selected from liver disease or disorder and kidney disease or disorder.

In some embodiments is a method for treating an organ disease or disorder in a subject in need thereof comprising administering to the subject a therapeutically effective amount of a compound of Formula III, or a pharmaceutically acceptable salt or solvate thereof, wherein the organ disease or disorder is selected from liver disease or disorder, heart disease or disorder, kidney disease or disorder, pancreas disease or disorder, spleen disease or disorder, and skin disease or disorder. In some embodiments, the organ disease or disorder is selected from liver disease or disorder, heart disease or disorder, and kidney disease or disorder. In some embodiments, the organ disease or disorder is selected from liver disease or disorder and kidney disease or disorder.

In some embodiments is a method for treating an organ disease or disorder in a subject in need thereof comprising administering to the subject a therapeutically effective amount of

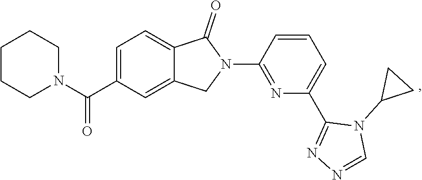 [SMILES: O=C(c1ccc2c(c1)CN(c1cccc(-c3nncn3C3CC3)n1)C2=O)N1CCCCC1]

or a pharmaceutically acceptable salt or solvate thereof.

In some embodiments is a method for treating an organ disease or disorder in a subject in need thereof comprising administering to the subject a therapeutically effective amount of

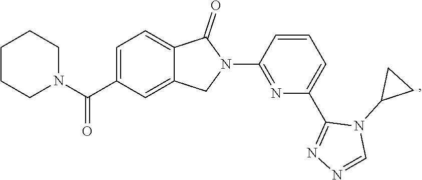 [SMILES: O=C(c1ccc2c(c1)CN(c1cccc(-c3nncn3C3CC3)n1)C2=O)N1CCCCC1]

or a pharmaceutically acceptable salt or solvate thereof, wherein the organ disease or disorder is selected from liver disease or disorder, heart disease or disorder, kidney disease or disorder, pancreas disease or disorder, spleen disease or disorder, and skin disease or disorder. In some embodiments, the organ disease or disorder is selected from liver disease or disorder, heart disease or disorder, and kidney disease or disorder. In some embodiments, the organ disease or disorder is the liver disease or disorder, heart disease or disorder, or kidney disease or disorder described herein.

In some embodiments is a method for treating an organ disease or disorder in a subject in need thereof comprising administering to the subject a therapeutically effective amount of

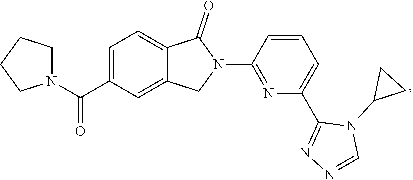 [SMILES: O=C(c1ccc2c(c1)CN(c1cccc(-c3nncn3C3CC3)n1)C2=O)N1CCCC1]

or a pharmaceutically acceptable salt or solvate thereof.

In some embodiments is a method for treating an organ disease or disorder in a subject in need thereof comprising administering to the subject a therapeutically effective amount of

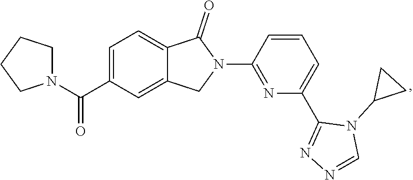 [SMILES: O=C(c1ccc2c(c1)CN(c1cccc(-c3nncn3C3CC3)n1)C2=O)N1CCCC1]

or a pharmaceutically acceptable salt or solvate thereof, wherein the organ disease or disorder is selected from organ disease or disorder is selected from liver disease or disorder, heart disease or disorder, kidney disease or disorder, pancreas disease or disorder, spleen disease or disorder, and skin disease or disorder. In some embodiments, the organ disease or disorder is selected from liver disease or disorder, heart disease or disorder, and kidney disease or disorder. In some embodiments, the organ disease or disorder is the liver disease or disorder, heart disease or disorder, or kidney disease or disorder described herein.

In some embodiments is a method for treating an organ disease or disorder in a subject in need thereof comprising administering to the subject a therapeutically effective amount of

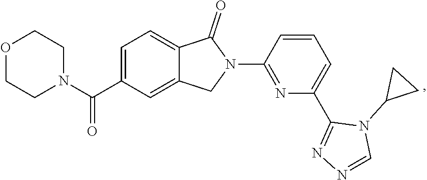 [SMILES: O=C(c1ccc2c(c1)CN(c1cccc(-c3nncn3C3CC3)n1)C2=O)N1CCOCC1]

or a pharmaceutically acceptable salt or solvate thereof.

In some embodiments is a method for treating an organ disease or disorder in a subject in need thereof comprising administering to the subject a therapeutically effective amount of

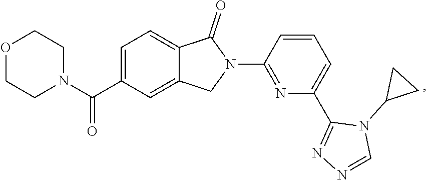 [SMILES: O=C(c1ccc2c(c1)CN(c1cccc(-c3nncn3C3CC3)n1)C2=O)N1CCOCC1]

or a pharmaceutically acceptable salt or solvate thereof, wherein the organ disease or disorder is selected from liver disease or disorder, heart disease or disorder, kidney disease or disorder, pancreas disease or disorder, spleen disease or disorder, and skin disease or disorder. In some embodiments, the organ disease or disorder is selected from liver disease or disorder, heart disease or disorder, and kidney disease or disorder. In some embodiments, the organ disease or disorder is the liver disease or disorder, heart disease or disorder, or kidney disease or disorder described herein.

In some embodiments is a method for treating an organ disease or disorder in a subject in need thereof comprising administering to the subject a therapeutically effective amount of

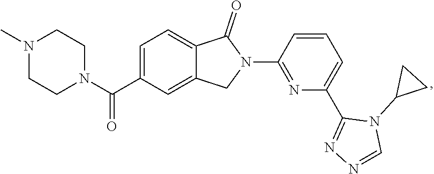 [SMILES: CN1CCN(C(=O)c2ccc3c(c2)CN(c2cccc(-c4nncn4C4CC4)n2)C3=O)CC1]

or a pharmaceutically acceptable salt or solvate thereof.

In some embodiments is a method for treating an organ disease or disorder in a subject in need thereof comprising administering to the subject a therapeutically effective amount of

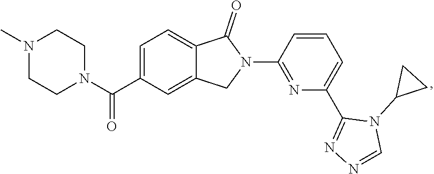 [SMILES: CN1CCN(C(=O)c2ccc3c(c2)CN(c2cccc(-c4nncn4C4CC4)n2)C3=O)CC1]

or a pharmaceutically acceptable salt or solvate thereof, wherein the organ disease or disorder is selected from liver disease or disorder, heart disease or disorder, kidney disease or disorder, pancreas disease or disorder, spleen disease or disorder, and skin disease or disorder. In some embodiments, the organ disease or disorder is selected from liver disease or disorder, heart disease or disorder, and kidney disease or disorder. In some embodiments, the organ disease or disorder is the liver disease or disorder, heart disease or disorder, or kidney disease or disorder described herein.

In some embodiments is a method for treating an organ disease or disorder in a subject in need thereof comprising administering to the subject a therapeutically effective amount of

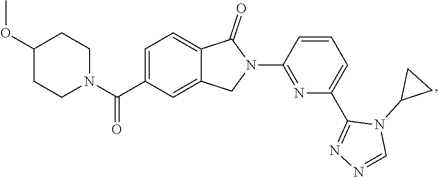 [SMILES: COC1CCN(C(=O)c2ccc3c(c2)CN(c2cccc(-c4nncn4C4CC4)n2)C3=O)CC1]

or a pharmaceutically acceptable salt or solvate thereof.

In some embodiments is a method for treating an organ disease or disorder in a subject in need thereof comprising administering to the subject a therapeutically effective amount of

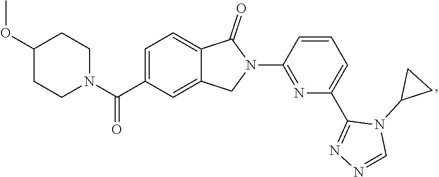 [SMILES: COC1CCN(C(=O)c2ccc3c(c2)CN(c2cccc(-c4nncn4C4CC4)n2)C3=O)CC1]

or a pharmaceutically acceptable salt or solvate thereof, wherein organ disease or disorder is selected from liver disease or disorder, heart disease or disorder, kidney disease or disorder, pancreas disease or disorder, spleen disease or disorder, and skin disease or disorder. In some embodiments, the organ disease or disorder is selected from liver disease or disorder, heart disease or disorder, and kidney disease or disorder. In some embodiments, the organ disease or disorder is the liver disease or disorder, heart disease or disorder, or kidney disease or disorder described herein.

In some embodiments is a method for treating an organ disease or disorder in a subject in need thereof comprising administering to the subject a therapeutically effective amount of a compound described herein, or a pharmaceutically acceptable salt or solvate thereof.

In some embodiments is a method for treating an organ disease or disorder in a subject in need thereof comprising administering to the subject a therapeutically effective amount of a compound described herein, or a pharmaceutically acceptable salt or solvate thereof, wherein the organ disease or disorder is selected from liver disease or disorder, heart disease or disorder, kidney disease or disorder, pancreas disease or disorder, spleen disease or disorder, and skin disease or disorder. In some embodiments, the organ disease or disorder is selected from liver disease or disorder, heart disease or disorder, and kidney disease or disorder. In some embodiments, the organ disease or disorder is the liver disease or disorder, heart disease or disorder, or kidney disease or disorder described herein.

In some embodiments described herein is a method for treating an organ disease or disorder in a subject in need thereof comprising administering to the subject a therapeutically effective amount of a compound selected from Table 1.

In some embodiments described herein is a method for treating an organ disease or disorder in a subject in need thereof comprising administering to the subject a therapeutically effective amount of a compound selected from Table 1, wherein the organ disease or disorder is selected from liver disease or disorder, heart disease or disorder, kidney disease or disorder, pancreas disease or disorder, spleen disease or disorder, and skin disease or disorder. In some embodiments, the organ disease or disorder is selected from liver disease or disorder, heart disease or disorder, and kidney disease or disorder.

TABLE 1

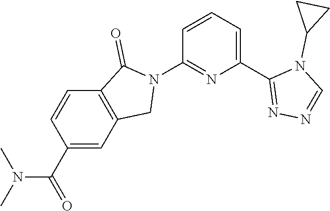 [SMILES: CN(C)C(=O)c1ccc2c(c1)CN(c1cccc(-c3nncn3C3CC3)n1)C2=O]

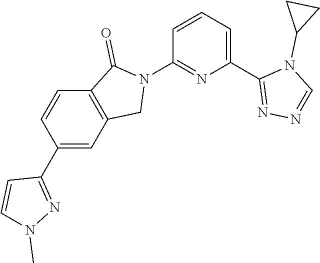 [SMILES: Cn1ccc(-c2ccc3c(c2)CN(c2cccc(-c4nncn4C4CC4)n2)C3=O)n1]

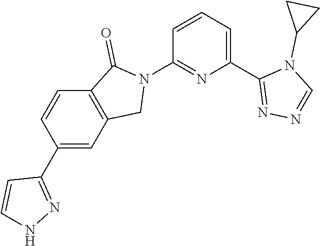 [SMILES: O=C1c2ccc(-c3cc[nH]n3)cc2CN1c1cccc(-c2nncn2C2CC2)n1]

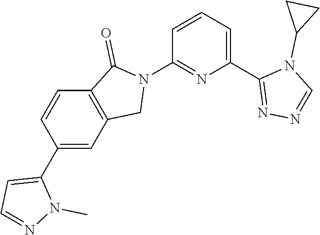 [SMILES: Cn1nccc1-c1ccc2c(c1)CN(c1cccc(-c3nncn3C3CC3)n1)C2=O]

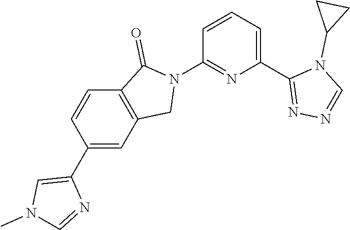 [SMILES: Cn1cnc(-c2ccc3c(c2)CN(c2cccc(-c4nncn4C4CC4)n2)C3=O)c1]

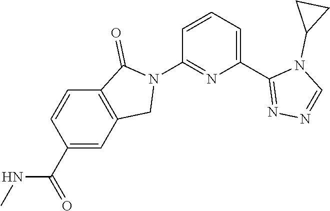 [SMILES: CNC(=O)c1ccc2c(c1)CN(c1cccc(-c3nncn3C3CC3)n1)C2=O]

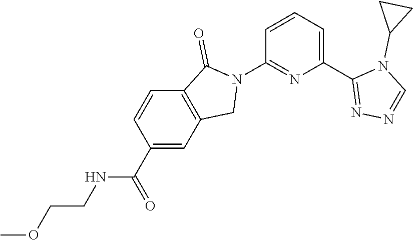 [SMILES: COCCNC(=O)c1ccc2c(c1)CN(c1cccc(-c3nncn3C3CC3)n1)C2=O]

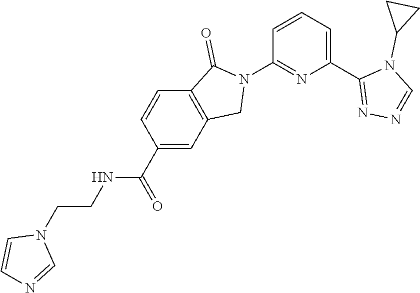 [SMILES: O=C(NCCn1ccnc1)c1ccc2c(c1)CN(c1cccc(-c3nncn3C3CC3)n1)C2=O]

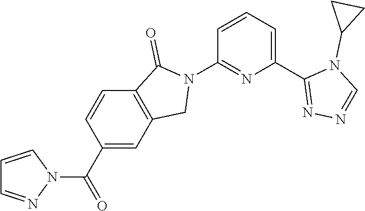 [SMILES: O=C1c2ccc(C(=O)n3cccn3)cc2CN1c1cccc(-c2nncn2C2CC2)n1]

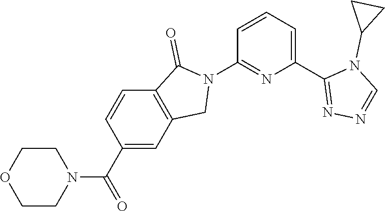 [SMILES: O=C(c1ccc2c(c1)CN(c1cccc(-c3nncn3C3CC3)n1)C2=O)N1CCOCC1]

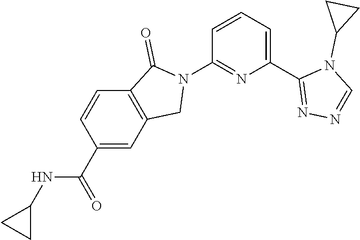 [SMILES: O=C(NC1CC1)c1ccc2c(c1)CN(c1cccc(-c3nncn3C3CC3)n1)C2=O]

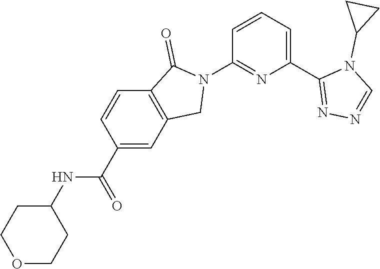 [SMILES: O=C(NC1CCOCC1)c1ccc2c(c1)CN(c1cccc(-c3nncn3C3CC3)n1)C2=O]

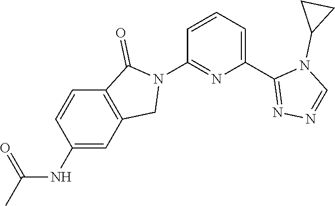 [SMILES: CC(=O)Nc1ccc2c(c1)CN(c1cccc(-c3nncn3C3CC3)n1)C2=O]

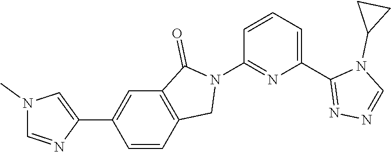 [SMILES: Cn1cnc(-c2ccc3c(c2)C(=O)N(c2cccc(-c4nncn4C4CC4)n2)C3)c1]

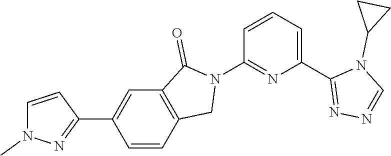 [SMILES: Cn1ccc(-c2ccc3c(c2)C(=O)N(c2cccc(-c4nncn4C4CC4)n2)C3)n1]

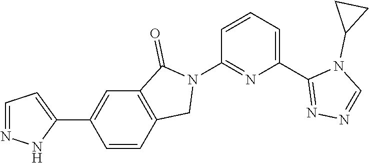 [SMILES: O=C1c2cc(-c3ccn[nH]3)ccc2CN1c1cccc(-c2nncn2C2CC2)n1]

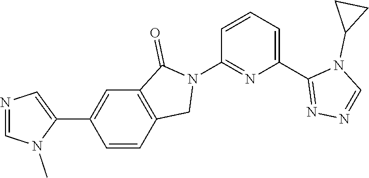 [SMILES: Cn1cncc1-c1ccc2c(c1)C(=O)N(c1cccc(-c3nncn3C3CC3)n1)C2]

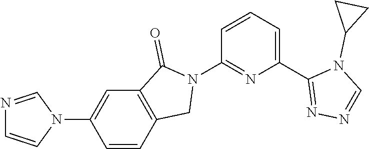 [SMILES: O=C1c2cc(-n3ccnc3)ccc2CN1c1cccc(-c2nncn2C2CC2)n1]

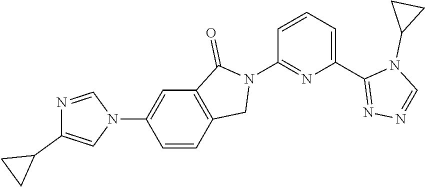 [SMILES: O=C1c2cc(-n3cnc(C4CC4)c3)ccc2CN1c1cccc(-c2nncn2C2CC2)n1]

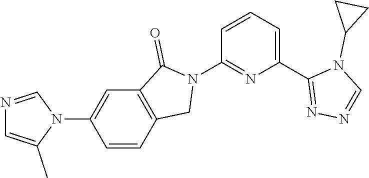 [SMILES: Cc1cncn1-c1ccc2c(c1)C(=O)N(c1cccc(-c3nncn3C3CC3)n1)C2]

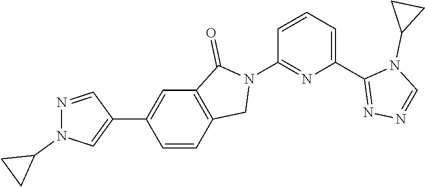 [SMILES: O=C1c2cc(-c3cnn(C4CC4)c3)ccc2CN1c1cccc(-c2nncn2C2CC2)n1]

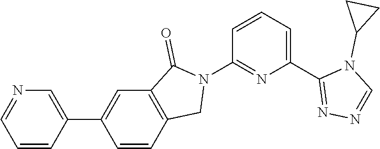 [SMILES: O=C1c2cc(-c3cccnc3)ccc2CN1c1cccc(-c2nncn2C2CC2)n1]

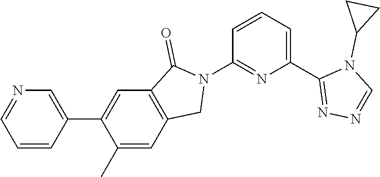 [SMILES: Cc1cc2c(cc1-c1cccnc1)C(=O)N(c1cccc(-c3nncn3C3CC3)n1)C2]

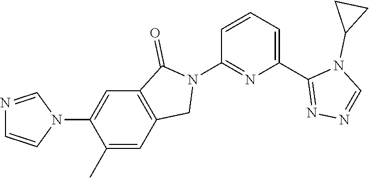 [SMILES: Cc1cc2c(cc1-n1ccnc1)C(=O)N(c1cccc(-c3nncn3C3CC3)n1)C2]

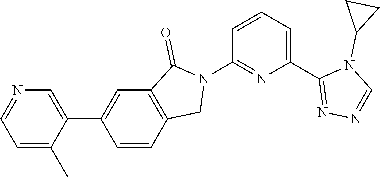 [SMILES: Cc1ccncc1-c1ccc2c(c1)C(=O)N(c1cccc(-c3nncn3C3CC3)n1)C2]

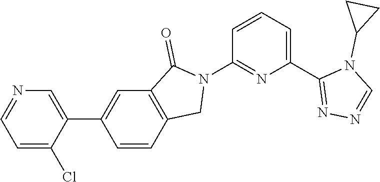 [SMILES: O=C1c2cc(-c3cnccc3Cl)ccc2CN1c1cccc(-c2nncn2C2CC2)n1]

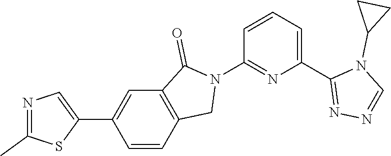 [SMILES: Cc1ncc(-c2ccc3c(c2)C(=O)N(c2cccc(-c4nncn4C4CC4)n2)C3)s1]

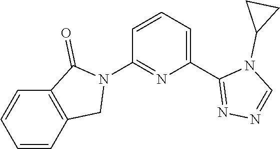 [SMILES: O=C1c2ccccc2CN1c1cccc(-c2nncn2C2CC2)n1]

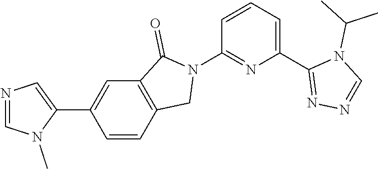 [SMILES: CC(C)n1cnnc1-c1cccc(N2Cc3ccc(-c4cncn4C)cc3C2=O)n1]

In some embodiments, the organ disease or disorder is liver disease or disorder. In some embodiments, the liver disease or disorder is selected from a liver injury, a drug-induced liver injury, a liver failure, a fulminant or acute liver failure, and an acute-on-chronic liver failure. In some embodiments, the fulminant liver failure is due to a drug overdose, due to acetaminophen overdose, due to poisoning with wild mushroom Amanita phalloides, or due to carbon tetrachloride disorder. In some embodiments, the acute liver failure is due to poisoning, due to surgical ischemia-reperfusion injury, due to vascular diseases, due to metabolic diseases, due to pregnancy, or due to septic shock. In some embodiments, the acute-on-chronic liver failure is due to alcoholic hepatitis, due to autoimmune hepatitis, due to viral hepatitis, or due to cirrhosis.

In some embodiments, the liver disease or disorder is selected from drug-induced liver injury, fulminant liver failure due to acetaminophen overdose, fulminant liver failure due to poisoning with wild mushroom Amanita phalloides or carbon tetrachloride disorder, acute liver failure due to poisoning with herbal supplements, including kava, ephedra, skullcap and pennyroyal, acute liver failure due to surgical ischemia-reperfusion injury (liver transplant, partial resection), acute liver failure due to vascular diseases, such as Budd-Chiari syndrome, acute liver failure due to metabolic disease such as Wilson's disease, acute fatty liver of pregnancy, acute liver failure due to septic shock, acute-on-chronic liver failure due to alcoholic hepatitis, acute-on-chronic liver failure due to autoimmune hepatitis, and acute-on-chronic liver failure due to viral hepatitis such as hepatitis A, hepatitis B and hepatitis E, Epstein-Barr virus, cytomegalovirus and herpes simplex virus.

In some embodiments, the liver disease or disorder is drug-induced liver injury. In some embodiments, the liver disease or disorder is fulminant liver failure due to acetaminophen overdose. In some embodiments, the liver disease or disorder is fulminant liver failure due to poisoning with wild mushroom Amanita phalloides or carbon tetrachloride disorder. In some embodiments, the liver disease or disorder is acute liver failure due to poisoning with herbal supplements, including kava, ephedra, skullcap and pennyroyal. In some embodiments, the liver disease or disorder is acute liver failure due to surgical ischemia-reperfusion injury (liver transplant, partial resection). In some embodiments, the liver disease or disorder is acute liver failure due to vascular diseases, such as Budd-Chiari syndrome. In some embodiments, the liver disease or disorder is acute liver failure due to metabolic disease such as Wilson's disease and acute fatty liver of pregnancy. In some embodiments, the liver disease or disorder is acute liver failure due to septic shock. In some embodiments, the liver disease or disorder is acute-on-chronic liver failure due to alcoholic hepatitis. In some embodiments, the liver disease or disorder is acute-on-chronic liver failure due to autoimmune hepatitis. In some embodiments, the liver disease or disorder is acute-on-chronic liver failure due to viral hepatitis such as hepatitis A, hepatitis B and hepatitis E, Epstein-Barr virus, cytomegalovirus and herpes simplex virus.

In some embodiments, the organ disease or disorder is heart disease or disorder. In some embodiments, the heart disease or disorder is selected from ischemia reperfusion injury, ischemia, coronary artery disease, cardiovascular dysfunction from sepsis, drug-induced cardiotoxicity, viral myocarditis, and complications due to heart transplant. In some embodiments, the heart disease or disorder is ischemia reperfusion injury. In some embodiments, the ischemia reperfusion injury is due to acute ST-segment elevation myocardial infarction (STEMI), percutaneous coronary intervention (PCI), coronary artery bypass surgery (CABG), cardiopulmonary bypass and extracorporeal circulation, or a combination thereof. In some embodiments, the heart disease or disorder is ischemia. Examples of ischemia includes but is not limited to coronary artery disease and myocardial ischemia. In some embodiments, the heart disease or disorder is coronary artery disease. In some embodiments, the heart disease or disorder is cardiovascular dysfunction from sepsis. In some embodiments, the heart disease or disorder is drug-induced cardiotoxicity. In some embodiments, the drug toxicity is due to doxorubicin. In some embodiments, the heart disease or disorder is viral myocarditis. In some embodiments, the heart disease or disorder is complications due to heart transplant.

In some embodiments, the organ disease or disorder is kidney disease or disorder. In some embodiments, the kidney disease or disorder is acute kidney injury. In some embodiments acute kidney injury is due to hypovolemia, hypotension, renal vasoconstriction, glomerular efferent arteriolar vasodilation, prolonged renal ischemia, prolonged renal sepsis, prolonged renal nephrotoxins, kidney transplants, acute tubular necrosis, acute interstitial nephritis, glomerulonephritis, and intratubular obstruction. In some embodiments, hypovolemia is due to hemorrhage, severe burns, and gastrointestinal fluid losses such as diarrhea, vomiting, high ostomy output. In some embodiments, hypotension is due to decreased cardiac output, such as cardiogenic shock, massive pulmonary embolism, and acute coronary syndrome. In some embodiments, hypotension is due to systemic vasodilation such as septic shock, anaphylaxis, anesthesia administration, and hepatorenal syndrome. In some embodiments, renal vasoconstriction is due to non-steroidal anti-inflammatory drugs (NSAIDs), iodinated contrast, amphotericin B, calcineurin inhibitors, and hepatorenal syndrome. In some embodiments, glomerular efferent arteriolar vasodilation is due to ACE inhibitors and angiotensin receptor blockers. In some embodiments, acute tubular necrosis is due to ischemia from prolonged prerenal injury; drugs such as aminoglycosides, vancomycin, amphotericin B, pentamidine; rhabdomyolysis; and intravascular hemolysis. In some embodiments, acute interstitial nephritis is due to drugs such as beta-lactam antibiotics, penicillins, NSAIDs, proton pump inhibitors (PPIs), 5-ASA, infections, and autoimmune conditions (systemic lupus erythematosus (SLE), IgG related disease). In some embodiments, glomerulonephritis is due to anti-glomerular basement membrane disease, immune complex-mediated diseases such as SLE, post-infectious glomerulonephritis, cryoglobulinemia, IgA nephropathy, and Henoch-Schonlein purpura. In some embodiments, intratubular obstruction is due to monoclonal gammopathy seen in multiple myeloma, tumor lysis syndrome, toxins such as ethylene glycol.

In some embodiments, described herein is a method for treating an organ disease or disorder in a subject in need thereof comprising administering to the subject a therapeutically effective amount of a compound of Formula I, Formula II, Formula IIa, or Formula III, or a pharmaceutically acceptable salt or solvate thereof, wherein the compound is administered by injectable delivery. In some embodiments, described herein is a method for treating an organ disease or disorder in a subject in need thereof comprising administering to the subject a therapeutically effective amount of a compound of Formula I, Formula II, Formula IIa, or Formula III, or a pharmaceutically acceptable salt or solvate thereof, wherein the compound is administered by intravenous delivery, wherein the intravenous delivery is selected from bolus injection, intravenous drip, and infusion pump. In some embodiments, described herein is a method for treating an organ disease or disorder in a subject in need thereof comprising administering to the subject a therapeutically effective amount of a compound of Formula I, Formula II, Formula IIa, or Formula III, or a pharmaceutically acceptable salt or solvate thereof, wherein the compound is administered by intraperitoneal delivery.

In some embodiments, described herein is a method for treating an organ disease or disorder in a subject in need thereof comprising administering to the subject a therapeutically effective amount of a compound of Formula I, or a pharmaceutically acceptable salt or solvate thereof, wherein the compound is administered by injectable delivery. In some embodiments, described herein is a method for treating an organ disease or disorder in a subject in need thereof comprising administering to the subject a therapeutically effective amount of a compound of Formula I, or a pharmaceutically acceptable salt or solvate thereof, wherein the compound is administered by intravenous delivery, wherein the intravenous delivery is selected from bolus injection, intravenous drip, and infusion pump. In some embodiments, described herein is a method for treating an organ disease or disorder in a subject in need thereof comprising administering to the subject a therapeutically effective amount of a compound of Formula I, or a pharmaceutically acceptable salt or solvate thereof, wherein the compound is administered by intraperitoneal delivery.

In some embodiments, described herein is a method for treating an organ disease or disorder in a subject in need thereof comprising administering to the subject a therapeutically effective amount of a compound of Formula II, or a pharmaceutically acceptable salt or solvate thereof, wherein the compound is administered by injectable delivery. In some embodiments, described herein is a method for treating an organ disease or disorder in a subject in need thereof comprising administering to the subject a therapeutically effective amount of a compound of Formula II, or a pharmaceutically acceptable salt or solvate thereof, wherein the compound is administered by intravenous delivery, wherein the intravenous delivery is selected from bolus injection, intravenous drip, and infusion pump. In some embodiments, described herein is a method for treating an organ disease or disorder in a subject in need thereof comprising administering to the subject a therapeutically effective amount of a compound of Formula II, or a pharmaceutically acceptable salt or solvate thereof, wherein the compound is administered by intraperitoneal delivery.

In some embodiments, described herein is a method for treating an organ disease or disorder in a subject in need thereof comprising administering to the subject a therapeutically effective amount of a compound of Formula IIa, or a pharmaceutically acceptable salt or solvate thereof, wherein the compound is administered by injectable delivery. In some embodiments, described herein is a method for treating an organ disease or disorder in a subject in need thereof comprising administering to the subject a therapeutically effective amount of a compound of Formula IIa, or a pharmaceutically acceptable salt or solvate thereof, wherein the compound is administered by intravenous delivery, wherein the intravenous delivery is selected from bolus injection, intravenous drip, and infusion pump. In some embodiments, described herein is a method for treating an organ disease or disorder in a subject in need thereof comprising administering to the subject a therapeutically effective amount of a compound of Formula IIa, or a pharmaceutically acceptable salt or solvate thereof, wherein the compound is administered by intraperitoneal delivery.

In some embodiments, described herein is a method for treating an organ disease or disorder in a subject in need thereof comprising administering to the subject a therapeutically effective amount of a compound of Formula III, or a pharmaceutically acceptable salt or solvate thereof, wherein the compound is administered by injectable delivery. In some embodiments, described herein is a method for treating an organ disease or disorder in a subject in need thereof comprising administering to the subject a therapeutically effective amount of a compound of Formula III, or a pharmaceutically acceptable salt or solvate thereof, wherein the compound is administered by intravenous delivery, wherein the intravenous delivery is selected from bolus injection, intravenous drip, and infusion pump. In some embodiments, described herein is a method for treating an organ disease or disorder in a subject in need thereof comprising administering to the subject a therapeutically effective amount of a compound of Formula III, or a pharmaceutically acceptable salt or solvate thereof, wherein the compound is administered by intraperitoneal delivery.

### Synthesis of Compounds

The compounds used in the methods described herein are made according to procedures disclosed in US2018/0291002, which is herein incorporated by reference in its entirety, or by known organic synthesis techniques, starting from commercially available chemicals and/or from compounds described in the chemical literature. The starting material used for the synthesis of the compounds described herein are either synthesized or obtained from commercial sources, such as, but not limited to, Sigma-Aldrich, Fluka, Acros Organics, Alfa Aesar, and the like. The compounds described herein, and other related compounds having different substituents are synthesized using techniques and materials described herein or otherwise known, including those found in March, ADVANCED ORGANIC CHEMISTRY 4^{th} Ed., (Wiley 1992); Carey and Sundberg, ADVANCED ORGANIC CHEMISTRY 4^{th} Ed., Vols. A and B (Plenum 2000, 2001), and Green and Wuts, PROTECTIVE GROUPS IN ORGANIC SYNTHESIS 3^{rd} Ed., (Wiley 1999). General methods for the preparation of compounds can be modified by the use of appropriate reagents and conditions for the introduction of the various moieties found in the formulae as provided herein. A detailed description of techniques applicable to the creation of protecting groups and their removal are described in Greene and Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

### Further Forms of Compounds

In one aspect, compounds described herein possess one or more stereocenters and each stereocenter exists independently in either the R or S configuration. The compounds presented herein include all diastereomeric, enantiomeric, and epimeric forms as well as the appropriate mixtures thereof. The compounds and methods provided herein include all cis-, trans-, syn-, anti-, entgegen (E), and zusammen (Z) isomers as well as the appropriate mixtures thereof. In certain embodiments, compounds described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In some embodiments, stereoisomers are obtained by stereoselective synthesis.

“Pharmaceutically acceptable,” as used herein, refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The term “pharmaceutically acceptable salt” refers to a formulation of a compound that does not cause significant irritation to an organism to which it is administered and does not abrogate the biological activity and properties of the compound. In some embodiments, pharmaceutically acceptable salts are obtained by reacting a compound described herein with acids.

Pharmaceutically acceptable salts are also obtained by reacting a compound described herein with a base to form a salt.

Compounds described herein may be formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid to form a salt such as, for example, a hydrochloric acid salt, a hydrobromic acid salt, a sulfuric acid salt, a phosphoric acid salt, a metaphosphoric acid salt, and the like; or with an organic acid to form a salt such as, for example, an acetic acid salt, a propionic acid salt, a hexanoic acid salt, a cyclopentanepropionic acid salt, a glycolic acid salt, a pyruvic acid salt, a lactic acid salt, a malonic acid salt, a succinic acid salt, a malic acid salt, a maleic acid salt, a fumaric acid salt, a trifluoroacetic acid salt, a tartaric acid salt, a citric acid salt, a benzoic acid salt, a 3-(4-hydroxybenzoyl)benzoic acid salt, a cinnamic acid salt, a mandelic acid salt, a methanesulfonic acid salt, an ethanesulfonic acid salt, a 1,2-ethanedisulfonic acid salt, a 2-hydroxyethanesulfonic acid salt, a benzenesulfonic acid salt, a toluenesulfonic acid salt, a 2-naphthalenesulfonic acid salt, a 4-methylbicyclo-[2.2.2]oct-2-ene-1-carboxylic acid salt, a glucoheptonic acid salt, a 4,4′-methylenebis-(3-hydroxy-2-ene-1-carboxylic acid) salt, a 3-phenylpropionic acid salt, a trimethylacetic acid salt, a tertiary butylacetic acid salt, a lauryl sulfuric acid salt, a gluconic acid salt, a glutamic acid salt, a hydroxynaphthoic acid salt, a salicylic acid salt, a stearic acid salt, a muconic acid salt, a butyric acid salt, a phenylacetic acid salt, a phenylbutyric acid salt, a valproic acid salt, and the like; (2) salts formed when an acidic proton present in the parent compound is replaced by a metal ion, e.g., an alkali metal ion (e.g. a lithium salt, a sodium salt, or a potassium salt), an alkaline earth ion (e.g. a magnesium salt, or a calcium salt), or an aluminum ion (e.g. an aluminum salt). In some cases, compounds described herein may coordinate with an organic base to form a salt, such as, but not limited to, an ethanolamine salt, a diethanolamine salt, a triethanolamine salt, a tromethamine salt, a N-methylglucamine salt, a dicyclohexylamine salt, or a tris(hydroxymethyl)methylamine salt. In other cases, compounds described herein may form salts with amino acids such as, but not limited to, an arginine salt, a lysine salt, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

It should be understood that a reference to a pharmaceutically acceptable salt includes the solvent addition forms. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ-specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation. In yet other embodiments, the compound described herein is administered topically.

In some embodiments described herein, a therapeutically effective amount of a compound of Formula I, Formula II, Formula Ila, or Formula III, or a pharmaceutically acceptable salt or solvate thereof, is administered by intravenous delivery to a subject in need thereof. In some embodiments described herein, a therapeutically effective amount of a compound of Formula I, Formula II, Formula IIa, or Formula III, or a pharmaceutically acceptable salt or solvate thereof, is administered by intravenous delivery to a subject in need thereof, wherein the intravenous delivery is selected from bolus injection, intravenous drip, and infusion pump. In some embodiments described herein, a therapeutically effective amount of a compound of Formula I, or a pharmaceutically acceptable salt or solvate thereof, is administered by intravenous delivery to a subject in need thereof. In some embodiments described herein, a therapeutically effective amount of a compound of Formula I, or a pharmaceutically acceptable salt or solvate thereof, is administered by intravenous delivery to a subject in need thereof, wherein the intravenous delivery is selected from bolus injection, intravenous drip, and infusion pump. In some embodiments described herein, a therapeutically effective amount of a compound of Formula II, or a pharmaceutically acceptable salt or solvate thereof, is administered by intravenous delivery to a subject in need thereof. In some embodiments described herein, a therapeutically effective amount of a compound of Formula II, or a pharmaceutically acceptable salt or solvate thereof, is administered by intravenous delivery to a subject in need thereof, wherein the intravenous delivery is selected from bolus injection, intravenous drip, and infusion pump. In some embodiments described herein, a therapeutically effective amount of a compound of Formula IIa, or a pharmaceutically acceptable salt or solvate thereof, is administered by intravenous delivery to a subject in need thereof. In some embodiments described herein, a therapeutically effective amount of a compound of Formula IIa, or a pharmaceutically acceptable salt or solvate thereof, is administered by intravenous delivery to a subject in need thereof, wherein the intravenous delivery is selected from bolus injection, intravenous drip, and infusion pump. In some embodiments described herein, a therapeutically effective amount of a compound of Formula III, or a pharmaceutically acceptable salt or solvate thereof, is administered by intravenous delivery to a subject in need thereof. In some embodiments described herein, a therapeutically effective amount of a compound of Formula III, or a pharmaceutically acceptable salt or solvate thereof, is administered by intravenous delivery to a subject in need thereof, wherein the intravenous delivery is selected from bolus injection, intravenous drip, and infusion pump.

### Pharmaceutical Compositions/Formulations

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999), herein incorporated by reference for such disclosure.

Provided herein are pharmaceutical compositions that include a compound of Formula I, Formula II, Formula IIa, or Formula III, or a pharmaceutically acceptable salt thereof, and at least one pharmaceutically acceptable inactive ingredient. In some embodiments is a pharmaceutical composition that includes a compound of Formula I, or a pharmaceutically acceptable salt thereof, and at least one pharmaceutically acceptable excipient. In some embodiments is a pharmaceutical composition that includes a compound of Formula II, or a pharmaceutically acceptable salt thereof, and at least one pharmaceutically acceptable excipient. In some embodiments is a pharmaceutical composition that includes a compound of Formula IIa, or a pharmaceutically acceptable salt thereof, and at least one pharmaceutically acceptable excipient. In some embodiments is a pharmaceutical composition that includes a compound of Formula III, or a pharmaceutically acceptable salt thereof, and at least one pharmaceutically acceptable excipient. In some embodiments, the compounds described herein are administered as pharmaceutical compositions in which a compound of Formula I, Formula II, Formula IIa, or Formula III, or a pharmaceutically acceptable salt thereof, is mixed with other active ingredients, as in combination therapy. In other embodiments, the pharmaceutical compositions include other medicinal or pharmaceutical agents, carriers, adjuvants, preserving, stabilizing, wetting or emulsifying agents, solution promoters, salts for regulating the osmotic pressure, and/or buffers. In yet other embodiments, the pharmaceutical compositions include other therapeutically valuable substances.

In some embodiments, the compounds described herein can be used as an adjuvant for a systemic treatment with N-acetylcysteine, corticosteroids, and vasopressors. In some embodiments, the compounds described herein can be used as an adjuvant for systemic treatment with N-acetylcysteine. In some embodiments, the compounds described herein can be used as an adjuvant for systemic treatment with corticosteroids. In some embodiments, the compounds described herein can be used as an adjuvant for systemic treatment with vasopressors, including but not limited to vasopressin and catecholamines.

A pharmaceutical composition, as used herein, refers to a mixture of a compound of Formula I, Formula II, Formula IIa, or Formula III, or a pharmaceutically acceptable salt thereof, with other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to a mammal.

A therapeutically effective amount can vary widely depending on the severity of the disease, the age and relative health of the subject, the potency of the compound used and other factors. The compounds can be used singly or in combination with one or more therapeutic agents as components of mixtures.

The pharmaceutical formulations described herein are administered to a subject by appropriate administration routes, including but not limited to, oral, parenteral (e.g., intravenous, intravitreal, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a compound of Formula I, Formula II, Formula IIa, or Formula III, or a pharmaceutically acceptable salt thereof, are manufactured in a conventional manner, such as, by way of example only, by means of conventional mixing, dissolving, granulating, dragee-making, levigating, emulsifying, encapsulating, entrapping or compression processes.

The pharmaceutical compositions will include at least one compound of Formula I, Formula II, Formula IIa, or Formula III, as an active ingredient in free-acid or free-base form, or in a pharmaceutically acceptable salt form. In addition, the methods and pharmaceutical compositions described herein include the use of N-oxides (if appropriate), crystalline forms, amorphous phases, as well as active metabolites of these compounds having the same type of activity. In some embodiments, compounds described herein exist in unsolvated form or in solvated forms with pharmaceutically acceptable solvents such as water, ethanol, and the like. The solvated forms of the compounds presented herein are also considered to be disclosed herein.

In some embodiments, solid oral dosage forms are prepared by mixing a compound of Formula I, Formula II, Formula IIa, or Formula III, or a pharmaceutically acceptable salt thereof, with one or more of the following: antioxidants, flavoring agents, and carrier materials such as binders, suspending agents, disintegration agents, filling agents, surfactants, solubilizers, stabilizers, lubricants, wetting agents, and diluents.

In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder, a capsule, solid dispersion, solid solution, bioerodible dosage form, controlled release formulations, pulsatile release dosage forms, multiparticulate dosage forms, beads, pellets, granules. In other embodiments, the pharmaceutical formulation is in the form of a powder. In still other embodiments, the pharmaceutical formulation is in the form of a tablet. In other embodiments, pharmaceutical formulation is in the form of a capsule.

In some embodiments, solid dosage forms, e.g., tablets, effervescent tablets, and capsules, are prepared by mixing particles of a compound of Formula I, Formula II, Formula IIa, or Formula III, or a pharmaceutically acceptable salt thereof, with one or more pharmaceutical excipients to form a bulk blend composition. The bulk blend is readily subdivided into equally effective unit dosage forms, such as tablets, pills, and capsules. In some embodiments, the individual unit dosages include film coatings. These formulations are manufactured by conventional formulation techniques.

Conventional formulation techniques include, e.g., one or a combination of methods: (1) dry mixing, (2) direct compression, (3) milling, (4) dry or non-aqueous granulation, (5) wet granulation, or (6) fusion. Other methods include, e.g., spray drying, pan coating, melt granulation, granulation, fluidized bed spray drying or coating (e.g., wurster coating), tangential coating, top spraying, tableting, extruding and the like.

In some embodiments, a compound of Formula I, Formula II, or Formula III, or a pharmaceutically acceptable salt thereof, is formulated into a pharmaceutical composition suitable for intravitreal, intramuscular, subcutaneous, or intravenous injection. In one aspect, formulations suitable for intravitreal, intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. In some embodiments, formulations suitable for subcutaneous injection contain additives such as preserving, wetting, emulsifying, and dispensing agents.

In some embodiments, a compound of Formula I, Formula II, or Formula III, or a pharmaceutically acceptable salt thereof, is formulated into a pharmaceutical composition suitable for intravenous injection. In some embodiments, a compound of Formula II or Formula IIa, or a pharmaceutically acceptable salt thereof, is formulated into a pharmaceutical composition suitable for intravenous injection. Examples of intravenous injections include but are not limited to bolus injection, intravenous drip, and infusion pump.

For transmucosal administration, penetrants appropriate to the barrier to be permeated are used in the formulation. Such penetrants are generally known in the art.

In certain embodiments, delivery systems for pharmaceutical compounds may be employed, such as, for example, nanoparticles, implants, mucoadhesives, liposomes and emulsions. In certain embodiments, compositions provided herein can also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, the compounds described herein may be administered topically and can be formulated into a variety of topically administrable compositions, such as solutions, suspensions, sprays, lotions, gels, pastes, medicated sticks, foams, balms, creams or ointments.

Such pharmaceutical compounds can contain solubilizers, stabilizers, tonicity enhancing agents, buffers and preservatives.

### Combination Treatments

In certain instances, it is appropriate to administer at least one compound of Formula I, Formula II, Formula IIa, or Formula III, or a pharmaceutically acceptable salt thereof, in combination with one or more other therapeutic agents.

In one embodiment, the therapeutic effectiveness of one of the compounds described herein is enhanced by administration of an adjuvant (i.e., by itself the adjuvant may have minimal therapeutic benefit, but in combination with another therapeutic agent, the overall therapeutic benefit to the patient is enhanced). Or, in some embodiments, the benefit experienced by a patient is increased by administering one of the compounds described herein with another therapeutic agent (which also includes a therapeutic regimen) that also has therapeutic benefit.

In one specific embodiment, a compound of Formula I, Formula II, Formula IIa, or Formula III, or a pharmaceutically acceptable salt thereof, is co-administered with a second therapeutic agent, wherein the compound of Formula I, Formula II, Formula IIa, or Formula III, or a pharmaceutically acceptable salt thereof, and the second therapeutic agent modulate different aspects of the disease, disorder or condition being treated, thereby providing a greater overall benefit than administration of either therapeutic agent alone.

In some embodiments, the second therapeutic agent is an anti-diabetic agent. In some embodiments, the second therapeutic agent is an anti-diabetic agent selected from biguanides, sulfonylureas, meglitinide, thiazolidinediones, dipeptidyl peptidase 4 (DPP-4) inhibitors, sodium-glucose cotransporter (SGLT2) inhibitors, and α-glucosidase inhibitors.

In some embodiments, the second therapeutic agent is N-acetylcysteine. In some embodiments, the second therapeutic agent is a corticosteroid. In some embodiments, the second therapeutic agent is a vasopressor. In some embodiments, the vasopressor is vasopressin or catecholamine. In some embodiments, the vasopressor is vasopressin. In some embodiments, the vasopressor is catecholamine.

In any case, regardless of the disease, disorder or condition being treated, the overall benefit experienced by the patient may simply be additive of the two therapeutic agents or the patient may experience a synergistic benefit.

In certain embodiments, different therapeutically-effective dosages of the compounds disclosed herein will be utilized in formulating pharmaceutical composition and/or in treatment regimens when the compounds disclosed herein are administered in combination with one or more additional agent, such as an additional therapeutically effective drug, an adjuvant or the like. Therapeutically-effective dosages of drugs and other agents for use in combination treatment regimens can be determined by means similar to those set forth hereinabove for the actives themselves. Furthermore, the methods of prevention/treatment described herein encompasses the use of metronomic dosing, i.e., providing more frequent, lower doses in order to minimize toxic side effects. In some embodiments, a combination treatment regimen encompasses treatment regimens in which administration of a compound of Formula I, Formula II, Formula IIa, or Formula III, or a pharmaceutically acceptable salt thereof, is initiated prior to, during, or after treatment with a second agent, and continues until any time during treatment with the second agent or after termination of treatment with the second agent. It also includes treatments in which a compound of Formula I, Formula II, Formula IIa, or Formula III, or a pharmaceutically acceptable salt thereof, and the second agent being used in combination are administered simultaneously or at different times and/or at decreasing or increasing intervals during the treatment period. Combination treatment further includes periodic treatments that start and stop at various times to assist with the clinical management of the patient.

It is understood that the dosage regimen to treat, prevent, or ameliorate the condition(s) for which relief is sought, is modified in accordance with a variety of factors (e.g. the disease, disorder or condition from which the subject suffers; the age, weight, sex, diet, and medical condition of the subject). Thus, in some instances, the dosage regimen actually employed varies and, in some embodiments, deviates from the dosage regimens set forth herein.

For combination therapies described herein, dosages of the co-administered compounds vary depending on the type of co-drug employed, on the specific drug employed, on the disease or condition being treated and so forth. In additional embodiments, when co-administered with one or more other therapeutic agents, the compound provided herein is administered either simultaneously with the one or more other therapeutic agents, or sequentially.

In combination therapies, the multiple therapeutic agents (one of which is one of the compounds described herein) are administered in any order or even simultaneously. If administration is simultaneous, the multiple therapeutic agents are, by way of example only, provided in a single, unified form, or in multiple forms (e.g., as a single pill or as two separate pills).

The compounds of Formula I, Formula II, Formula IIa, or Formula III, or a pharmaceutically acceptable salt thereof, as well as combination therapies, are administered before, during or after the occurrence of a disease or condition, and the timing of administering the composition containing a compound varies. Thus, in one embodiment, the compounds described herein are used as a prophylactic and are administered continuously to subjects with a propensity to develop conditions or diseases in order to prevent the occurrence of the disease or condition. In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each subject. For example, in specific embodiments, a compound described herein or a formulation containing the compound is administered for at least 2 weeks, about 1 month to about 5 years.

### EXAMPLES

These examples are provided for illustrative purposes only and not to limit the scope of the claims provided herein.

### Example 1: Organ-Distribution of ASK1 Inhibitor in Rats after Intravenous Administration

The objective of this study was to investigate organ (liver, heart, kidney, pancreas, spleen and skin) distribution of ASK1 inhibitor in male Sprague Dawley rats following a single intravenous (IV) administration at a dose of 10 mg/kg. Three rats are administered intravenously with a solution formulation of ASK1 inhibitor 2-(6-(4-cyclopropyl-4H-1,2,4-triazol-3-yl)pyridin-2-yl)-5-(morpholine-4-carbonyl)isoindolin-1-one in 5% NMP, 15% Solutol HS-15, 20% PEG-400 and 60% HpBetaCD at 10 mg/kg dose. 2-(6-(4-Cyclopropyl-4H-1,2,4-triazol-3-yl)pyridin-2-yl)-5-(morpholine-4-carbonyl)isoindolin-1-one was prepared as described in US2018/0291002 which is herein incorporated by reference in its entirety.

Blood samples (approximately 120 μL) were collected under light isoflurane anesthesia from retro orbital plexus from a set of three rats at 1 h post IV injection. Immediately after blood collection, plasma was harvested by centrifugation at 4000 rpm, 10 min at 40° C. and samples were stored at −70±10° C. until bioanalysis. Animals were sacrificed immediately followed by abdominal vena-cava cut open and whole body was perfused from heart using 10 mL of normal saline. After isolation, tissue samples were rinsed three times in ice cold normal saline (for 5-10 seconds/rinsed using ˜5-10 mL normal saline in disposable petri dish for each rinse, dried on blotting paper and weighed. Tissue (liver, heart, kidney, pancreas, spleen and skin) samples were homogenized using ice-cold phosphate buffer saline (pH−7.4). Total homogenate volume was five times the tissue weight. All homogenates were stored below −70±10° C. until bioanalysis.

Plasma, liver, heart, kidney, pancreas, spleen, and skin samples were quantified by fit-for-purpose LC-MS/MS method.

Following a single intravenous administration of ASK1 inhibitor 2-(6-(4-cyclopropyl-4H-1,2,4-triazol-3-yl)pyridin-2-yl)-5-(morpholine-4-carbonyl)isoindolin-1-one to male Sprague Dawley rats at 10 mg/kg dose, plasma and tissue concentrations are quantifiable at 1 hr. Tissue-to-plasma ratio at 1 hr were: Liver: 9.37; Heart: 1.11; Kidney: 5.99; Pancreas: 0.99; Spleen: 0.89; Skin: 0.46.

### Example 2: Organ-Distribution of ASK1 Inhibitor in Rats after Oral Administration

The objective of this study was to investigate organ (liver, heart, kidney) distribution of ASK1 inhibitor in male Sprague Dawley rats following a single oral (PO) administration at a dose of 300 mg/kg. Three rats are administered orally with suspension formulation of ASK1 inhibitor 2-(6-(4-cyclopropyl-4H-1,2,4-triazol-3-yl)pyridin-2-yl)-5-(morpholine-4-carbonyl)isoindolin-1-one in a formulation of 0.1% Tween 80 and 99.9% HPMC (0.5% w/v in water) at 300 mg/kg dose.

Blood samples (approximately 120 μL) were collected under light isoflurane anesthesia from retro orbital plexus from a set of three rats at 1 h post PO administration. Immediately after blood collection, plasma was harvested by centrifugation at 4000 rpm, 10 min at 40° C. and samples were stored at −70±10° C. until bioanalysis. Animals were sacrificed immediately followed by abdominal vena-cava cut open and whole body was perfused from heart using 10 mL of normal saline. After isolation, tissue samples were rinsed three times in ice cold normal saline (for 5-10 seconds/rinsed using ˜5-10 mL normal saline in disposable petri dish for each rinse, dried on blotting paper and weighed. Tissue (liver, heart, kidney) samples were homogenized using ice-cold phosphate buffer saline (pH−7.4). Total homogenate volume was five times the tissue weight. All homogenates were stored below −70±10° C. until bioanalysis.

Plasma, liver, heart, kidney, pancreas, spleen, and skin samples were quantified by fit-for-purpose LC-MS/MS method.

Following a single oral administration of ASK1 inhibitor 2-(6-(4-cyclopropyl-4H-1,2,4-triazol-3-yl)pyridin-2-yl)-5-(morpholine-4-carbonyl)isoindolin-1-one to male Sprague Dawley rats at 300 mg/kg dose, plasma and tissue concentrations are quantifiable at 1 hr. Tissue-to-plasma ratio at 1 hr were: Liver: 13.77; Heart: 1.13; Kidney: 7.47.

### Example 3: Efficacy of Intravenous Treatment with ASK1 Inhibitor in Liver Injury Due to Acute Alcoholic Hepatitis (AH)

The goal of the study is to evaluate whether IV administered ASK1 inhibitor can reduce liver injury due to acute alcoholic hepatitis (AH).

Seven-day acclimated C57Bl/6 male mice were treated with pyrazole for 2-days to induce CYP2E, prior to ASK1 inhibitor and lipopolysaccharide (LPS) administration. On day 3, ASK1 inhibitor 2-(6-(4-cyclopropyl-4H-1,2,4-triazol-3-yl)pyridin-2-yl)-5-(morpholine-4-carbonyl)isoindolin-1-one was administered by IV injection via tail vein at 0.3 and 3 mg/kg or vehicle twice (3 hours apart). LPS was administered intraperitoneally 30 min after the first dose of 2-(6-(4-cyclopropyl-4H-1,2,4-triazol-3-yl)pyridin-2-yl)-5-(morpholine-4-carbonyl)isoindolin-1-one. LPS administration emulates the inflammation by gut bacteria penetrating the intestinal wall following binge drinking. 24 h after LPS administration, plasma was collected for ALT analysis to estimate reduction of liver injury following intravenous treatment with ASK1 inhibitor.

Intravenous treatment of ASK1 inhibitor 2-(6-(4-cyclopropyl-4H-1,2,4-triazol-3-yl)pyridin-2-yl)-5-(morpholine-4-carbonyl)isoindolin-1-one significantly reduced liver injury as reflected by 74-85% lowered plasma ALT levels.

### Example 4: Efficacy of Intravenous Treatment with ASK1 Inhibitor in Fulminant Hepatitis Due Acetaminophen Overdose Model

The goal of the study is to evaluate whether IV administered ASK1 inhibitors described herein can reduce liver injury due to acetaminophen (APAP) overdose plasma ALT and reduce liver biomarkers of ASK1 signaling in a mouse model.

Seven-day acclimated C57Bl/6 male mice are IV injected via tail vein or the like with ASK1 inhibitors described herein at the dose range of 0.1-10 mg/kg or vehicle 30 min prior to APAP injection (300 mg/kg ip). At 6 or 24 h, animals are anesthetized with CO2 or isoflurane and blood drawn via cardiac puncture into heparinized syringes for subsequent determination of alanine aminotransferase (ALT) activity. Phospho-JNK, phospho-p38, total JNK and p38 and beta-actin are quantified by western blot using liver samples.

### Example 5: Phase II Study—Evaluation of ASK1 Inhibitor in Patients at Risk of Developing Cardiac Surgery Associated-Acute Kidney Injury (CSA-AKI)

The study is designed to include patients scheduled for cardiac surgery who are considered at increased risk for developing CSA-AKI.

Stage 1 consists of 140 patients randomized in a 1:1:1:1 ratio (approximately 35 patients per treatment arm) to Placebo or to one of three (3) ASK1 inhibitor dose arms:
1. pre-surgery Placebo followed by three (3) post-surgery Placebo doses, or
2. pre-surgery low dose of ASK1 inhibitor followed by three (3) post-surgery doses at the low dose, or
3. pre-surgery middle dose of ASK1 inhibitor followed by three (3) post-surgery doses at the low dose, or
4. pre-surgery high dose of ASK1 inhibitor followed by three (3) post-surgery doses at the low dose

Study treatment (ASK1 inhibitor or placebo) will consist of one 60-minute IV infusion administered prior to surgery, followed by a 60-minute IV infusion administered, beginning in the early post-operative period, and followed by two (2) additional 60-minute IV infusions administered, on consecutive days post-cardiac surgery.

The primary endpoint for the evaluation of efficacy in patients receiving ASK1 inhibitor, as compared to patients receiving Placebo, will be the proportion of patients developing CSA-AKI as measured by the Kidney Disease Improving Global Outcomes (KDIGO) criteria, as follows:
Increase in Serum creatinine (SCr) by ≥0.3 mg/dl (>26.5 μmol/l) within 48 hours post-surgery; or
Increase in SCr to ≥1.5 times a baseline which has been measured in the previous 7 days; or
Urine volume ≤0.5 ml/kg/h for 6 hours post-operatively
If at least one of these measures is present by the 7 day assessment, a patient will be considered to have developed CSA-AKI.

### Secondary Outcome Measures:

Incidence of AKI [Time Frame: 7 days]
Severity of AKI [Time Frame: 7 days]
Staging of AKI is defined as the following:
   Stage 1: SCr 1.5-1.9 times baseline OR ≥0.3 mg/dL (≥26.5 mol/L) increase, Urine output <0.5 ml/kg/hr for 6-12 hours
   Stage 2: SCr 2.0-2.9 times baseline, Urine output <0.5 ml/kg/h for ≥12 hours
   Stage 3: SCr 3.0 times baseline OR Increase in SCr to ≥5.0 mg/dL (≥353.6 μmol/L) OR Initiation of renal replacement therapy OR In patients <18 years, decrease in eGFR to <35 ml/min per 1.73 m\hat{ }2, Urine output <0.3 ml/kg/h for ≥24 hours OR Anuria for ≥12 hours.
   No AKI is considered the best outcome, and Stage 3 the worst outcome.
Duration of AKI [Time Frame: 7 days OR up to discharge after surgery

### Inclusion Criteria:

Male or female and >18 years of age.
Scheduled for a non-emergent coronary and/or valve surgery procedure requiring CPB, to include:
   coronary artery bypass graft (CABG) alone;
   aortic valve replacement or repair alone, with or without aortic root repair;
   mitral, tricuspid, or pulmonic valve replacement or repair alone;
   simultaneous replacement of several cardiac valves;
   CABG with aortic, mitral, tricuspid, or pulmonic valve replacement or repair;
   CABG with combined cardiac valve replacement or repair.
Have the following risk factors for CSA-AKI:
   eGFR≥20 and <30 ml/min/1.73 m2 OR
   eGFR≥30 and <60 ml/min/1.73 m2 and ONE of the following additional risk factors (other than age≥75 years) OR
   eGFR≥60 ml/min/1.73 m2 and TWO of the following additional risk factors
Additional Risk Factors:
   Age≥75 years;
   Combined valve & coronary surgery;
   Previous cardiac surgery with sternotomy;
   Documented NYHA Class III or IV within 1 year prior to surgery;
   Left ventricular ejection fraction (LVEF)≤35% by invasive or noninvasive diagnostic cardiac imaging—echocardiography, nuclear imaging, computed tomography, magnetic resonance imaging or angiography performed within 90 days prior to surgery. (If LVEF≤35% by any invasive or noninvasive imaging procedure, patient meets the risk factor.)
   Insulin-requiring diabetes;
   Non-insulin-requiring diabetes and the presence of ≥+2 proteinuria on urinalysis (medical history or dipstick);
   Preoperative anemia (hemoglobin<11 g/dl for men and women).

### Exclusion Criteria:

Weight>174 kg or 383 lbs;
The presence of AKI (KDIGO criteria) at the time of screening;
Surgery to be performed without CPB;
Surgery to be performed under conditions of circulatory arrest or hypothermia with rectal temperature<28° Celsius (82.4° Fahrenheit);
eGFR (MDRD)<20 ml/min/1.73 m2;
Surgery for aortic dissection;
Surgery to correct a major congenital heart defect (e.g., Tetralogy of Fallot, transposition of the great vessels, single ventricle, Ebsteins anomaly. Bicuspid aortic valve is not to be considered a congenital heart defect.);
Prior organ transplantation;
Dialysis-dependence;
Administration of iodinated contrast media within 24 hours prior to cardiac surgery;
If received contrast media prior to 24 hours and have AKI as defined by KDIGO criteria;
Cardiogenic shock or haemodynamic instability within the 24 hours prior to surgery, including the anesthesia induction period; as defined by a systolic BP<80 mm Hg and pulse>120 beats per minute (bpm) and requirement for inotropes or vasopressors or other mechanical devices such as intra-aortic balloon counter-pulsation (IABP);
Requirement for any of the following within seven (7) days prior to cardiac surgery:
   defibrillator or permanent pacemaker,
   mechanical ventilation,
   intra-aortic balloon counter-pulsation (IABP),
   left ventricular assist device (LVAD),
   other forms of mechanical circulatory support (MCS);
Cardiopulmonary resuscitation within 14 days prior to cardiac surgery;
Known history of cancer within the past 5 years, except for carcinoma in situ of the cervix or adequately treated basal cell carcinoma of the skin;
Known or suspected sepsis at time of screening;
Known or suspected glomerulonephritis or interstitial nephritis at time of screening;
Confirmed or treated endocarditis within previous 30 days prior to cardiac surgery;
Other current active infection requiring antibiotic treatment;
Inadequate hepatic function, defined as total bilirubin or alanine aminotransferase (ALT) or aspartate aminotransferase (AST)>2 times the upper limit of normal (ULN) at time of screening or Child Pugh Class C liver disease (see appendix 6) or higher;
Any congenital coagulation disorder;
Pregnancy or lactation;
If patient has “Do Not Resuscitate” (DNR) status;
Known hypersensitivity to the study drug or any of its excipients;
Treatment with an investigational drug or participation in an interventional trial within 60 days prior to 1st dose of study drug;
In the opinion of the investigator any disease processes or confounding variables that would inappropriately alter the outcome of the study;
Inability to comply with the requirements of the study protocol.

The examples and embodiments described herein are for illustrative purposes only and various modifications or changes suggested to persons skilled in the art to be included within the spirit and purview of this application and scope of the appended claims.

What is claimed is:

1. A method for treating an organ disease or disorder in a subject in need thereof comprising administering to the subject a therapeutically effective amount of a compound of Formula IIa, or a pharmaceutically acceptable salt or solvate thereof: 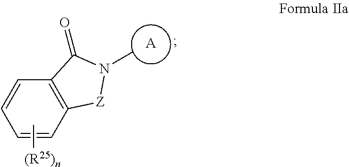
wherein 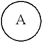
is 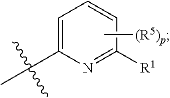
R¹ is 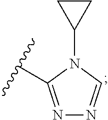 [SMILES: *c1nncn1C1CC1]
Z is C(R⁹)₂;
R⁵ is selected from a group consisting of halogen and C₁₋₆alkyl;
each R²⁵ is independently selected from a group consisting of halogen, —OR⁶, —N(R⁶)₂, —C(═O)OR⁶, —C(═O)N(R⁶)₂, —NR⁶C(═O)N(R⁶)₂, and C₁₋₉heteroaryl selected from pyridinyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl; wherein the C₁₋₉heteroaryl is optionally substituted with one, two, or three substituents selected from the group consisting of halo, C₁₋₆alkyl, C₁₋₆haloalkyl, and C₃₋₈cycloalkyl;
each R⁶ is independently selected from the group consisting of hydrogen, C₁-C₆alkyl, —C₁-C₆alkyl-O—C₁-C₆alkyl, —C₁-C₆alkyl-pyrazole, and C₃-C₈cycloalkyl; or two R⁶ on the same heteroatom are taken together with that heteroatom to which they are attached to form a C₂₋₉heterocycle or a C₂₋₉heteroaryl wherein the heteroaryl is_selected from imidazolyl, pyrazolyl, and pyrrolyl, wherein the C₂₋₉heterocycle or C₂₋₉heteroaryl are optionally substituted with one, two, or three substituents selected from the group consisting of halo, —OR⁸, —SR⁸, —N(R⁸)₂, —C₁₋₆alkyl, —C(═O)R¹⁴, —C(═O)OR¹³, and —N(R¹³)C(═O)R¹⁴;
each R⁸ is independently selected from the group consisting of hydrogen and C₁-C₆alkyl;
each R⁹ is hydrogen;
each R¹³ is independently selected from the group consisting of hydrogen and C₁-C₆alkyl;
each R¹⁴ is independently selected from the group consisting of C₁-C₆alkyl;
n is 0, 1, or 2; and
p is 0 or 1.

2. The method of claim 1, or a pharmaceutically acceptable salt or solvate thereof, wherein n is 1.

3. The method of claim 2, or a pharmaceutically acceptable salt or solvate thereof, wherein
R²⁵ is —C(═O)N(R⁶)₂ and each R⁶ is independently selected from the group consisting of hydrogen, C₁-C₆alkyl, —C₁-C₆alkyl-O—C₁-C₆alkyl, —C₁-C₆alkyl-pyrazole, and C₃-C₈cycloalkyl.

4. The method of claim 2, or a pharmaceutically acceptable salt or solvate thereof, wherein
R²⁵ is —C(═O)N(R⁶)₂ and two R⁶ on the same heteroatom are taken together with that heteroatom to which they are attached to form a C₂₋₉heterocycle or a C₂₋₉heteroaryl wherein the heteroaryl is selected from imidazolyl, pyrazolyl, and pyrrolyl, wherein the C₂₋₉heterocycle or C₂₋₉heteroaryl are optionally substituted with one substituent selected from the group consisting of —OR⁸, —SR⁸, —N(R⁸)₂, —C₁₋₆alkyl, —C(═O)R¹⁴, —C(═O)OR¹³, and —N(R¹³)C(═O)R¹⁴.

5. The method of claim 4, or a pharmaceutically acceptable salt or solvate thereof, wherein R²⁵ is

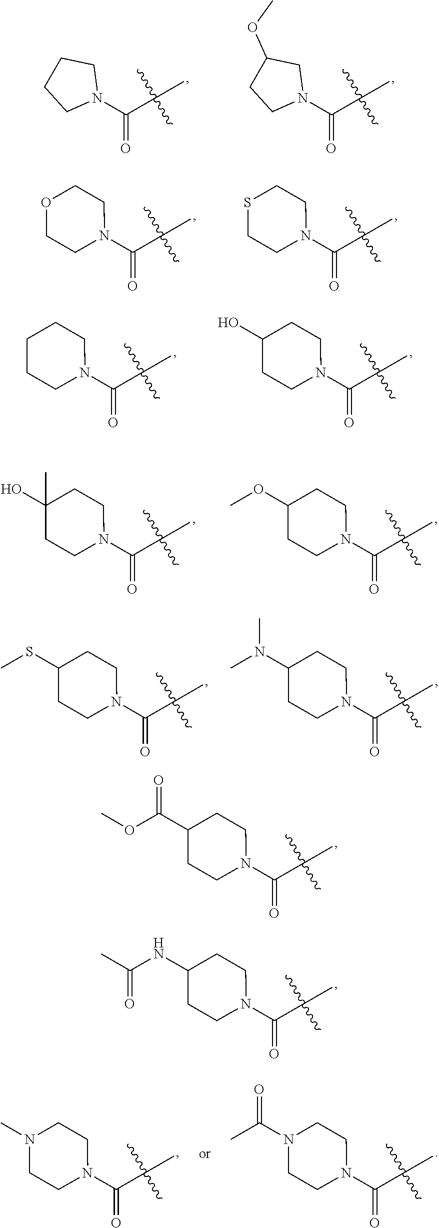

6. The method of claim 5, or a pharmaceutically acceptable salt or solvate thereof, wherein
R²⁵ is

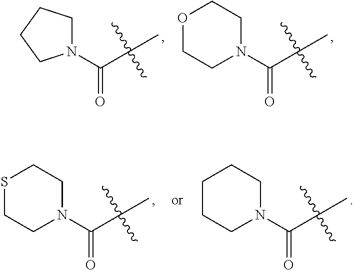 [SMILES: *C(=O)N1CCCC1 ; *C(=O)N1CCOCC1 ; *C(=O)N1CCSCC1 ; *C(=O)N1CCCCC1]

7. The method of claim 1, or a pharmaceutically acceptable salt or solvate thereof, wherein the compound is

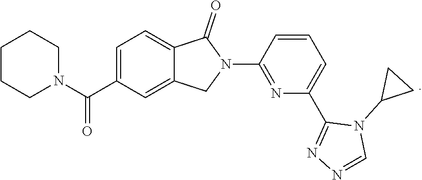 [SMILES: O=C(c1ccc2c(c1)CN(c1cccc(-c3nncn3C3CC3)n1)C2=O)N1CCCCC1]

8. The method of claim 1, or a pharmaceutically acceptable salt or solvate thereof, wherein the compound is

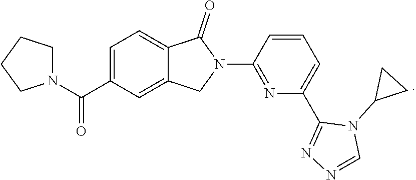 [SMILES: O=C(c1ccc2c(c1)CN(c1cccc(-c3nncn3C3CC3)n1)C2=O)N1CCCC1]

9. The method of claim 1, or a pharmaceutically acceptable salt or solvate thereof, wherein the compound is

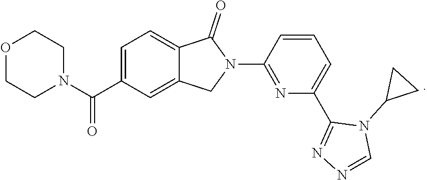 [SMILES: O=C(c1ccc2c(c1)CN(c1cccc(-c3nncn3C3CC3)n1)C2=O)N1CCOCC1]

10. The method of claim 1, or a pharmaceutically acceptable salt or solvate thereof, wherein the compound is

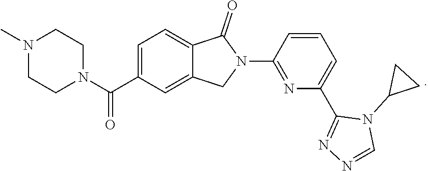 [SMILES: CN1CCN(C(=O)c2ccc3c(c2)CN(c2cccc(-c4nncn4C4CC4)n2)C3=O)CC1]

11. The method of claim 1, or a pharmaceutically acceptable salt or solvate thereof, wherein the compound is

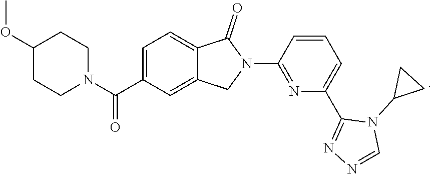 [SMILES: COC1CCN(C(=O)c2ccc3c(c2)CN(c2cccc(-c4nncn4C4CC4)n2)C3=O)CC1]

12. A method for treating an organ disease or disorder in a subject in need thereof comprising administering to the subject a therapeutically effective amount of a compound, or a pharmaceutically acceptable salt or solvate thereof, selected from:

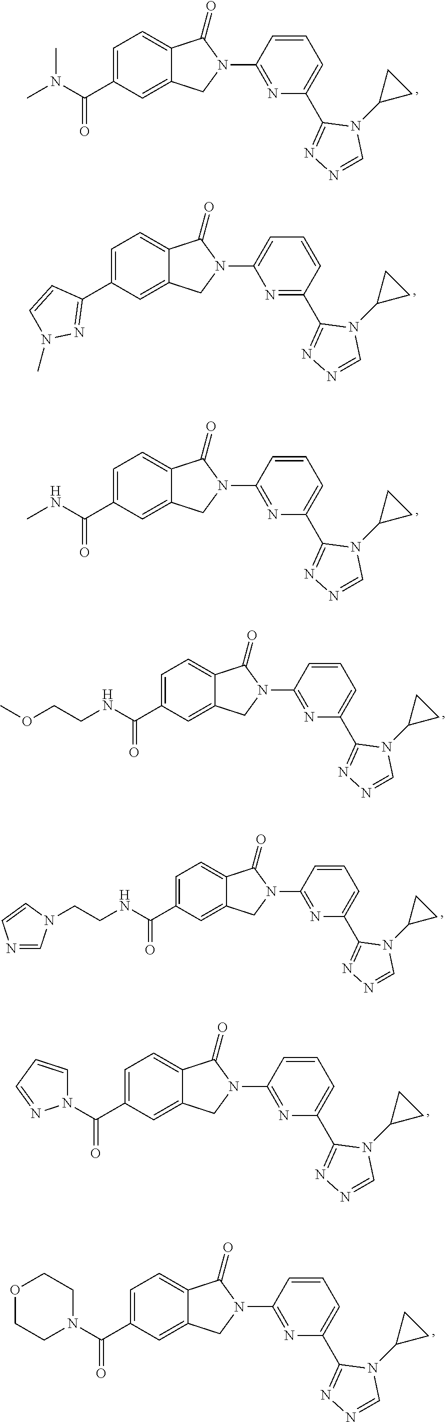

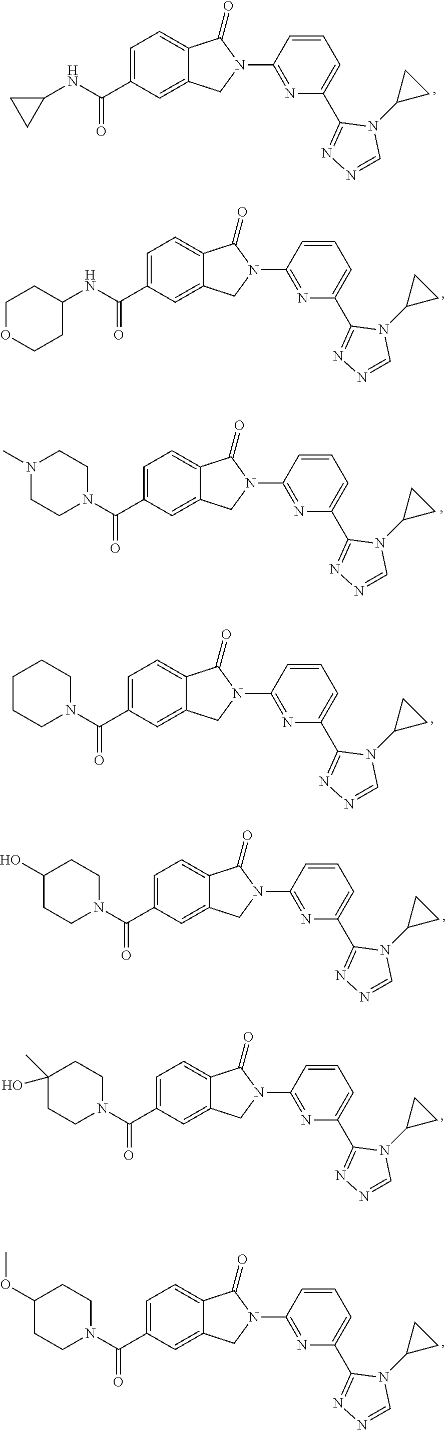

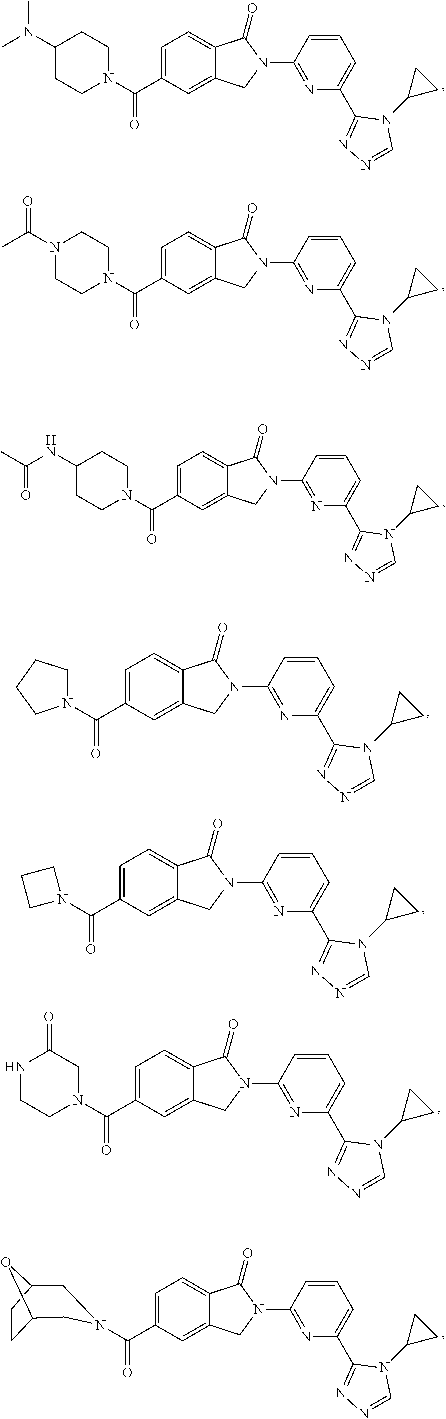

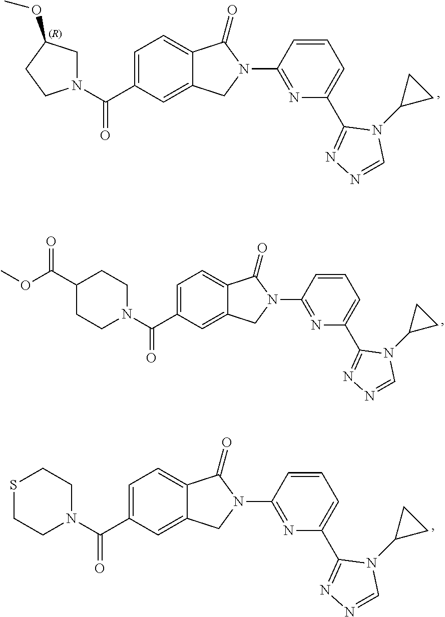

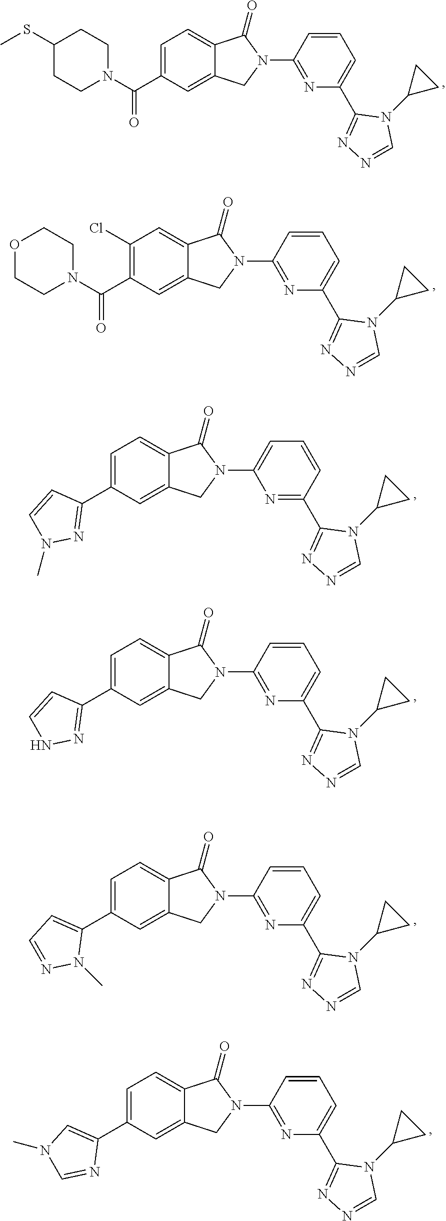 [SMILES: CSC1CCN(C(=O)c2ccc3c(c2)CN(c2cccc(-c4nncn4C4CC4)n2)C3=O)CC1 ; O=C(c1cc2c(cc1Cl)C(=O)N(c1cccc(-c3nncn3C3CC3)n1)C2)N1CCOCC1 ; Cn1ccc(-c2ccc3c(c2)CN(c2cccc(-c4nncn4C4CC4)n2)C3=O)n1 ; O=C1c2ccc(-c3cc[nH]n3)cc2CN1c1cccc(-c2nncn2C2CC2)n1 ; Cn1nccc1-c1ccc2c(c1)CN(c1cccc(-c3nncn3C3CC3)n1)C2=O ; Cn1cnc(-c2ccc3c(c2)CN(c2cccc(-c4nncn4C4CC4)n2)C3=O)c1]

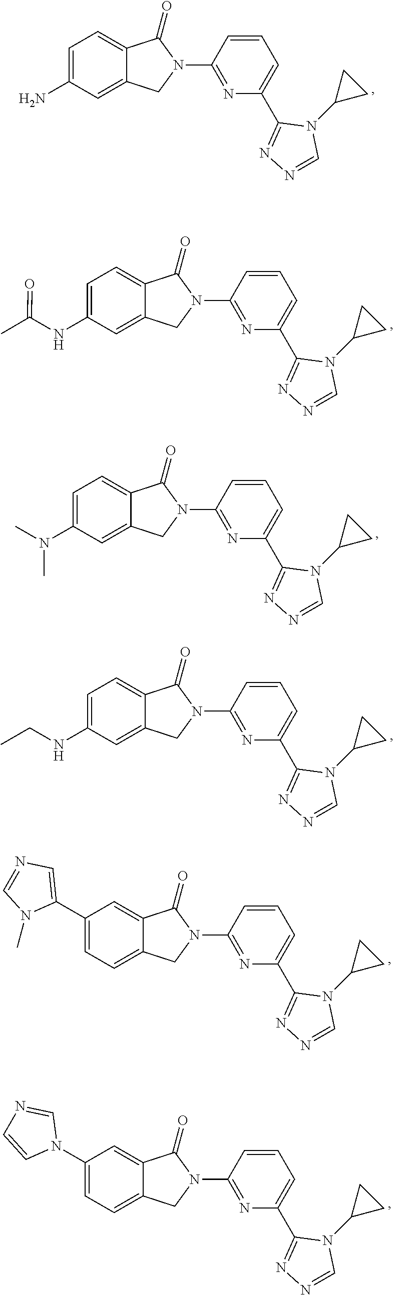 [SMILES: Nc1ccc2c(c1)CN(c1cccc(-c3nncn3C3CC3)n1)C2=O ; CC(=O)Nc1ccc2c(c1)CN(c1cccc(-c3nncn3C3CC3)n1)C2=O ; CN(C)c1ccc2c(c1)CN(c1cccc(-c3nncn3C3CC3)n1)C2=O ; CCNc1ccc2c(c1)CN(c1cccc(-c3nncn3C3CC3)n1)C2=O ; Cn1cncc1-c1ccc2c(c1)C(=O)N(c1cccc(-c3nncn3C3CC3)n1)C2 ; O=C1c2cc(-n3ccnc3)ccc2CN1c1cccc(-c2nncn2C2CC2)n1]

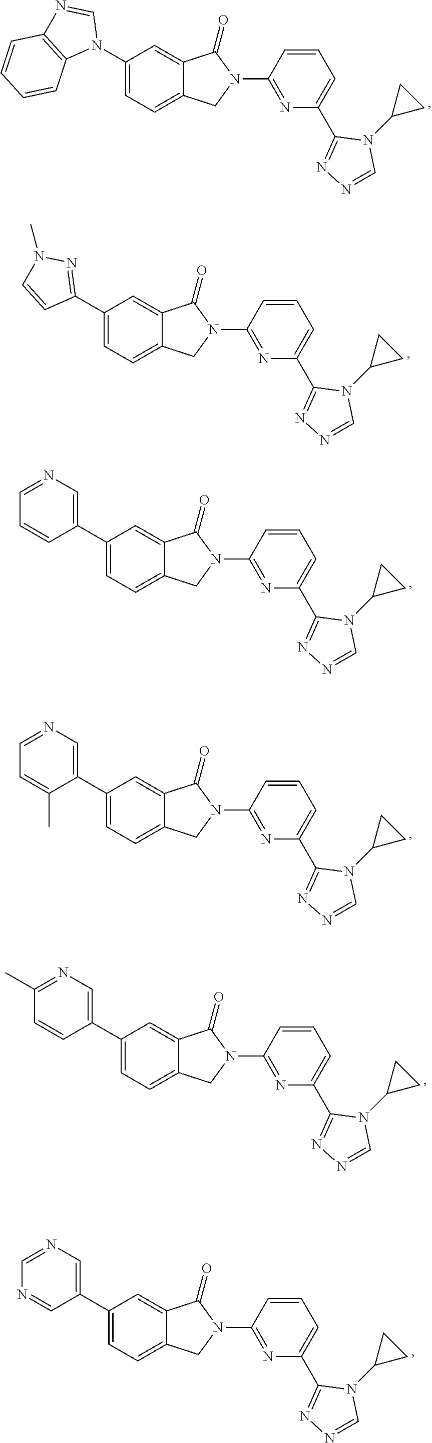 [SMILES: O=C1c2cc(-n3cnc4ccccc43)ccc2CN1c1cccc(-c2nncn2C2CC2)n1 ; Cn1ccc(-c2ccc3c(c2)C(=O)N(c2cccc(-c4nncn4C4CC4)n2)C3)n1 ; O=C1c2cc(-c3cccnc3)ccc2CN1c1cccc(-c2nncn2C2CC2)n1 ; Cc1ccncc1-c1ccc2c(c1)C(=O)N(c1cccc(-c3nncn3C3CC3)n1)C2 ; Cc1ccc(-c2ccc3c(c2)C(=O)N(c2cccc(-c4nncn4C4CC4)n2)C3)cn1 ; O=C1c2cc(-c3cncnc3)ccc2CN1c1cccc(-c2nncn2C2CC2)n1]

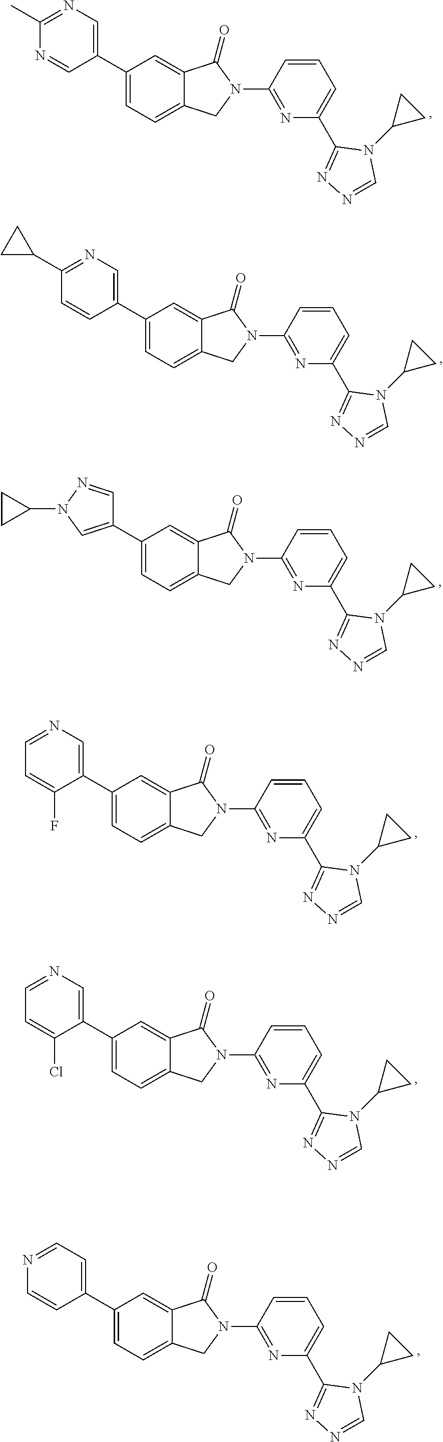 [SMILES: Cc1ncc(-c2ccc3c(c2)C(=O)N(c2cccc(-c4nncn4C4CC4)n2)C3)cn1 ; O=C1c2cc(-c3ccc(C4CC4)nc3)ccc2CN1c1cccc(-c2nncn2C2CC2)n1 ; O=C1c2cc(-c3cnn(C4CC4)c3)ccc2CN1c1cccc(-c2nncn2C2CC2)n1 ; O=C1c2cc(-c3cnccc3F)ccc2CN1c1cccc(-c2nncn2C2CC2)n1 ; O=C1c2cc(-c3cnccc3Cl)ccc2CN1c1cccc(-c2nncn2C2CC2)n1 ; O=C1c2cc(-c3ccncc3)ccc2CN1c1cccc(-c2nncn2C2CC2)n1]

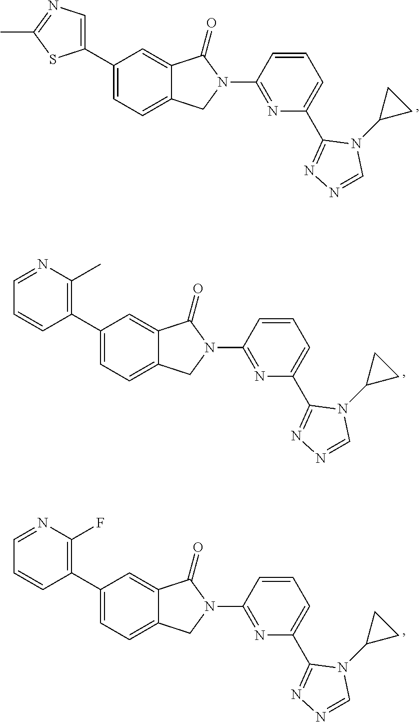 [SMILES: Cc1ncc(-c2ccc3c(c2)C(=O)N(c2cccc(-c4nncn4C4CC4)n2)C3)s1 ; Cc1ncccc1-c1ccc2c(c1)C(=O)N(c1cccc(-c3nncn3C3CC3)n1)C2 ; O=C1c2cc(-c3cccnc3F)ccc2CN1c1cccc(-c2nncn2C2CC2)n1]

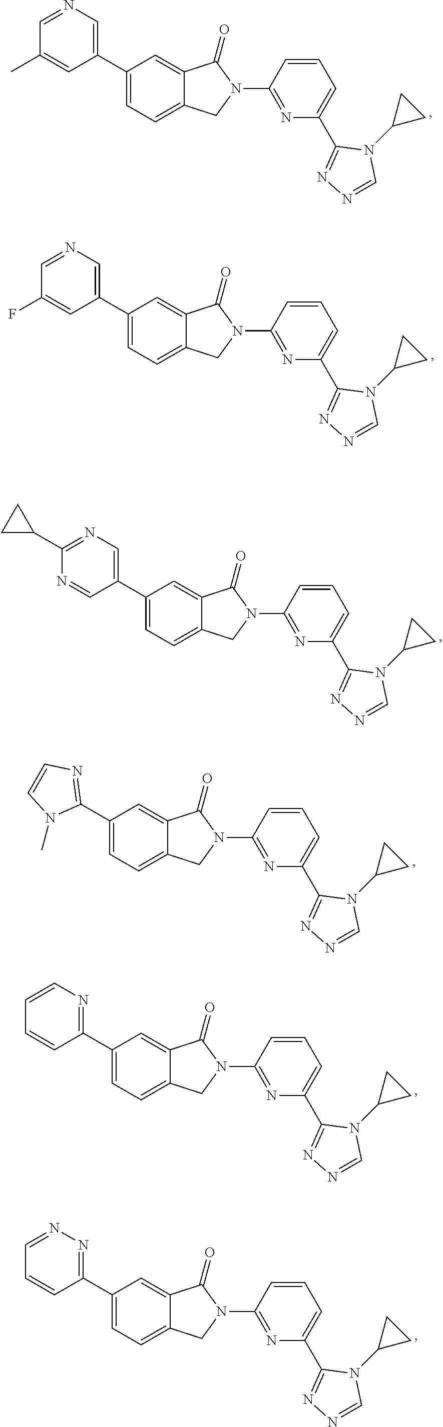 [SMILES: Cc1cncc(-c2ccc3c(c2)C(=O)N(c2cccc(-c4nncn4C4CC4)n2)C3)c1 ; O=C1c2cc(-c3cncc(F)c3)ccc2CN1c1cccc(-c2nncn2C2CC2)n1 ; O=C1c2cc(-c3cnc(C4CC4)nc3)ccc2CN1c1cccc(-c2nncn2C2CC2)n1 ; Cn1ccnc1-c1ccc2c(c1)C(=O)N(c1cccc(-c3nncn3C3CC3)n1)C2 ; O=C1c2cc(-c3ccccn3)ccc2CN1c1cccc(-c2nncn2C2CC2)n1 ; O=C1c2cc(-c3cccnn3)ccc2CN1c1cccc(-c2nncn2C2CC2)n1]

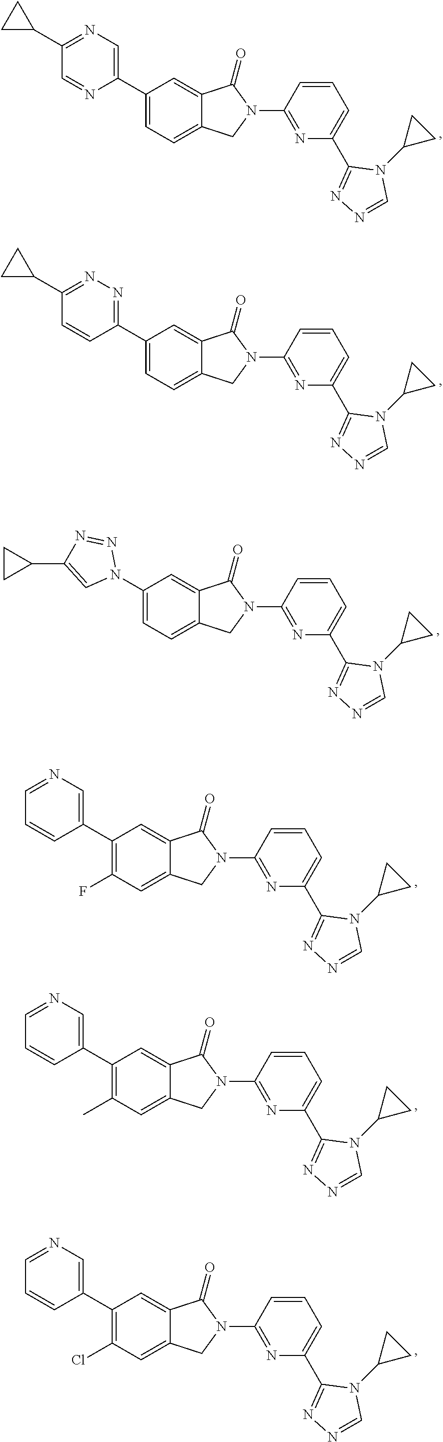 [SMILES: O=C1c2cc(-c3cnc(C4CC4)cn3)ccc2CN1c1cccc(-c2nncn2C2CC2)n1 ; O=C1c2cc(-c3ccc(C4CC4)nn3)ccc2CN1c1cccc(-c2nncn2C2CC2)n1 ; O=C1c2cc(-n3cc(C4CC4)nn3)ccc2CN1c1cccc(-c2nncn2C2CC2)n1 ; O=C1c2cc(-c3cccnc3)c(F)cc2CN1c1cccc(-c2nncn2C2CC2)n1 ; Cc1cc2c(cc1-c1cccnc1)C(=O)N(c1cccc(-c3nncn3C3CC3)n1)C2 ; O=C1c2cc(-c3cccnc3)c(Cl)cc2CN1c1cccc(-c2nncn2C2CC2)n1]

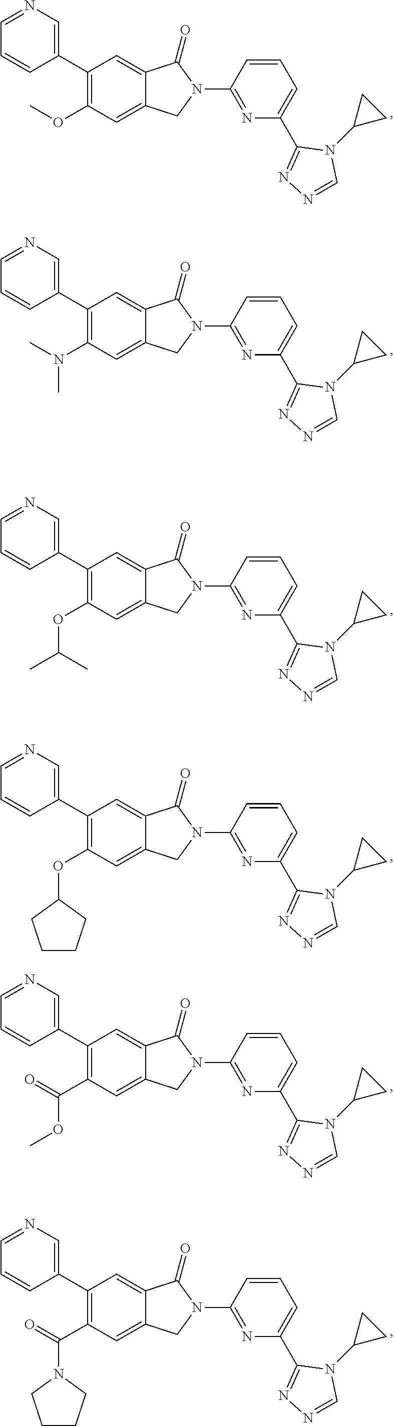

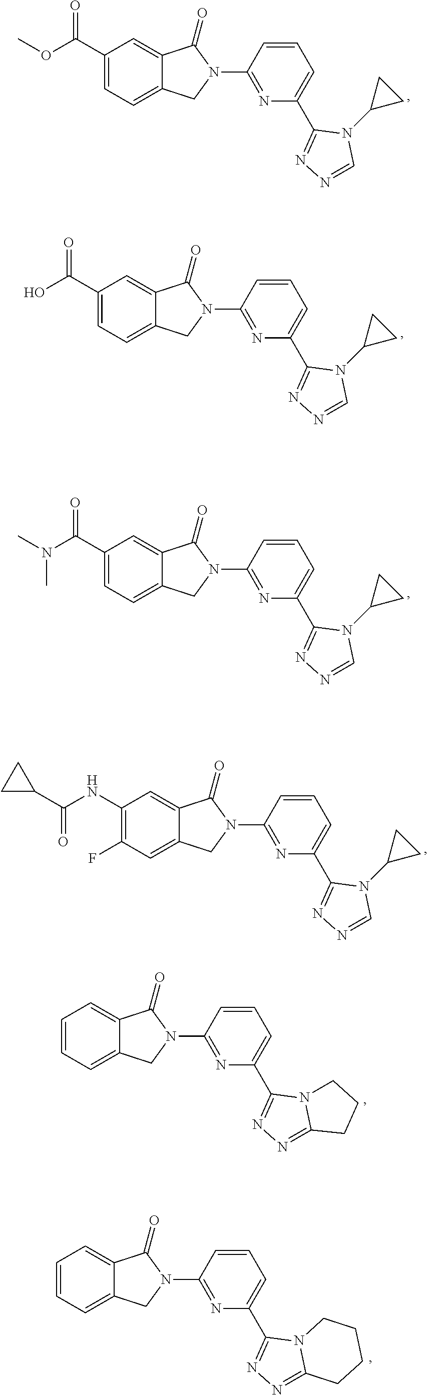 [SMILES: COC(=O)c1ccc2c(c1)C(=O)N(c1cccc(-c3nncn3C3CC3)n1)C2 ; O=C(O)c1ccc2c(c1)C(=O)N(c1cccc(-c3nncn3C3CC3)n1)C2 ; CN(C)C(=O)c1ccc2c(c1)C(=O)N(c1cccc(-c3nncn3C3CC3)n1)C2 ; O=C(Nc1cc2c(cc1F)CN(c1cccc(-c3nncn3C3CC3)n1)C2=O)C1CC1 ; O=C1c2ccccc2CN1c1cccc(-c2nnc3n2CCC3)n1 ; O=C1c2ccccc2CN1c1cccc(-c2nnc3n2CCCC3)n1]

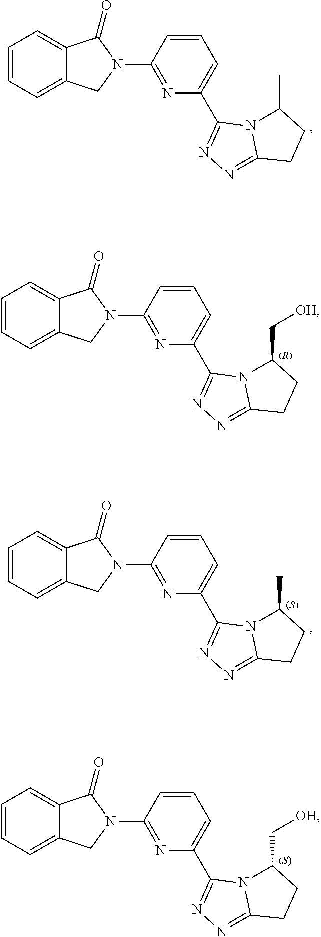

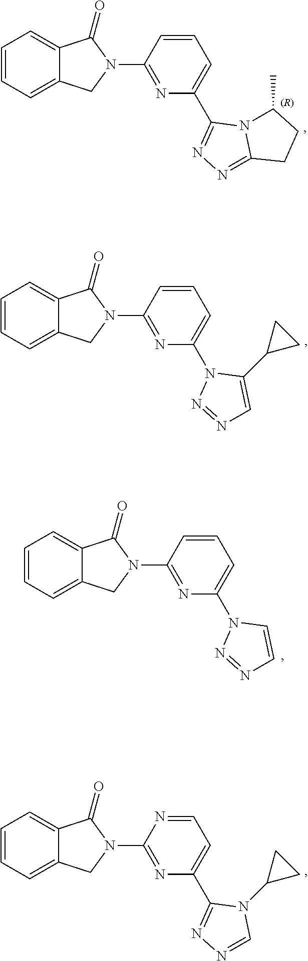

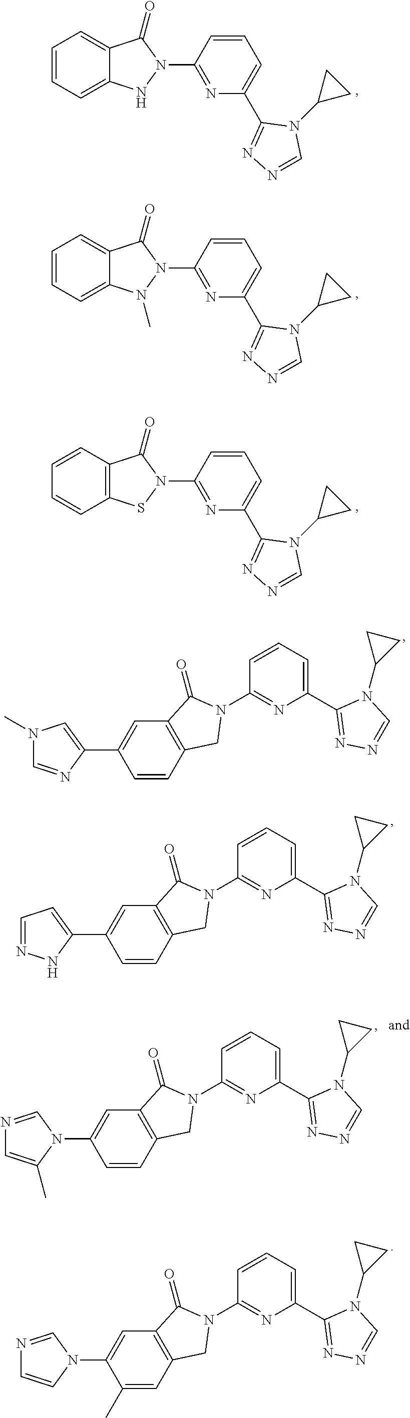

13. The method of any one of claims 1-12, wherein the organ disease or disorder is selected from liver disease or disorder, heart disease or disorder, kidney disease or disorder, pancreas disease or disorder, spleen disease or disorder, and skin disease or disorder.

14. The method of claim 13, wherein the organ disease or disorder is selected from liver disease or disorder, heart disease or disorder, and kidney disease or disorder.

15. The method of claim 14, wherein the organ disease or disorder is a liver disease or disorder.

16. The method of claim 15, wherein the organ disease or disorder is a liver disease or disorder is selected from a liver injury, a drug-induced liver injury, a liver failure, a fulminant or acute liver failure, and an acute-on-chronic liver failure.

17. The method of claim 14, wherein the organ disease or disorder is a heart disease or disorder.

18. The method of claim 17, wherein the organ disease or disorder is a heart disease or disorder selected from ischemia reperfusion injury, ischemia, coronary artery disease, cardiovascular dysfunction from sepsis, drug-induced cardiotoxicity, viral myocarditis, and complications due to heart transplant.

19. The method of claim 14, wherein the organ disease or disorder is a kidney disease or disorder.

20. The method of claim 19, wherein the kidney disease or disorder is an acute kidney injury.

21. The method of claim 20, wherein the kidney disease or disorder is an acute kidney injury due to hypovolemia, hypotension, renal vasoconstriction, glomerular efferent arteriolar vasodilation, prolonged renal ischemia, prolonged renal sepsis, prolonged renal nephrotoxins, kidney transplants, acute tubular necrosis, acute interstitial nephritis, glomerulonephritis, or intratubular obstruction.

22. The method of any one of claims 1-21 wherein the compound is administered by injectable delivery.

23. The method of claim 22, wherein the injectable delivery is an intravenous delivery.

24. The method of claim 23, wherein the intravenous delivery is selected from bolus injection, intravenous drip, and infusion pump.

25. The method of claim 22, wherein the injectable delivery is an intraperitoneal delivery.

26. The method of any one of claims 1-25 further comprising the administration of a second therapeutic agent.

27. The method of claim 26, wherein the second therapeutic agent is selected from N-acetylcysteine, corticosteroids, and vasopressors.
